(19) United States
(12) Patent Application Publication
(10) Pub. No.: US 2026/0008771 A1
(43) Pub. Date: Jan. 8, 2026

(54) PYRIDAZINE BASED SMALL MOLECULE INHIBITOR OF COGNITIVE IMPAIRMENT

(71) Applicants: The Trustees of Columbia University in the City of New York, New York, NY (US); Northwestern University, Chicago, IL (US)

(72) Inventors: Daniel Martin WATTERSON, Douglas, MI (US); Saktimayee Mitra ROY, Evanston, IL (US); Ottavio ARANCIO, New York, NY (US); Erica ACQUARONE, New York, NY (US)

(21) Appl. No.: 19/259,970

(22) Filed: Jul. 3, 2025

Related U.S. Application Data
(63) Continuation-in-part of application No. PCT/US2024/010282, filed on Jan. 4, 2024.
(60) Provisional application No. 63/478,463, filed on Jan. 4, 2023.

(51) Int. Cl. C07D 403/12; A61K 31/501; A61K 31/506; A61P 25/00; A61P 25/28; C07D 401/04; C07D 401/12; C07D 401/14; C07D 403/04; C07D 403/14

(52) U.S. Cl. CPC C07D 403/12; A61K 31/501; A61K 31/506; A61P 25/00; A61P 25/28; C07D 401/04; C07D 401/12; C07D 401/14; C07D 403/04; C07D 403/14

(57) ABSTRACT

Pyridazine based compounds and pharmaceutical compositions that may be used for treating synaptic and behavioral dysfunction such as that associated with Alzheimer's Disease, tauopathies, Alzheimer's Disease related dementia, and other dementia. Compounds disclosed herein can be used for treatment of neuropsychiatric, cognitive or behavioral disorders, especially those associated with neurodegenerative disorders. Also disclosed is a method for treating neuropsychiatric, cognitive or behavioral disorders in a subject suffering from a neurodegenerative disease by administering these compounds and pharmaceutical compositions to a subject in need thereof.

### CROSS REFERENCE TO RELATED APPLICATION

This application is a continuation-in-part of International Application No. PCT/US2024/010282, filed on Jan. 4, 2024, which claims the benefit of and priority to U.S. Provisional Application No. 63/478,463, filed Jan. 4, 2023, the entire contents of each are hereby incorporated by reference in their entireties.

### STATEMENT REGARDING FEDERALLY-SPONSORED RESEARCH OR DEVELOPMENT

This invention was made with government support under Grant AG066722 awarded by National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Neurodegenerative diseases are characterized by progressive cognitive and behavioral dysfunction that can include memory loss and neuropsychiatric symptoms such as depression, agitation, psychosis and apathy. Current treatments for neuropsychiatric symptoms include antidepressants and antipsychotics but are limited in their utility for many patients and by their side effects.

It would be beneficial to develop compounds and pharmaceutical compositions for treatment of neuropsychiatric, cognitive or behavioral disorders, especially those associated with neurodegenerative disorders, while minimizing unwanted side effects and reducing the underlying pathophysiology of synaptic dysfunction that manifests as cognitive dysfunction, anxiety, and/or depression.

### SUMMARY

The present application discloses pyridazine based small molecule compounds that in some cases attenuate synaptic and behavioral dysfunction associated with neurodegenerative diseases. In some aspects, the compounds disclosed herein can be used for treatment of neuropsychiatric, cognitive or behavioral disorders, especially those associated with neurodegenerative disorders, while minimizing unwanted side effects and reducing the underlying pathophysiology of synaptic dysfunction that manifests as cognitive dysfunction, anxiety, and/or depression. In accordance with one aspect, these compounds are selective attenuators of the serotonin 5-Hydroxytryptamine receptor 2B receptor (5-HT2bR) and lack 5-HT2bR agonist activity. These compounds may be used for the treatment of neurodegenerative diseases such as Alzheimer's disease (AD) and Alzheimer's disease related disorders (ADRD) or provide a highly selective inhibitor of 5HT2bR function in precision medicine evaluations. The treatment disclosed herein can ameliorate the breakdown of communication between neurons known as synaptic dysfunction, a key contributor to the dysregulation of cell communication manifest as cognitive or behavioral impairment.

In accordance with one aspect, a compound of Formula (I):

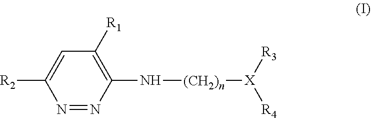

wherein R₁ is CN, C₁-C₆ alkyl, aryl, or heteroaryl; R₂ is phenyl, benzyl, naphthyl, quinolinyl, isoquinolinyl, or indolyl, wherein said phenyl, benzyl, naphthyl, quinolinyl, isoquinolinyl or indolyl is optionally independently substituted with at least one halogen; X is CH or N; n is 1 or 2; and R₃ and R₄ are independently H, C₁-C₆ alkyl, aryl or heteroaryl, provided that R₃ and R₄ are not both H, or R₃, R₄ together with the X to which they are attached form

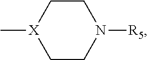

where R₅ is H, C₁-C₆ alkyl, aryl, or heteroaryl; or
a pharmaceutically acceptable salt thereof; or a compound of Formula (II):

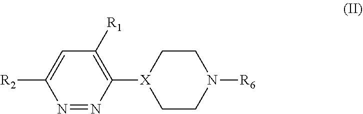

wherein R₁ is CN, C₁-C₆ alkyl, aryl, or heteroaryl; R₂ is benzyl, naphthyl, quinolinyl, isoquinolinyl, or indolyl, wherein said benzyl, naphthyl, quinolinyl, isoquinolinyl or indolyl is optionally independently substituted with at least one halogen; X is CH or N; and R₆ is H, C₁-C₆ alkyl, aryl, or heteroaryl; or
a pharmaceutically acceptable salt thereof; with the proviso that when R₂ is phenyl, R₅ is not H;
or when R₁ and R₂ are both phenyl, neither one of R₃ or R₄ is pyrimidinyl; or when X is C, R₅ is not benzyl is disclosed.

In accordance with one embodiment, a compound of Formula (Ia):

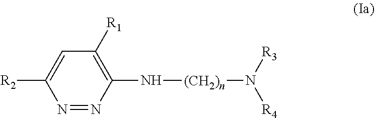

wherein R₁ is CN, C₁-C₆ alkyl, aryl, or heteroaryl; R₂ is phenyl, benzyl, naphthyl, quinolinyl, isoquinolinyl, or indolyl, wherein said phenyl, benzyl, naphthyl, quinolinyl, isoquinolinyl or indolyl is optionally independently substituted with at least one halogen; X is CH or N; n is 1 or 2; and R₃ and R₄ are independently H, C₁-C₆ alkyl, aryl or heteroaryl, provided that R₃ and R₄ are not both H, or R₃, R₄ together with the N to which they are attached form

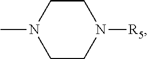

where R₅ is H, C₁-C₆ alkyl, aryl, or heteroaryl or a pharmaceutically acceptable salt thereof; with the proviso that when R₂ is phenyl, R₅ is not H; or when R₁ and R₂ are both phenyl, neither one of R₃ or R₄ is pyrimidinyl; or when X is C, R₅ is not benzyl is disclosed.

In accordance with one embodiment, the disclosure provides a compound of Formula I, wherein R₁ is CN, C₁-C₂ alkyl, phenyl, or pyridyl; n is 2; and R₃, R₄ together with the N to which they are attached form

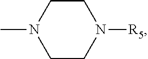

where R₅ is H, C₁-C₂ alkyl, aryl, or heteroaryl containing at least one N in an aryl ring or a pharmaceutically acceptable salt thereof.

In accordance with another embodiment, the disclosure provides a compound of Formula I, wherein R₁ is methyl; R₂ is phenyl, or naphthyl; n is 2; and R₃, R₄ together with the N to which they are attached form

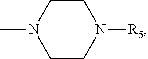

where R₅ is C₁-C₂ alkyl or a pharmaceutically acceptable salt thereof.

In accordance with one embodiment, the disclosure provides a compound of Formula II wherein R₁ is CN, C₁-C₂ alkyl, phenyl, or pyridyl; and R₆ is C₁-C₂ alkyl, or heteroaryl containing at least one N in an aryl ring or a pharmaceutically acceptable salt thereof.

In accordance with another embodiment, the disclosure provides a compound of Formula II, wherein R₁ is C₁-C₂ alkyl, phenyl, or pyridyl; and R₆ is methyl, pyridinyl or pyrimidinyl or a pharmaceutically acceptable salt thereof.

In accordance with some embodiments, the disclosure provides a compound having one of the following structures:

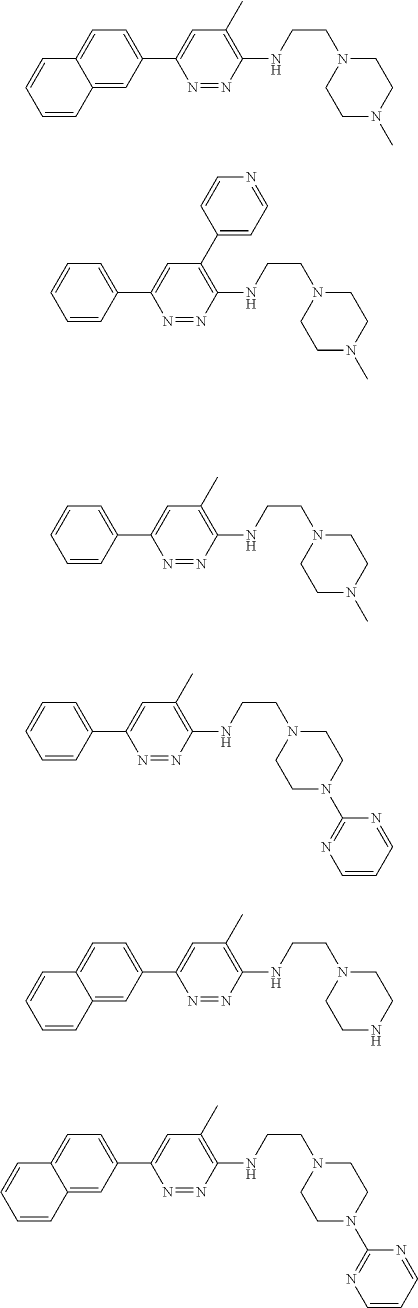 [SMILES: Cc1cc(-c2ccc3ccccc3c2)nnc1NCCN1CCN(C)CC1 ; CN1CCN(CCNc2nnc(-c3ccccc3)cc2-c2ccncc2)CC1 ; Cc1cc(-c2ccccc2)nnc1NCCN1CCN(C)CC1 ; Cc1cc(-c2ccccc2)nnc1NCCN1CCN(c2ncccn2)CC1 ; Cc1cc(-c2ccc3ccccc3c2)nnc1NCCN1CCNCC1 ; Cc1cc(-c2ccc3ccccc3c2)nnc1NCCN1CCN(c2ncccn2)CC1]

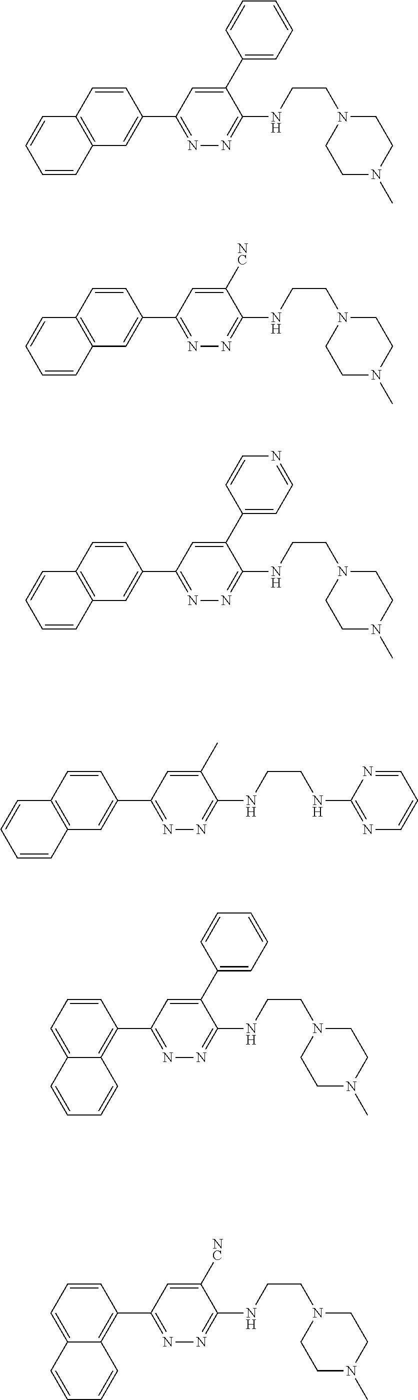

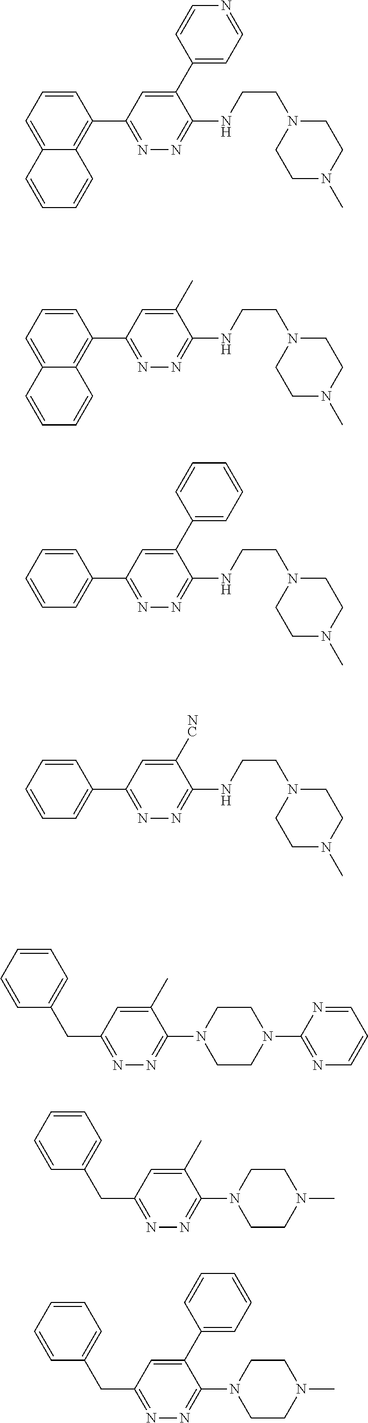

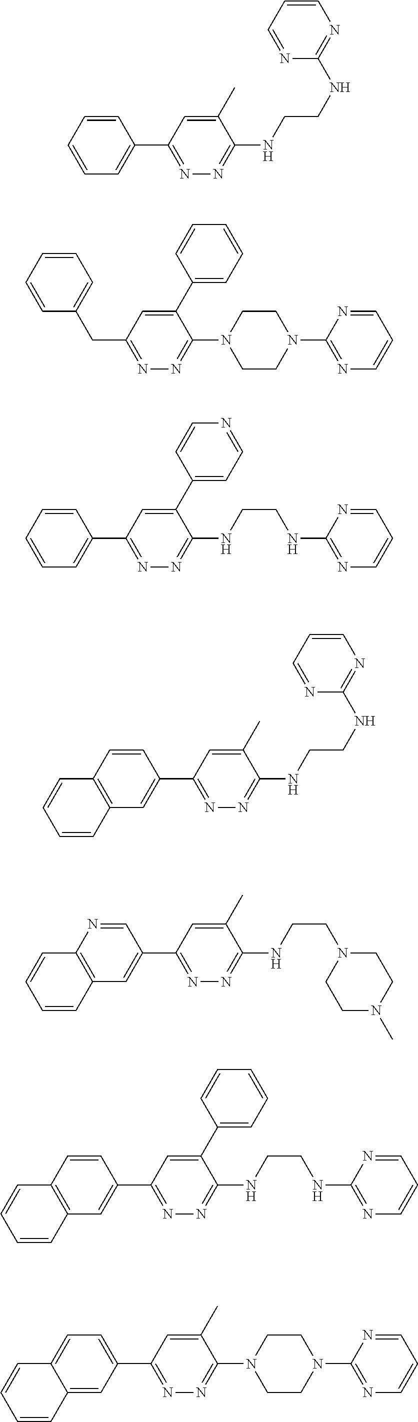

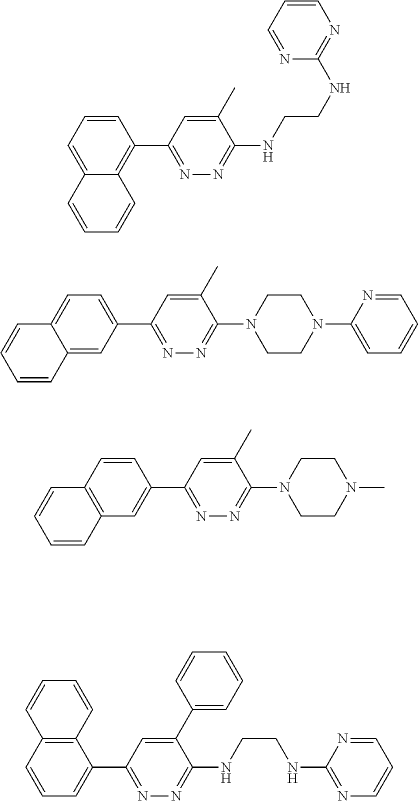 [SMILES: Cc1cc(-c2cccc3ccccc23)nnc1NCCNc1ncccn1 ; Cc1cc(-c2ccc3ccccc3c2)nnc1N1CCN(c2ccccn2)CC1 ; Cc1cc(-c2ccc3ccccc3c2)nnc1N1CCN(C)CC1 ; c1ccc(-c2cc(-c3cccc4ccccc34)nnc2NCCNc2ncccn2)cc1]

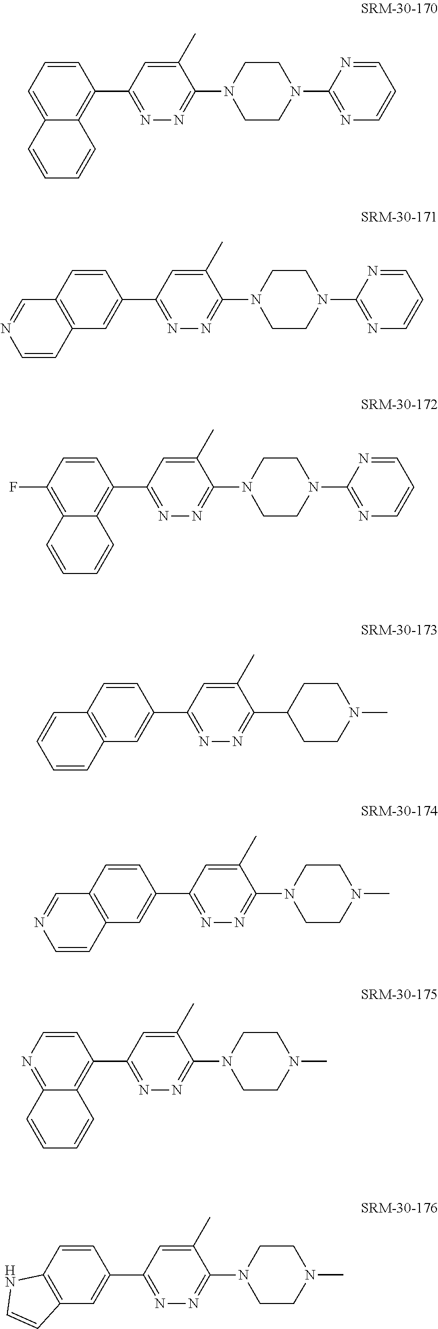

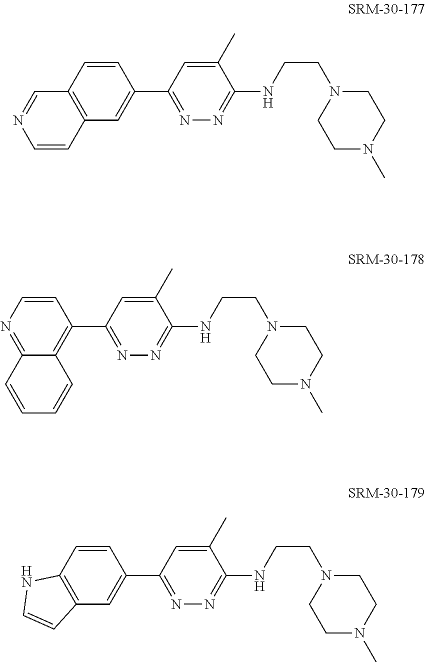 [SMILES: Cc1cc(-c2ccc3cnccc3c2)nnc1NCCN1CCN(C)CC1 ; Cc1cc(-c2ccnc3ccccc23)nnc1NCCN1CCN(C)CC1 ; Cc1cc(-c2ccc3[nH]ccc3c2)nnc1NCCN1CCN(C)CC1]

or a pharmaceutically acceptable salt thereof.

In accordance with one embodiment, the disclosure provides a compound having the following structure:

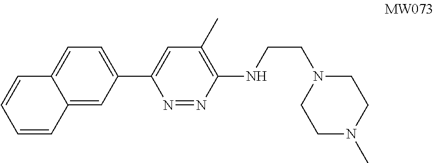 [SMILES: Cc1cc(-c2ccc3ccccc3c2)nnc1NCCN1CCN(C)CC1]

or a pharmaceutically acceptable salt thereof.

In accordance with one embodiment, the disclosure provides a compound having the following structure:

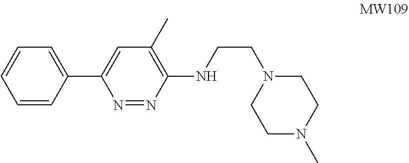 [SMILES: Cc1cc(-c2ccccc2)nnc1NCCN1CCN(C)CC1]

or a pharmaceutically acceptable salt thereof.

In accordance with one embodiment, the disclosure provides a compound of Formula I or Formula II, wherein R₂ is naphthyl or a pharmaceutically acceptable salt thereof.

In accordance with one embodiment, the disclosure provides a compound of Formula I, wherein R₂ is phenyl or naphthyl and R₃, R₄ together with the N to which they are attached form

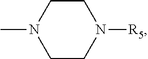

where R₅ is methyl or a pharmaceutically acceptable salt thereof.

In accordance with another aspect, a pharmaceutical composition is provided wherein the composition contains a compound disclosed herein and a pharmaceutically acceptable excipient.

In accordance with one embodiment, the pharmaceutical composition contains a compound having the structure:

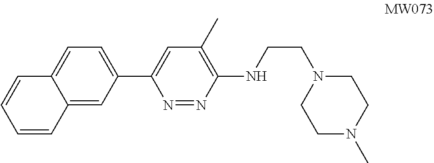 [SMILES: Cc1cc(-c2ccc3ccccc3c2)nnc1NCCN1CCN(C)CC1]

or a pharmaceutically acceptable salt thereof.

In accordance with another aspect, the present disclosure provides a method of treating neuropsychiatric, cognitive or behavioral disorders, especially those associated with neurodegenerative disorders, by administering to a subject a therapeutically effective amount of a compound or pharmaceutical composition disclosed herein.

In accordance with another aspect, the present disclosure provides a method of treating a neurodegenerative disease in a subject in need thereof by administering to the subject a therapeutically effective amount of a compound or pharmaceutical composition disclosed herein.

In accordance with some embodiments, the neurodegenerative disease being treated is Alzheimer's disease, another tauopathy, or dementia.

### BRIEF DESCRIPTION OF THE DRAWINGS

FIG. 1A provides a representative Western Blot of 5HT2bR immunoreactive levels in human brain samples from AD patients and age matched controls. FIG. 1B shows cumulative graphs for control and AD samples.

FIG. 2 is a graph showing efficacy of MW071 in Alzheimer relevant mouse models characterized by Aβ and tau oligomer elevation through their exogenous application using a test of spatial memory, the radial arm water maze (RAWM).

FIGS. 3A-B are graphs showing lack of an effect of MW071 on vision, motility, or motivation in mice infused with Tau or Aβ oligomers. FIG. 3A is a graph of time to reach the visible platform. FIG. 3B is a graph showing average speed.

FIG. 4 is a graph showing efficacy of MW071 in AD relevant mouse models characterized by Aβ and tau oligomer elevation using a test of associative memory, the contextual fear conditioning (FC).

FIG. 5 is a graph showing the lack of an effect of MW071 on cued amygdala dependent memory in mice infused with Tau or Aβ oligomers.

FIGS. 6A-B are graphs showing lack of an effect of MW071 on exploratory behavior in mice infused with Tau or Aβ oligomers. FIG. 6A is a graph illustrating time spent in the center during an open field test among all conditions. FIG. 6B is a graph showing the number of entries into the center during an open field test among all conditions.

FIG. 7 is a graph showing the lack of an effect of MW071 on animal capability of perceiving the electric shock in mice infused with Tau or Aβ oligomers.

FIG. 8 is a graph showing the efficacy of MW071 in AD relevant mouse models characterized by Aβ and tau oligomer elevation using long-term potentiation (LTP) as a test of synaptic plasticity.

FIG. 9 is a graph showing the efficacy of MW073 following intervention at a disease state onto the hTau/Mapt-KO mouse model of tau elevation using a test of spatial memory, the RAWM.

FIG. 10A-B are graphs showing lack of an effect of MW073 on vision, motility, or motivation following intervention at a disease state on the hTau/Mapt-KO mouse model of Alzheimer's disease. FIG. 10A is a graph of the time to reach the visible platform. FIG. 10B is a graph showing the average speed.

FIG. 11 is a graph showing the efficacy of MW073 following intervention at a disease state onto the hTau/Mapt-KO mouse model of tau elevation using a test of associative memory, the contextual fear conditioning.

FIG. 12 is a graph showing the lack of an effect of MW073 on cued amygdala dependent memory following intervention at a disease state onto the hTau/Mapt-KO mouse model of tau elevation.

FIGS. 13A-B are graphs showing the lack of an effect of MW073 on exploratory behavior following intervention at a disease state onto the hTau/Mapt-KO mouse model of tau elevation. FIG. 13A is a graph illustrating time spent in the center during an open field test among all conditions. FIG. 13B is a graph showing the number of entries into the center during an open field test among all conditions.

FIG. 14 is a graph showing the lack of an effect of MW073 on animal capability of perceiving the electric shock following intervention at a disease state onto the hTau/Mapt-KO mouse model of tau elevation.

FIG. 15 is a graph showing efficacy of MW073 in the hTau/Mapt-KO mouse model of tau elevation following intervention at a disease state using LTP as a test of synaptic plasticity.

FIG. 16 is a graph showing the lack of an effect of MW073 on basal neurotransmission in the hTau/Mapt-KO mouse model of tau elevation following intervention at a disease state.

FIG. 17 is a graph showing the efficacy of MW073 in a prevention trial onto the hTau/Mapt-KO mouse model of tau elevation using a test of spatial memory, the RAWM.

FIGS. 18A-B are graphs showing lack of an effect of MW073 on vision, motility, or motivation during a prevention trial on the hTau/Mapt-KO mouse model of Alzheimer's disease. FIG. 18A is a graph of the time to reach the visible platform. FIG. 18B is a graph showing the average speed.

FIG. 19 is a graph showing the efficacy of MW073 in a prevention trial onto the hTau/Mapt-KO mouse model of tau elevation using a test of associative memory, the contextual fear conditioning.

FIG. 20 is a graph showing the lack of an effect of MW073 on cued amygdala dependent memory in a prevention trial onto the hTau/Mapt-KO mouse model of tau elevation.

FIGS. 21A-B are graphs showing the lack of an effect of MW073 on exploratory behavior in a prevention trial onto the hTau/Mapt-KO mouse model of tau elevation. FIG. 21A is a graph illustrating time spent in the center during an open field test among all conditions. FIG. 21B is a graph showing the number of entries into the center during an open field test among all conditions.

FIG. 22 is a graph showing the lack of an effect of MW073 on animal capability of perceiving the electric shock in a prevention trial onto the hTau/Mapt-KO mouse model of tau elevation.

FIG. 23 is a graph showing efficacy of MW073 in the hTau/Mapt-KO mouse model of tau elevation in a prevention trial using LTP as a test of synaptic plasticity.

FIG. 24 is a graph showing the lack of an effect of MW073 on basal neurotransmission in the hTau/Mapt-KO mouse model of tau elevation during a prevention trial.

FIG. 25 is a graph showing the efficacy of MW073 following intervention at a disease state onto the APP/PS1 mouse model of amyloid elevation using a test of spatial memory, the RAWM.

FIGS. 26A-B are graphs showing lack of an effect of MW073 on vision, motility, or motivation following intervention at a disease state onto the APP/PS1 mouse model of amyloid elevation. FIG. 26A is a graph of the time to reach the visible platform. FIG. 26B is a graph showing the average speed.

FIG. 27 is a graph showing the efficacy of MW073 following intervention at a disease state onto the APP/PS1 mouse model of amyloid elevation using a test of associative memory, the contextual fear conditioning.

FIG. 28 is a graph showing the lack of an effect of MW073 on cued amygdala dependent memory following intervention at a disease state onto the APP/PS1 mouse model of amyloid elevation.

FIGS. 29A-B are graphs showing the lack of an effect of MW073 on exploratory behavior following intervention at a disease state onto the APP/PS1 mouse model of amyloid elevation. FIG. 29A is a graph illustrating time spent in the center during an open field test among all conditions. FIG. 29B is a graph showing the number of entries into the center during an open field test among all conditions.

FIG. 30 is a graph showing the lack of an effect of MW073 on animal capability of perceiving the electric shock following intervention at a disease state onto the APP/PS1 mouse model of amyloid elevation.

FIG. 31 is a graph showing efficacy of MW073 onto the APP/PS1 mouse model of amyloid elevation following intervention at a disease state using LTP as a test of synaptic plasticity.

FIG. 32 is a graph showing the lack of an effect of MW073 on basal neurotransmission in the APP/PS1 mouse model of amyloid elevation following intervention at a disease state.

FIG. 33 is a graph showing the efficacy of MW073 in a prevention trial onto the APP/PS1 mouse model of amyloid elevation using a test of spatial memory, the RAWM.

FIGS. 34A-B are graphs showing lack of an effect of MW073 on vision, motility, or motivation during a prevention trial onto the APP/PS1 mouse model of amyloid elevation. FIG. 34A is a graph of the time to reach the visible platform. FIG. 34B is a graph showing the average speed.

FIG. 35 is a graph showing the efficacy of MW073 in a prevention trial onto the APP/PS1 mouse model of amyloid elevation using a test of associative memory, the contextual fear conditioning.

FIG. 36 is a graph showing the lack of an effect of MW073 on cued amygdala dependent memory in a prevention trial onto the APP/PS1 mouse model of amyloid elevation.

FIGS. 37A-B are graphs showing the lack of an effect of MW073 on exploratory behavior in a prevention trial onto the APP/PS1 mouse model of amyloid elevation. FIG. 37A is a graph illustrating time spent in the center during an open field test among all conditions. FIG. 37B is a graph showing the number of entries into the center during an open field test among all conditions.

FIG. 38 is a graph showing the lack of an effect of MW073 on animal capability of perceiving the electric shock in a prevention trial onto the APP/PS1 mouse model of amyloid elevation.

FIG. 39 is a graph showing efficacy of MW073 onto the APP/PS1 mouse model of amyloid elevation in a prevention trial using LTP as a test of synaptic plasticity.

FIG. 40 is a graph showing the lack of an effect of MW073 on basal neurotransmission in the APP/PS1 mouse model of amyloid elevation during a prevention trial.

FIG. 41 is a graph showing the efficacy of MW073 in AD relevant mouse models characterized by Aβ and tau oligomer elevation using long-term potentiation (LTP) as a test of synaptic plasticity.

FIGS. 42A-B are graphs showing dose response curve for the beneficial effect of MW073 onto radial arm water maze (RAWM) and contextual fear conditioning (FC) defects in APP/PS1 mice. The values of ED50s are indicated in the graphs. FIG. 42A is a graph illustrating the dose response curve and ED50 with the RAWM. FIG. 42B is a graph illustrating the dose response curve and ED50 with the FC.

FIG. 43 is a graph showing the efficacy of MW109 following intervention at a disease state onto the hTau/Mapt-KO mouse model of tau elevation using a test of spatial memory, the RAWM.

FIGS. 44A-B are graphs showing lack of an effect of MW109 on vision, motility, or motivation following intervention at a disease state on the hTau/Mapt-KO mouse model of Alzheimer's disease. FIG. 44A is a graph of the time to reach the visible platform. FIG. 44B is a graph showing the average speed.

FIG. 45 is a graph showing the efficacy of MW109 following intervention at a disease state onto the hTau/Mapt-KO mouse model of tau elevation using a test of associative memory, the contextual fear conditioning.

FIG. 46 is a graph showing the lack of an effect of MW109 on cued amygdala dependent memory following intervention at a disease state onto the hTau/Mapt-KO mouse model of tau elevation.

FIGS. 47A-B are graphs showing the lack of an effect of MW109 on exploratory behavior following intervention at a disease state onto the hTau/Mapt-KO mouse model of tau elevation. FIG. 47A is a graph illustrating time spent in the center during an open field test among all conditions. FIG. 47B is a graph showing the number of entries into the center during an open field test among all conditions.

FIG. 48 is a graph showing the lack of an effect of MW109 on animal capability of perceiving the electric shock following intervention at a disease state onto the hTau/Mapt-KO mouse model of tau elevation.

FIG. 49 is a graph showing efficacy of MW109 in the hTau/Mapt-KO mouse model of tau elevation following intervention at a disease state using LTP as a test of synaptic plasticity.

FIG. 50 is a graph showing the lack of an effect of MW109 on basal neurotransmission in the hTau/Mapt-KO mouse model of tau elevation following intervention at a disease state.

FIG. 51 is a graph showing the efficacy of MW109 in a prevention trial onto the hTau/Mapt-KO mouse model of tau elevation using a test of spatial memory, the RAWM.

FIGS. 52A-B are graphs showing lack of an effect of MW109 on vision, motility, or motivation during a prevention trial on the hTau/Mapt-KO mouse model of Alzheimer's disease. FIG. 52A is a graph of the time to reach the visible platform. FIG. 52B is a graph showing the average speed.

FIG. 53 is a graph showing the efficacy of MW109 in a prevention trial onto the hTau/Mapt-KO mouse model of tau elevation using a test of associative memory, the contextual fear conditioning.

FIG. 54 is a graph showing the lack of an effect of MW109 on cued amygdala dependent memory in a prevention trial onto the hTau/Mapt-KO mouse model of tau elevation.

FIGS. 55A-B are graphs showing the lack of an effect of MW109 on exploratory behavior in a prevention trial onto the hTau/Mapt-KO mouse model of tau elevation. FIG. 55A is a graph illustrating time spent in the center during an open field test among all conditions. FIG. 55B is a graph showing the number of entries into the center during an open field test among all conditions.

FIG. 56 is a graph showing the lack of an effect of MW109 on animal capability of perceiving the electric shock in a prevention trial onto the hTau/Mapt-KO mouse model of tau elevation.

FIG. 57 is a graph showing efficacy of MW109 in the hTau/Mapt-KO mouse model of tau elevation in a prevention trial using LTP as a test of synaptic plasticity.

FIG. 58 is a graph showing the lack of an effect of MW109 on basal neurotransmission in the hTau/Mapt-KO mouse model of tau elevation during a prevention trial.

FIG. 59 is a graph showing the efficacy of MW109 following intervention at a disease state onto the APP/PS1 mouse model of amyloid elevation using a test of spatial memory, the RAWM.

FIGS. 60A-B are graphs showing lack of an effect of MW109 on vision, motility, or motivation following intervention at a disease state onto the APP/PS1 mouse model of amyloid elevation. FIG. 60A is a graph of the time to reach the visible platform. FIG. 60B is a graph showing the average speed.

FIG. 61 is a graph showing the efficacy of MW109 following intervention at a disease state onto the APP/PS1 mouse model of amyloid elevation using a test of associative memory, the contextual fear conditioning.

FIG. 62 is a graph showing the lack of an effect of MW109 on cued amygdala dependent memory following intervention at a disease state onto the APP/PS1 mouse model of amyloid elevation.

FIGS. 63A-B are graphs showing the lack of an effect of MW109 on exploratory behavior following intervention at a disease state onto the APP/PS1 mouse model of amyloid elevation. FIG. 63A is a graph illustrating time spent in the center during an open field test among all conditions. FIG. 63B is a graph showing the number of entries into the center during an open field test among all conditions.

FIG. 64 is a graph showing the lack of an effect of MW109 on animal capability of perceiving the electric shock following intervention at a disease state onto the APP/PS1 mouse model of amyloid elevation.

FIG. 65 is a graph showing efficacy of MW109 onto the APP/PS1 mouse model of amyloid elevation following intervention at a disease state using LTP as a test of synaptic plasticity.

FIG. 66 is a graph showing the lack of an effect of MW109 on basal neurotransmission in the APP/PS1 mouse model of amyloid elevation following intervention at a disease state.

FIG. 67 is a graph showing the efficacy of MW109 in a prevention trial onto the APP/PS1 mouse model of amyloid elevation using a test of spatial memory, the RAWM.

FIGS. 68A-B are graphs showing lack of an effect of MW109 on vision, motility, or motivation during a prevention trial onto the APP/PS1 mouse model of amyloid elevation. FIG. 68A is a graph of the time to reach the visible platform. FIG. 68B is a graph showing the average speed.

FIG. 69 is a graph showing the efficacy of MW109 in a prevention trial onto the APP/PS1 mouse model of amyloid elevation using a test of associative memory, the contextual fear conditioning.

FIG. 70 is a graph showing the lack of an effect of MW109 on cued amygdala dependent memory in a prevention trial onto the APP/PS1 mouse model of amyloid elevation.

FIGS. 71A-B are graphs showing the lack of an effect of MW109 on exploratory behavior in a prevention trial onto the APP/PS1 mouse model of amyloid elevation. FIG. 71A is a graph illustrating time spent in the center during an open field test among all conditions. FIG. 71B is a graph showing the number of entries into the center during an open field test among all conditions.

FIG. 72 is a graph showing the lack of an effect of MW109 on animal capability of perceiving the electric shock in a prevention trial onto the APP/PS1 mouse model of amyloid elevation.

FIG. 73 is a graph showing efficacy of MW109 onto the APP/PS1 mouse model of amyloid elevation in a prevention trial using LTP as a test of synaptic plasticity.

FIG. 74 is a graph showing the lack of an effect of MW109 on basal neurotransmission in the APP/PS1 mouse model of amyloid elevation during a prevention trial.

FIG. 75 is a graph showing efficacy of MW109 in Alzheimer relevant mouse models characterized by Aβ and tau oligomer elevation through their exogenous application using a test of spatial memory, the radial arm water maze (RAWM).

FIGS. 76A-B are graphs showing lack of an effect of MW109 on vision, motility, or motivation in mice infused with Tau or Aβ oligomers. FIG. 76A is a graph of time to reach the visible platform. FIG. 76B is a graph showing average speed.

FIG. 77 is a graph showing efficacy of MW109 in AD relevant mouse models characterized by Aβ and tau oligomer elevation using a test of associative memory, the contextual fear conditioning (FC).

FIG. 78 is a graph showing the lack of an effect of MW109 on cued amygdala dependent memory in mice infused with Tau or Aβ oligomers.

FIGS. 79A-B are graphs showing lack of an effect of MW109 on exploratory behavior in mice infused with Tau or Aβ oligomers. FIG. 79A is a graph illustrating time spent in the center during an open field test among all conditions. FIG. 79B is a graph showing the number of entries into the center during an open field test among all conditions.

FIG. 80 is a graph showing the lack of an effect of MW109 on animal capability of perceiving the electric shock in mice infused with Tau or Aβ oligomers.

FIG. 81 is a graph showing the efficacy of MW109 in AD relevant mouse models characterized by Aβ and tau oligomer elevation using long-term potentiation (LTP) as a test of synaptic plasticity.

FIG. 82 is a graph illustrating a dose-response curve with ED50 for the beneficial effect of MW109 onto the long-term potentiation (LTP) defect in hippocampal slices perfused with oligomeric tau.

FIG. 83 is a graph illustrating a dose-response curve for the beneficial effect of MW109 onto the long-term potentiation (LTP) defect in hippocampal slices perfused with oligomeric tau.

FIG. 84 is a graph showing a phenotypical screening of different small molecules (minaprine, MW071, MW073, and MW109) based on the ability to rescue the detrimental effect of 50 nM oligomeric tau onto long-term potentiation (LTP) in slices from the hippocampus of C57Bl6 mice when co-administered at 1.9 μM for 20 min (the ED50 for MW109).

FIGS. 85A-B provide graphs showing that Tg2576 males are more aggressive than Tg2576 females. The bar graphs represent rate scores related to aggressive behavior in Tg2576 resident males and Tg2576 resident females. FIG. 85A is a bar graph showing the number of attacks and FIG. 85B is a bar graph showing the total attack time.

FIGS. 86A-D provide bar graphs showing Tg2576 resident males are more aggressive than nTg resident males. The bar graphs represent rate scores related to aggressive behavior in Tg2576 and non-transgenic (nTg) resident males. FIG. 86A is a bar graph showing that nTg residents displayed longer latency to the first attack compared to Tg2576 mice. FIG. 86B shows slightly higher but not statistically significant duration of the first attack in Tg2576 residents compared to nTg residents (nTg=4.33±1.20 sec, Tg=5.909±1.74 sec, p=0.6590). FIG. 86C-D shows a dramatic increase in the number of attacks, as well as the total attack time in Tg animals compared to nTg mice.

FIGS. 87A-D shows that Tg2576 mice treated with MW073 showed amelioration of aggressive behavior. Bar graphs representing rate scores related to aggressive behavior in Tg2576 mice treated with vehicle and Tg2576 treated with MW073. FIG. 87A shows that Tg2576 resident males treated with MW073 displayed a slightly longer but not statistically different latency to the first attack compared to Tg2576 treated with vehicle. FIG. 87B shows that Tg2576 resident males treated with MW073 showed a decrease in the duration of the first attack. FIGS. 87C-D show that the number of attacks, as well as the total attack time, of Tg2576 resident males treated with MW073 was reduced compared to Tg2576 treated with vehicle.

FIG. 88 illustrates the experimental design for the tests relating to aggressive behavior.

### DETAILED DESCRIPTION

The present application discloses compounds and pharmaceutical compositions that may be used for treating cognitive dysfunction and neuropsychiatric conditions associated with neurodegenerative disorders, such as Alzheimer's Disease, other tauopathies, and dementia.

In the following description, certain specific details are set forth in order to provide a thorough understanding of various embodiments. However, one skilled in the art will understand that the invention can be practiced without these details. In other instances, well-known structures have not been shown or described in detail to avoid unnecessarily obscuring descriptions of the embodiments.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

“Aryl” refers to a hydrocarbon ring system radical comprising hydrogen, 5 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in this disclosure, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂ OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, a “subject” can be a human, non-human primate, mammal, rat, mouse, cow, horse, pig, sheep, goat, dog, cat, insect and the like. The subject can be suspected of having or at risk for AD, tauopathy, dementia or another disease or condition. Diagnostic methods for these conditions are known to those of ordinary skill in the art.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, increased life span, increased life expectancy or prevention of the progression of the disease or condition. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, preferably a human, having the disease or condition of interest, and includes (but is not limited to):
a) preventing the disease or condition from occurring in a mammal, in particular, when such mammal is predisposed to the condition but has not yet been diagnosed as having it;
b) inhibiting the disease or condition, e.g., arresting its development;
c) relieving the disease or condition, e.g., causing regression of the disease or condition (ranging from reducing the severity of the disease or condition to curing the disease of condition); or
d) relieving the symptoms resulting from the disease or condition, e.g., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values herein. Furthermore, the phrases “less than about [a value]” or “greater than about [a value]” should be understood in view of the definition of the term “about” provided herein. The terms “about” and “approximately” can be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Following below are more detailed descriptions of various concepts related to, and embodiments of inventive compounds and methods for the treatment of neurodegenerative diseases. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### Compounds

In accordance with one aspect, a compound of Formula (I):

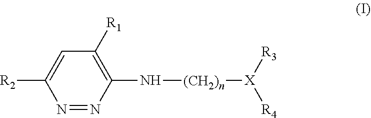

wherein R₁ is CN, C₁-C₆ alkyl, aryl, or heteroaryl; R₂ is phenyl, benzyl, naphthyl, quinolinyl, isoquinolinyl, or indolyl, wherein said phenyl, benzyl, naphthyl, quinolinyl, isoquinolinyl or indolyl is optionally independently substituted with at least one halogen; X is CH or N; n is 1 or 2; and R₃ and R₄ are independently H, C₁-C₆ alkyl, aryl or heteroaryl, provided that R₃ and R₄ are not both H, or R₃, R₄ together with the X to which they are attached form

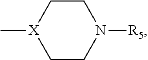

where R₅ is H, C₁-C₆ alkyl, aryl, or heteroaryl; or a pharmaceutically acceptable salt thereof.

In one embodiment, the present application is directed to compounds of Formula (Ia):

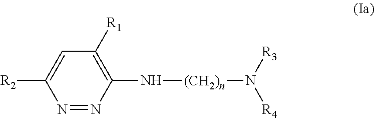

wherein
R₁ is CN, C₁-C₆ alkyl, aryl, or heteroaryl;
R₂ is phenyl, benzyl, naphthyl, quinolinyl, isoquinolinyl, or indolyl, wherein said phenyl, benzyl, naphthyl, quinolinyl, isoquinolinyl or indolyl is optionally independently substituted with at least one halogen;
n is 1 or 2; and
R₃ and R₄ are independently H, C₁-C₆ alkyl, aryl or heteroaryl or R₃, R₄ together with the N to which they are attached form

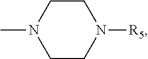

where R₅ is H, C₁-C₆ alkyl, aryl, or heteroaryl.

In some embodiments, compounds of Formula (I) or (Ia) are disclosed wherein R₁ is CN, C₁-C₂ alkyl, phenyl, or pyridyl;
R₂ is phenyl, or naphthyl;
n is 2; and
R₃, R₄ together with the N to which they are attached form

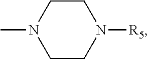

where R₅ is H, C₁-C₂ alkyl, aryl, or heteroaryl containing at least one N in an aryl ring.

In some embodiments, compounds of Formula (I) or (Ia) are disclosed wherein R₁ is methyl;
R₂ is phenyl, or naphthyl;
n is 2; and
R₃, R₄ together with the N to which they are attached form

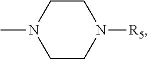

where R₅ is C₁-C₂ alkyl.

In another aspect, the present application is directed to compounds of Formula (II):

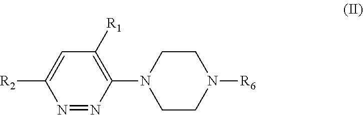

wherein
R₁ is CN, C₁-C₆ alkyl, aryl, or heteroaryl;
R₂ is benzyl, naphthyl, quinolinyl, isoquinolinyl, or indolyl, wherein said benzyl, naphthyl, quinolinyl, isoquinolinyl or indolyl is optionally independently substituted with at least one halogen; and
R₆ is H, C₁-C₆ alkyl, aryl, or heteroaryl; or a pharmaceutically acceptable salt thereof.

In some embodiments, compounds of Formula (II) are disclosed, wherein R₁ is CN, C₁-C₂ alkyl, phenyl, or pyridyl and R₆ is C₁-C₂ alkyl, or heteroaryl containing at least one N in the aryl ring; or a pharmaceutically acceptable salt thereof.

In some embodiments, compounds of Formula (II) are disclosed, wherein R₁ is methyl, CN, C₁-C₂ alkyl, phenyl, or pyridyl; and
R₆ is methyl, pyridinyl or pyrimidinyl; or a pharmaceutically acceptable salt thereof.

In some embodiments, Formula (I) excludes compounds having the structure:

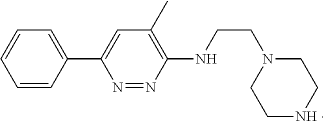 [SMILES: Cc1cc(-c2ccccc2)nnc1NCCN1CCNCC1]

In some embodiments of formula (I) or Formula (II), R₂ is naphthalen-1-yl, naphthalen-2-yl, quinolin-8-yl, or isoquinolin-5-yl, wherein naphthalen-1-yl, naphthalen-2-yl, quinolin-8-yl, or isoquinolin-5-yl is optionally independently substituted with at least one halogen atom. In some embodiments, the halogen atom is chlorine or fluorine. In some embodiments, the halogen atom is chlorine. In some embodiments, the halogen atom is fluorine.

In some embodiments of Formula (I) or Formula (II), the compounds have a structure as in Table I, below, or a pharmaceutically acceptable salt or solvate thereof. Table I provides Serotonin 5-HT2bR binding activity, synaptic in situ activity and in vivo function for various compounds.

TABLE I

| | | 5HT2bR | | |
|---|---|---|---|---|
| | | Binding* | Synaptic | In vivo |
| Structure | Name | IC₅₀ (nM) | Activity | Behavior |
| 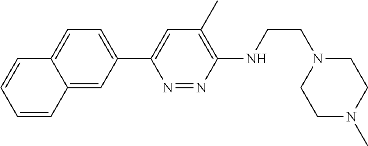 [SMILES: Cc1cc(-c2ccc3ccccc3c2)nnc1NCCN1CCN(C)CC1] | MW073 | 70 ± 26 | YES | YES |
| 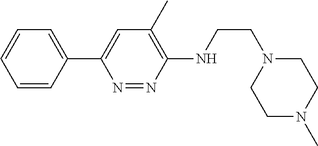 [SMILES: Cc1cc(-c2ccccc2)nnc1NCCN1CCN(C)CC1] | MW109 | 593 ± 162 | YES | YES |
| 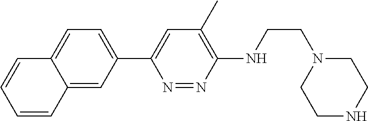 [SMILES: Cc1cc(-c2ccc3ccccc3c2)nnc1NCCN1CCNCC1] | MW046 | 174 ± 85.6 | | |
| 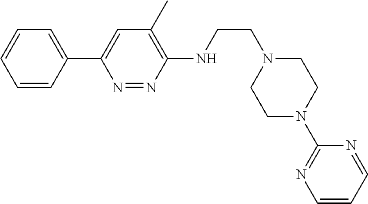 [SMILES: Cc1cc(-c2ccccc2)nnc1NCCN1CCN(c2ncccn2)CC1] | MW106 | 99 ± 9.4 | | |
| 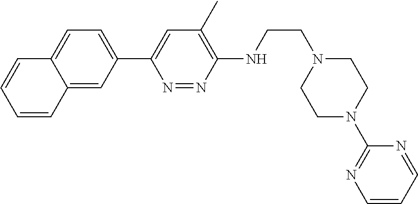 [SMILES: Cc1cc(-c2ccc3ccccc3c2)nnc1NCCN1CCN(c2ncccn2)CC1] | 119.8 ± 37.6 | | | |
| 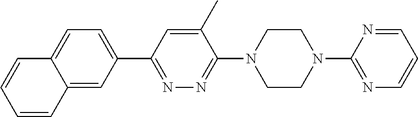 [SMILES: Cc1cc(-c2ccc3ccccc3c2)nnc1N1CCN(c2ncccn2)CC1] | MW024 | 85 ± 14 | | |
| 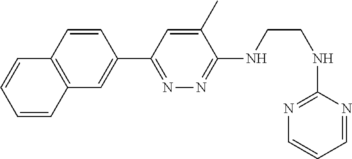 [SMILES: Cc1cc(-c2ccc3ccccc3c2)nnc1NCCNc1ncccn1] | MW030 | 187 ± 48 | | |
| 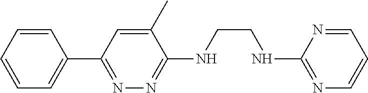 [SMILES: Cc1cc(-c2ccccc2)nnc1NCCNc1ncccn1] | MW078 | 770 ± 127 | | |
| 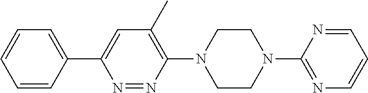 [SMILES: Cc1cc(-c2ccccc2)nnc1N1CCN(c2ncccn2)CC1] | MW151 | 762 ± 235 | | |
| 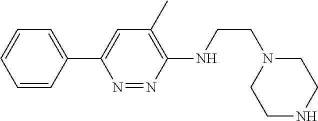 [SMILES: Cc1cc(-c2ccccc2)nnc1NCCN1CCNCC1] | MW071 | 25 ± 7 | | |
| 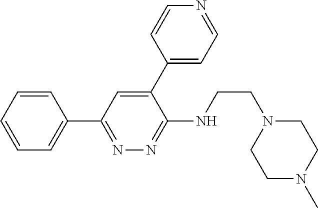 [SMILES: CN1CCN(CCNc2nnc(-c3ccccc3)cc2-c2ccncc2)CC1] | MW033 | N.C.: IC50 value not calculable. Concentration- response curve shows less than 25% effect at the highest validating testing concentration. | | |
| 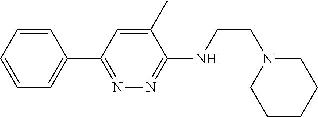 [SMILES: Cc1cc(-c2ccccc2)nnc1NCCN1CCCCC1] | MW103 | 83 | | |
| 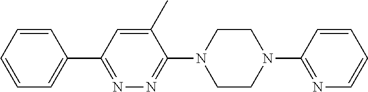 [SMILES: Cc1cc(-c2ccccc2)nnc1N1CCN(c2ccccn2)CC1] | MW081 | 730 ± 70.7 | | |
| 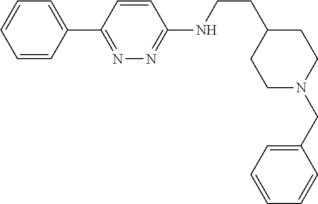 [SMILES: c1ccc(CN2CCC(CCNc3ccc(-c4ccccc4)nn3)CC2)cc1] | MW041 | 450 ± 113 | | |

*Compounds tested over a concentration range from 10,000 nM to 1.0 nM

In some embodiments of Formula (I) or Formula (II), the compounds have a structure as provided, below, or a pharmaceutically acceptable salt or solvate thereof.

| Structure | Name |
|---|---|
| 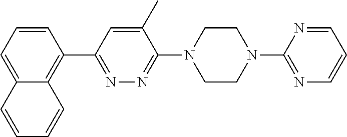 [SMILES: Cc1cc(-c2cccc3ccccc23)nnc1N1CCN(c2ncccn2)CC1] | SRM- 30-170 |
| 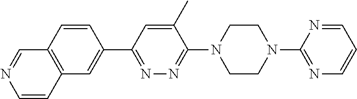 [SMILES: Cc1cc(-c2ccc3cnccc3c2)nnc1N1CCN(c2ncccn2)CC1] | SRM- 30-171 |
| 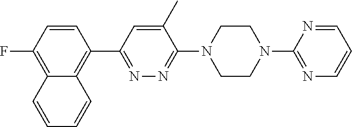 [SMILES: Cc1cc(-c2ccc(F)c3ccccc23)nnc1N1CCN(c2ncccn2)CC1] | SRM- 30-172 |
| 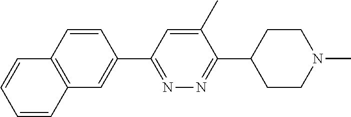 [SMILES: Cc1cc(-c2ccc3ccccc3c2)nnc1C1CCN(C)CC1] | SRM- 30-173 |
| 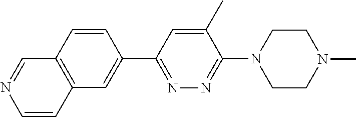 [SMILES: Cc1cc(-c2ccc3cnccc3c2)nnc1N1CCN(C)CC1] | SRM- 30-174 |
| 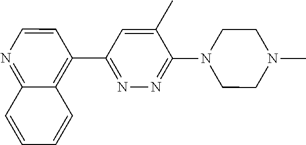 [SMILES: Cc1cc(-c2ccnc3ccccc23)nnc1N1CCN(C)CC1] | SRM- 30-175 |
| 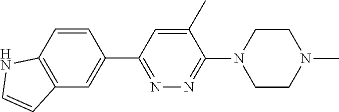 [SMILES: Cc1cc(-c2ccc3[nH]ccc3c2)nnc1N1CCN(C)CC1] | SRM- 30-176 |
| 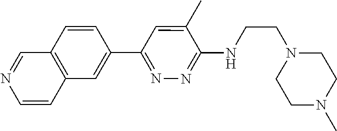 [SMILES: Cc1cc(-c2ccc3cnccc3c2)nnc1NCCN1CCN(C)CC1] | SRM- 30-177 |
| 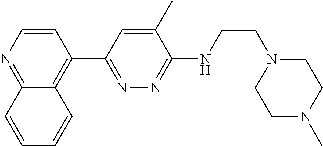 [SMILES: Cc1cc(-c2ccnc3ccccc23)nnc1NCCN1CCN(C)CC1] | SRM- 30-178 |
| 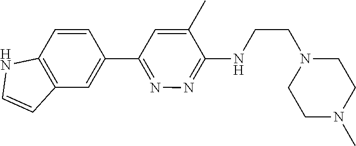 [SMILES: Cc1cc(-c2ccc3[nH]ccc3c2)nnc1NCCN1CCN(C)CC1] | SRM- 30-179 |
| 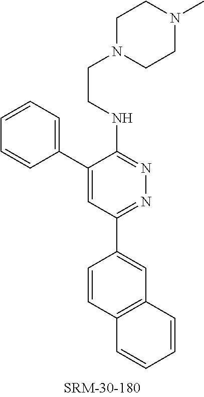 [SMILES: CN1CCN(CCNc2nnc(-c3ccc4ccccc4c3)cc2-c2ccccc2)CC1] | SRM- 30-180 |
| 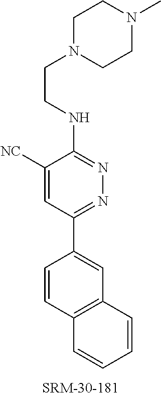 [SMILES: CN1CCN(CCNc2nnc(-c3ccc4ccccc4c3)cc2C#N)CC1] | SRM- 30-181 |
| 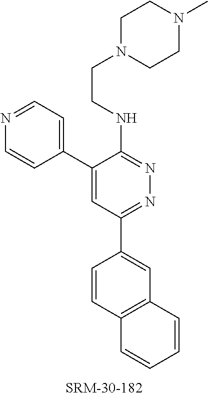 [SMILES: CN1CCN(CCNc2nnc(-c3ccc4ccccc4c3)cc2-c2ccncc2)CC1] | SRM- 30-182 |
| 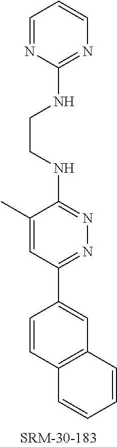 [SMILES: Cc1cc(-c2ccc3ccccc3c2)nnc1NCCNc1ncccn1] | SRM- 30-183 |
| 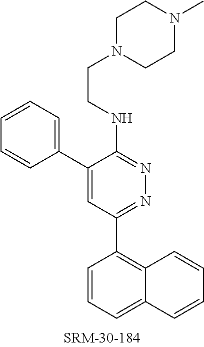 [SMILES: CN1CCN(CCNc2nnc(-c3cccc4ccccc34)cc2-c2ccccc2)CC1] | SRM- 30-184 |
| 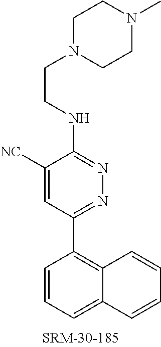 [SMILES: CN1CCN(CCNc2nnc(-c3cccc4ccccc34)cc2C#N)CC1] | SRM- 30-185 |
| 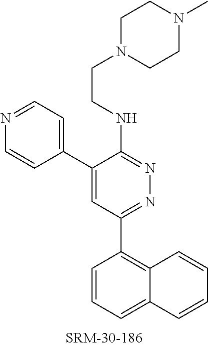 [SMILES: CN1CCN(CCNc2nnc(-c3cccc4ccccc34)cc2-c2ccncc2)CC1] | SRM- 30-186 |
| 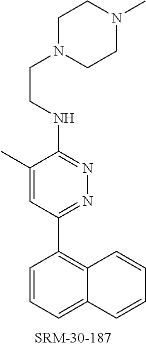 [SMILES: Cc1cc(-c2cccc3ccccc23)nnc1NCCN1CCN(C)CC1] | SRM- 30-187 |
| 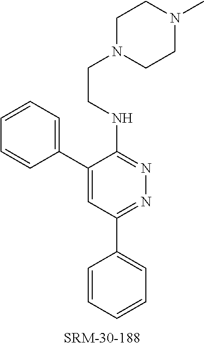 [SMILES: CN1CCN(CCNc2nnc(-c3ccccc3)cc2-c2ccccc2)CC1] | SRM- 30-188 |
| 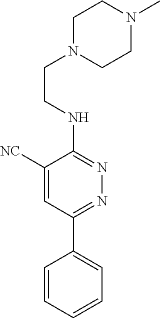 [SMILES: CN1CCN(CCNc2nnc(-c3ccccc3)cc2C#N)CC1] | SRM- 30-189 |
| 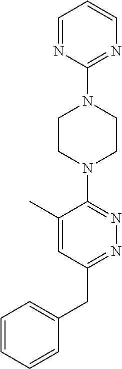 [SMILES: Cc1cc(Cc2ccccc2)nnc1N1CCN(c2ncccn2)CC1] | SRM- 30-190 |
| 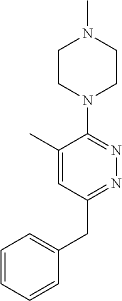 [SMILES: Cc1cc(Cc2ccccc2)nnc1N1CCN(C)CC1] | SRM- 30-191 |
| 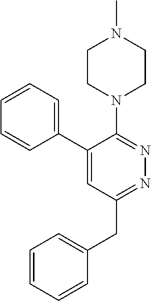 [SMILES: CN1CCN(c2nnc(Cc3ccccc3)cc2-c2ccccc2)CC1] | SRM- 30-192 |
| 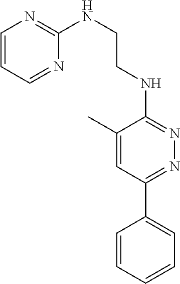 [SMILES: Cc1cc(-c2ccccc2)nnc1NCCNc1ncccn1] | SRM- 30-193 |
| 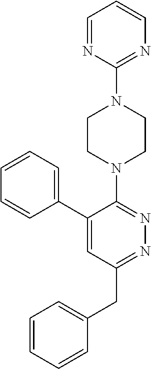 [SMILES: c1ccc(Cc2cc(-c3ccccc3)c(N3CCN(c4ncccn4)CC3)nn2)cc1] | SRM- 30-194 |
| 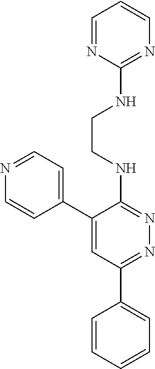 [SMILES: c1ccc(-c2cc(-c3ccncc3)c(NCCNc3ncccn3)nn2)cc1] | SRM- 30-195 |
| 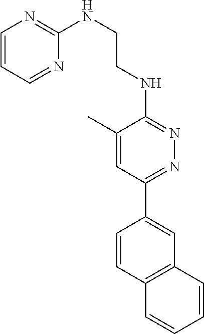 [SMILES: Cc1cc(-c2ccc3ccccc3c2)nnc1NCCNc1ncccn1] | SRM- 30-196 |
| 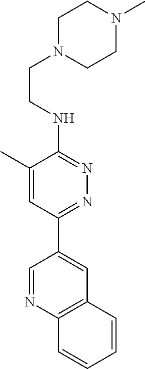 | SRM- 30-197 |
| 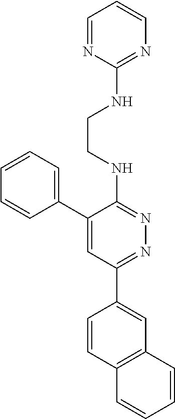 [SMILES: c1ccc(-c2cc(-c3ccc4ccccc4c3)nnc2NCCNc2ncccn2)cc1] | SRM- 30-198 |
| 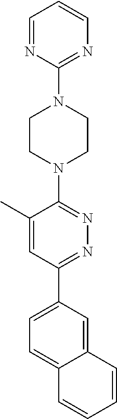 [SMILES: Cc1cc(-c2ccc3ccccc3c2)nnc1N1CCN(c2ncccn2)CC1] | SRM- 30-199 |
| 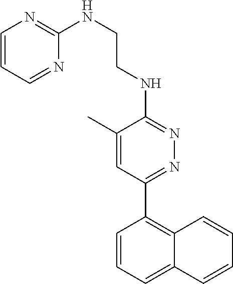 [SMILES: Cc1cc(-c2cccc3ccccc23)nnc1NCCNc1ncccn1] | SRM- 30-200 |
| 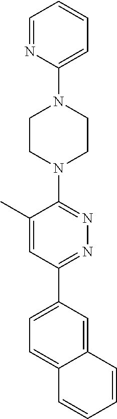 [SMILES: Cc1cc(-c2ccc3ccccc3c2)nnc1N1CCN(c2ccccn2)CC1] | SRM- 30-201 |
| 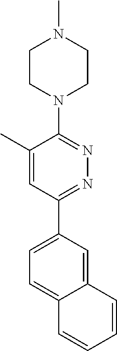 [SMILES: Cc1cc(-c2ccc3ccccc3c2)nnc1N1CCN(C)CC1] | SRM- 30-202 |
| 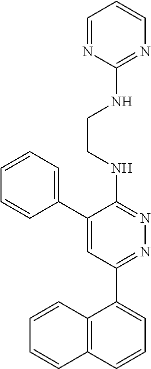 [SMILES: c1ccc(-c2cc(-c3cccc4ccccc34)nnc2NCCNc2ncccn2)cc1] | SRM- 30-203 |

### Pharmaceutical Compositions

In some embodiments, the present disclosure provides pharmaceutical compositions comprising an effective amount of a compound disclosed herein or a pharmaceutically acceptable salt thereof. In some embodiments, the pharmaceutical compositions provided herein comprise one or more pharmaceutically acceptable carriers or excipients.

In various embodiments, the pharmaceutical compositions of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The effective amount of a compound of Formula (I) or (II), pharmaceutically acceptable salts, esters, prodrugs, hydrates, solvates and isomers thereof, or a pharmaceutical composition comprising a compound of Formula (I) or (II) or a pharmaceutically acceptable salt thereof may be determined by one skilled in the art based on known methods.

In one embodiment, a pharmaceutical composition or a pharmaceutical formulation of the present disclosure comprises a compound of Formula (I) or (II) or a pharmaceutically acceptable salt thereof, and a pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, preferably in physiologically compatible buffers such as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

### Methods of Use

In some embodiments, the compounds disclosed herein can be used to treat a neurodegenerative disease in a subject in need thereof. In some embodiments, the neurodegenerative disease may be Alzheimer's disease, or tauopathy, or Alzheimer's Disease related dementia.

In some embodiments, the compounds have high selectivity, reasonable pharmacokinetics and/or good permeability across the blood-brain-barrier (BBB). In some embodiments, these compounds can be used as therapy with decreased side effects for Alzheimer's Disease patients. In some embodiments, the compounds improve cognition or memory in Alzheimer's Disease, tauopathies, and Alzheimer's-disease related dementia, as well as minimize the side effects for subjects afflicted with other neurodegenerative diseases.

In some embodiments, the subject is afflicted with Alzheimer's disease, or other tauopathies, or Alzheimer's disease related dementia.

In some embodiments, the compounds disclosed herein can be used to treat a neuropsychiatric disease in a subject in need thereof. In some embodiments, the neuropsychiatric disease may be a depressive disorder, mood disorder, anxiety disorder, behavioral disorder or cognitive disorder. In some embodiments, the compounds disclosed herein can be used to prevent, reduce or treat aggressivity, more particularly aggressivity associated with a neurodegenerative disease.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Synthesis of Compounds

### Synthesis of MW071

### Chemical Name: 4-methyl-6-phenyl-N-(2-(piperazin-1-yl)ethyl)pyridazin-3-amine

Compound 1 (200 mg, 0.98 mmol) and 4-N-(2-aminoethyl)-N-Boc-piperazine (6 eq, 3.91 mmol, 896 mg) were reacted in 1-butanol. The temperature was held at 145° C. for ˜26 h the reaction was worked up by adding H₂O (50 mL), extracted with CH₂Cl₂ (3×15 mL) and dried with MgSO₄. Column purification on silica gel using 5% MeOH/95% CH₂Cl₂ a yellowish oil compound 2 (tert-butyl 4-(2-((4-methyl-6-phenylpyridazin-3-yl)amino)ethyl)piperazine-1-carboxylate), yield 82%, ESI, 398.25 (MH+) confirmed the Boc-compound. The intermediate Boc-compound 2 was treated with formic acid in CH₂Cl₂ for ˜5 h at ambient temperature to yield compound MW071. After workup product was purified on column chromatography, with the product eluting with 5% (v/v) methanol in methylene chloride yielded MW071, 96% pure, yellow powder in 80% yield.

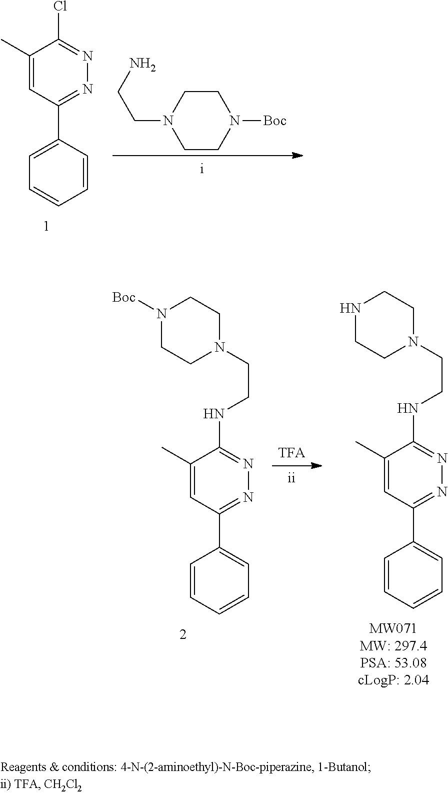

### Analytical:

HIPLC: HPLC (tr/purity): 8.93 min, 97%

NMR: ¹H NMR (CD₃OD): δ 8.28 (s, 1H); 7.94 (d, J=7 Hz, 2H); 7.67 (m, 3H); 4.05 (t, J=5 Hz, 2H); 3.76 (bs, 4H); 3.70 (t, J=5 Hz, 4H); 3.31 (s, 2H); 2.60 (s, 3H); 2.02 (s, 1H)

ESI (m/z): 298.31 (MH+).

HRMS: HRMS calculated for C₁₇H₂₄N₅ 298.2026, found 298.2025

### General Synthesis Scheme for MW073:

Chemical Formula: C₂₂H₂₇N₅;

### Chemical Name: 4-methyl-N-(2-(4-methylpiperazin-1-yl)ethyl)-6-(naphthalen-2-yl)pyridazin-3-amine

| Properties: | Value |
|---|---|
| MW | 361.48 |
| cLogP | 3.17 |
| PSA | 44.29 |
| Rotatable Bonds | 5 |
| HBA (N + O) | 5 |
| HBD (NH + OH) | 1 |

### Synthesis of MW073.

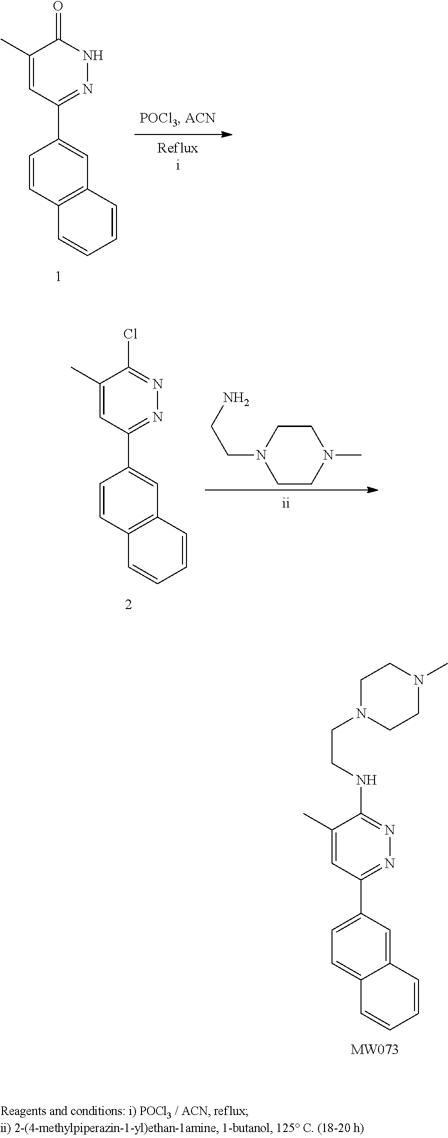

Step (i): 4-methyl-6-(naphthalen-2-yl) pyridazin-3(2H)-one (1), (55.75 gm, 0.24 mol), was treated with phosphorous oxychloride in can at reflux for approximately 4 hours the reaction was found to be complete by LC-MS. The reaction went cleanly to the chloride derivative (ref Wu et. al. 2007). The product was worked up and final recovery was 79.5 grams as a sandy orange solid. The solids were slurred in water and the pH of the mixture was adjusted with ammonium hydroxide to pH 9-10. The treatment was a solid-to-solid conversion. The resulting solids were isolated by vacuum filtration and dried in a vacuum oven to give 51.7 (˜86%) of grams of 2, a light tan solid. Purity by HPLC was 99.9%

Step (ii): 3-chloro-4-methyl-6-(naphthalen-2-yl)pyridazine (2) (35.0 gm, 137 mmol) was charged to a heavy walled, screw-top vessel. To this was added 2-(4-methyl-piperazin-1-yl)-ethylamine (105 gm, 3 eqv, 5.4 mol). The vessel head space was purged with nitrogen, sealed and the heated to 125° C., on magnetic stirrer for 20 h (found to be complete by HPLC). The mixture was cooled to an ambient temperature where a precipitate formed. The solids were removed by vacuum filtration and discarded. The filtrate was diluted with ethyl acetate (200 ml) and the resulting solution was added to a 25 wt. % K₂CO₃ solution (80 gm). The upper, organic layer was isolated, and the lower aqueous phase was back extracted with ethyl acetate (200 ml) where an emulsion formed. The organic layer first isolated (225 g) began to form a crystalline mass. The resulting slurry was heated to reflux which resulted in a solution. Some ethyl acetate was removed by distillation, reducing the product solution to about 155 gm which gave a crystalline solid mass upon cooling. The mixture was refrigerated overnight, and the solids were subsequently broken up and isolated by vacuum filtration. The solids were washed with ice-cold ethyl acetate then allowed to air dry. Approximately 30 grams of dry solids were recovered. The crude MW073 (30 grams) was dissolved in ethyl acetate (90 ml 3 volumes) at reflux and allowed to cool slowly to room temperature overnight. The resulting crystals were isolated by vacuum filtration and the filter cake was rinsed with ice-cold ethyl acetate (30 ml, 1 volume). The solids were air dried then dried under vacuum (50° C., high vacuum, nitrogen bleed) to give the product as a pale-yellow crystalline solid. Mass recovery of MW073 was 16.4 gm and purity TAN).

### Analytical:

HPLC: 98.6% (HPLC)

HPLC (tᵣ/purity): 10.21 min

ESI (m/z): 362.2339 (MH+).

HRMS: HRMS calculated for C₂₂H₂₇N₅ 316.22665, found 316.22662

NMR: ¹H NMR (CDCl₃): δ 8.38 (s, 1H), 8.25-8.23 (dd, J=6.0 & 12 Hz, 2H), 7.93-7.90 (m, 2H), 7.87-7.85 (m, 1H), 7.60 (s, 1H), 7.51-7.47 (m, 2H), 4.73 (s, 1H), 3.75-3.74 (t, 2H), 2.76-2.74 (t, 2H), 2.57 (br, m, 7H), 2.31 (s, 3H), 2.24 (s, 3H)

### Synthesis of MW109 (4-methyl-N-(2-(4-methylpiperazin-1-yl)ethyl)-6-phenylpyridazin-3-amine)

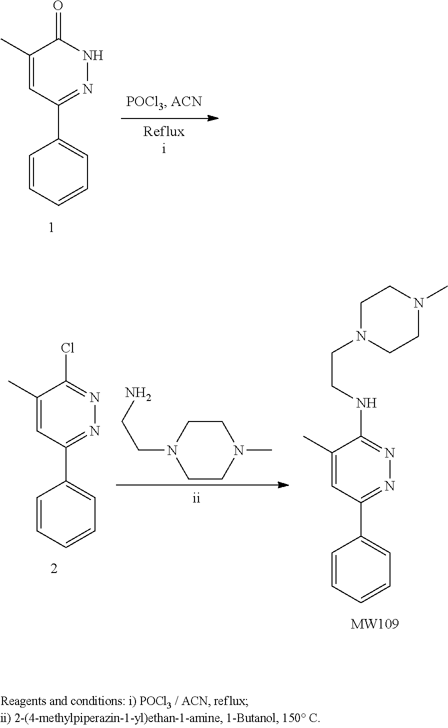

Step (i): 4-methyl-6-phenylpyridazin-3(2H)-one (1), (22.2 g, 120 mmol) was treated with phosphorous oxychloride can ACN at reflux for approximately 2.5 hours the reaction was found to be complete by LC-MS. The reaction went cleanly to the chloride derivative (ref Wu et. al. 2007). The product was worked up and final recovery was 20 grams as light pink powder, 3-chloro-4-methyl-6-phenylpyridazine (2) (˜90%).

¹H NMR (DMSO-d6): δ 8.32 (s, 1H), 8.13 (d, J=6.0 Hz, 2H), 7.57-7.56 (m, 3H), 2.45 (s, 3H), HPLC (tr/purity): 17.07 min, >95%. ESI m/z (MeOH) 205.49 (MH+).

Step (ii): 3-chloro-4-methyl-6-phenylpyridazine (2), (5 g, 24.4 mmol) was suspended in 75 ml 1-butanol and was added 2-(4-methylpiperazin-1-yl)ethan-1-amine (21 g, 146 mmol) in a heavy wall reaction vial. Blanket the reaction mixture with Ar and sealed the vial with the screwcap and heated at 1500 C with stirring on for ˜72 h. Reaction was monitored by HPLC. The reaction mixture cooled to ambient temperature, deionized water added, and the mixture subjected to repeat extraction with dichloromethane. The organic layers were taken to drying with anhydrous sodium sulfate and concentration in vacuo. The final products were purified by silica gel column chromatography using ethyl acetate:hexane (80:20) for elution and final processing product appeared as white powder, crystallization produced 3.4 g as white crystals, yield >50% and purity 98% (HPLC-TAN).

### Analytical:

HPLC (tr/purity): 9.28 min, >98%.

ESI (m/z): 312.21 (M+1).

¹H NMR (500 MHz, MeOD): δ 7.88 (dd, J=1.5, 6.55 Hz, 2H), 7.62 (dd, 1.2, 1.25 Hz, 1H), 7.47-7.37 (m, 3H), 3.74-3.71 (m, 2H), 3.29 (s, br, 1H), 2.76-2.73 (m, 8H), 2.54 (m, 1H); 2.29 (d, J=1.85 Hz, 3H), 2.24 (d, J=1.45 Hz, 3H).

HRMS (m/z): (M+1) calculated for C₁₈H₂₆N₅ is 312.21882, found 312.2183.3H.

MW073 was tested for activity in various cell-based agonist and antagonist screens as shown in Table II.

TABLE II
MW073 Tested for Activity in 164 Cell-
Based Agonist and Antagonist Screens

| GPCR | GPCR | GPCR | GPCR |
|---|---|---|---|
| A1 | CXCR6 | LPA1 | PK2 |
| A2A | CCK1 (CCKA) | LPA2 | DP1 |
| A2B | CCK2 (CCKB) | LPA3 | EP1 |
| A3 | C3aR | S1P2 | EP2 |
| α1A | ChemR23 | S1P3 | EP3 |
| α1B | CRF1 | S1P4 | EP4 |
| α1D | CRF2α | S1P5 | FP |
| α2A | XCR1/GPR5 | MCH1 | IP (PGI2) |
| α2B | D1 | MCH2 | TP (TXA2/PGH2) |
| α2C | D2L | MC1 | PAR1 |
| β1 | D3 | MC2 | PAR2 |
| β2 | D4.4 | MC3 | P2Y1 |
| β3 | D5 | MC4 | P2Y2 |
| AT1 | ETA | MC5 | P2Y4 |
| APJ | ETB | MT2 (ML1B) | P2Y6 |
| TGR5 | FFA1 | motilin | P2Y11 |
| BB1 | FFA2 | M1 | PTH2 |
| BB2 | FFA3 | M2 | RXFP1 |
| BB3 | FFA4 (GPR120) | M3 | 5-HT1A |
| B1 | GnRH | M4 | 5HT1B |
| B2 | GPR39 | M5 | 5-HT2A |
| CGRP | OXGR1 GPR99 | MrgD | 5-HT2B* |
| CT | GPR103/QRFP | MRGX1/ | 5-HT2C |
| | | MRGPRX1 | |
| CaS | GPR109A | MRGX2/ | 5-HT4e |
| | | MRGPRX2 | |
| CB1 | GPR119 | NPS | 5-HT6 |
| CB2 | FPR1 | NPBW1 | 5-HT7 |
| CCR2 | GABAB | NK1 | sst1 |
| | (B1b/B2) | | |
| CCR3 | GAL1 | NK2 | sst2 |
| CCR4 | GAL2 | NK3 | sst3 |
| CCR5 | GIP | NMU1 | sst4 |
| CCR6 | GLP-1 | NMU2 | sst5 |
| CCR7 | secretin | NTS1 (NT1) | SUCNR1/GPR91 |
| CCR8 | GHRH | δ (DOP) | TRH1 |
| CCR9 | Ghrelin/GHSR-1a | κ (KOP) | UT |
| CCR10 | H1 | μ (MOP) | PAC1 (PACAP) |
| CX3CR1 | H2 | NOP | VPAC1 (VIP1) |
| CXCR1 | H3 | OX1 | Y4 |
| CXCR2 | KISS1/GPR54 | OX2 | OT |
| CXCR3 | BLT1 (LTB4) | PAF | V1a |
| CXCR4 | CysLT1 (LTD4) | PTH1 | V1B |
| CXCR5 | CysLT2 (LTC4) | PK1 | V2 |

*Positive for 5-HT2B antagonist activity and negative for 5-HT2B agonist activity; Negative for both agonist and antagonist activities for all other GCPRs.

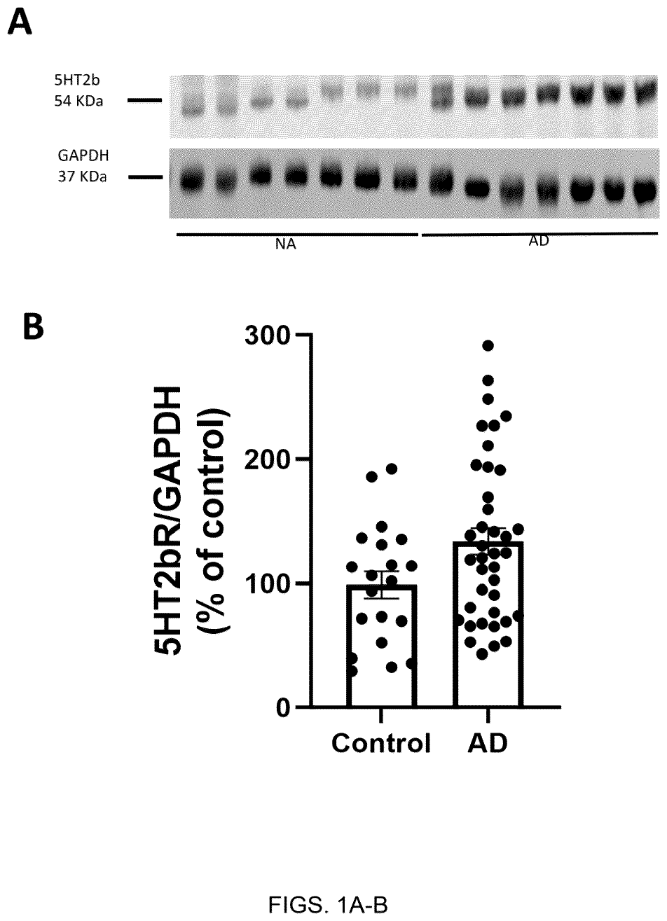

FIG. 1A-B illustrate that 5HT2b receptor expression is increased in the cortex from post-mortem specimens from AD patients (39 sporadic AD: 22 females and 17 males) and age-matched non-demented controls (20 individuals: 7 females and 13 males). Post-mortem interval was recorded. Individuals were characterized for their Braak and Braak stage (VI-0), and CERAD score (C-0). AD patients had no other diseases that might have contributed to the clinical deficits. A) Representative western blotting of 5HT2bR level in human brain samples. B) Cumulative graphs (t-test p=0.039).

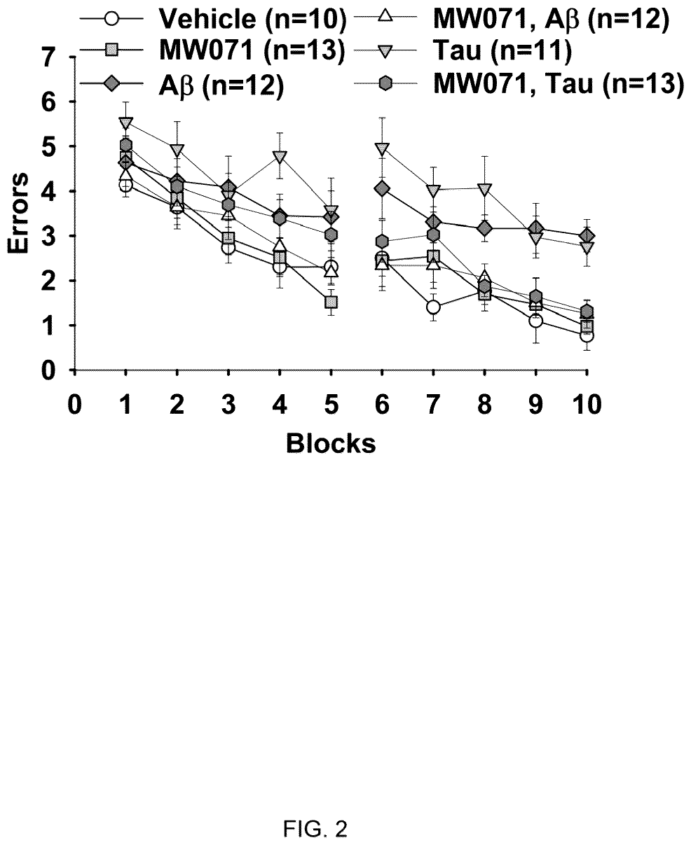

FIG. 2 shows efficacy of MW071 in Alzheimer relevant mouse models characterized by Aβ and tau oligomer elevation through their exogenous application using a test of spatial memory, the radial arm water maze (RAWM). MW071 (5 mg/Kg, i.p., 5 mg/Kg, i.p., 1 injection 30 min before the first trial and the seventh trial both on day 1 and 2) protects C57Bl6 mice against the impairment of spatial memory by infusion of 200 nM Aβ- or 500 nM tau into dorsal hippocampi bilaterally, while MW071 alone does not affect performance in mice infused with vehicle. RAWM: ANOVA for repeated measures among all groups (day 2): F(5,64)=6.130, p=0.0001. One-way ANOVA for block 10: F(5,65)=8.552, p<0.0001; Bonferroni's p<0.0001 vehicle vs. Aβ; p=0.0006 vehicle vs. Tau; p=0.0013 Aβ vs. Aβ+MW071; p=0.108 Tau vs. Tau+MW071 and p=1 vehicle vs. MW071. The number of animals is indicated on the graph.

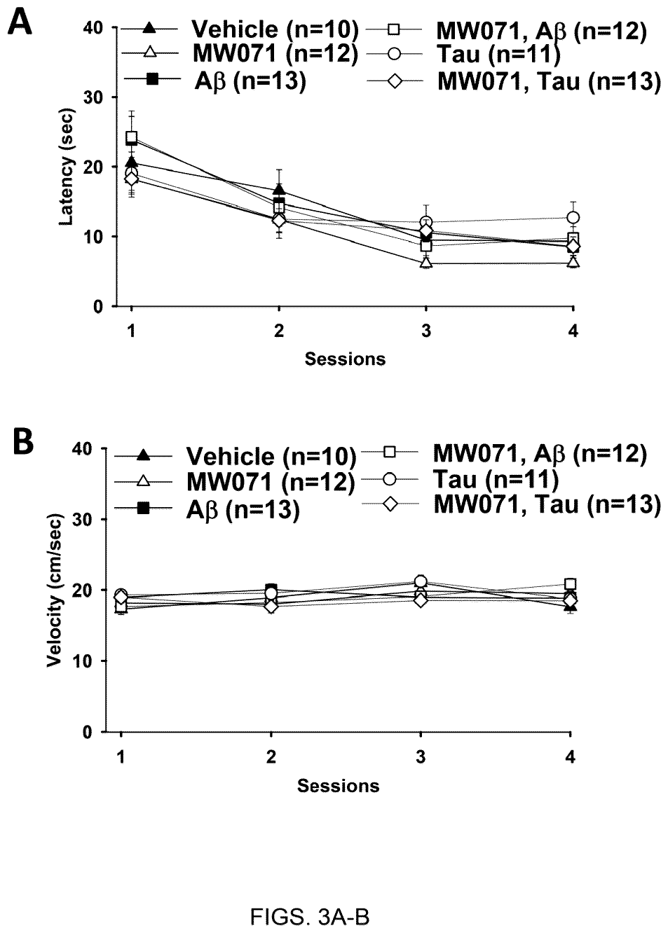

FIG. 3A-B illustrate that MW071 has no effect on vision, motility, or motivation in C57Bl6 mice infused with Tau or Aβ oligomers. Testing with the visible platform task does not reveal any difference in (A) time to reach the visible platform (2-way ANOVA: F(5,65)=0.4625, p=0.8027) and (B) average speed (2-way ANOVA: F(5,65)=0.6686, p=0.6487) after administration of MW071 (5 mg/Kg, i.p., 30 min prior the 1st session over 2 days). The number of animals is indicated on the graph.

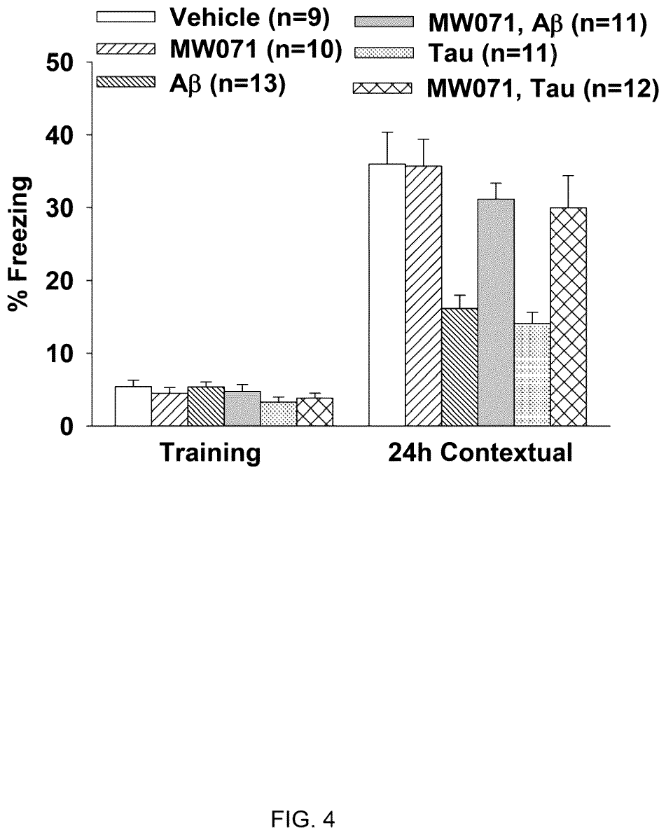

FIG. 4 shows efficacy of MW071 in AD relevant mouse models characterized by Aβ and tau oligomer elevation using a test of associative memory, the contextual fear conditioning (FC). MW071 (5 mg/Kg, i.p., 1 injection 30 min prior to the electric shock) protects C57Bl6 mice against the impairment of associative memory by infusion of 200 nM Aβ- or 500 nM tau into dorsal hippocampi bilaterally, while MW071 alone does not affect memory in vehicle infused mice. FC: One-way ANOVA 24 hr contextual F(5,60)=9.391, p<0.0001; Bonferroni p=0.0002 vehicle vs. Aβ; p<0.0001 vehicle vs. Tau; p=0.0043 Aβ vs. Aβ+MW071; p=0.028 Tau vs. Tau+MW071; p=1 vehicle vs. MW071. The number of animals is indicated on the graph.

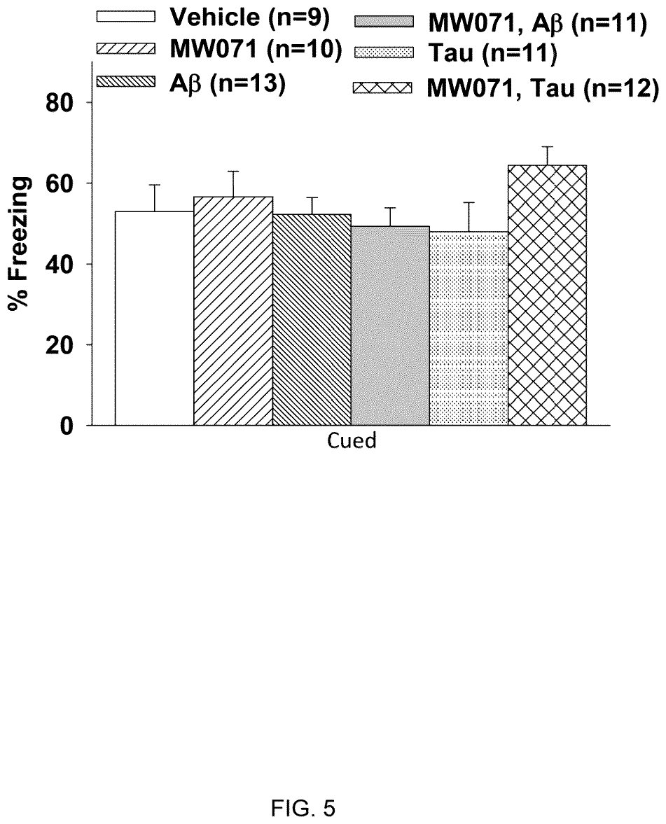

FIG. 5 shows that MW071 has no effect on cued amygdala dependent memory in C57Bl6 mice infused with Tau or Aβ oligomers. Freezing responses during the auditory cued conditioning are not significantly different among the groups (one-way ANOVA: F(5,60)=1.203, p=0.3189) after administration of MW071 (5 mg/Kg, i.p., 1 injection 30 min prior to the sound). The number of animals “n” is indicated on the graph.

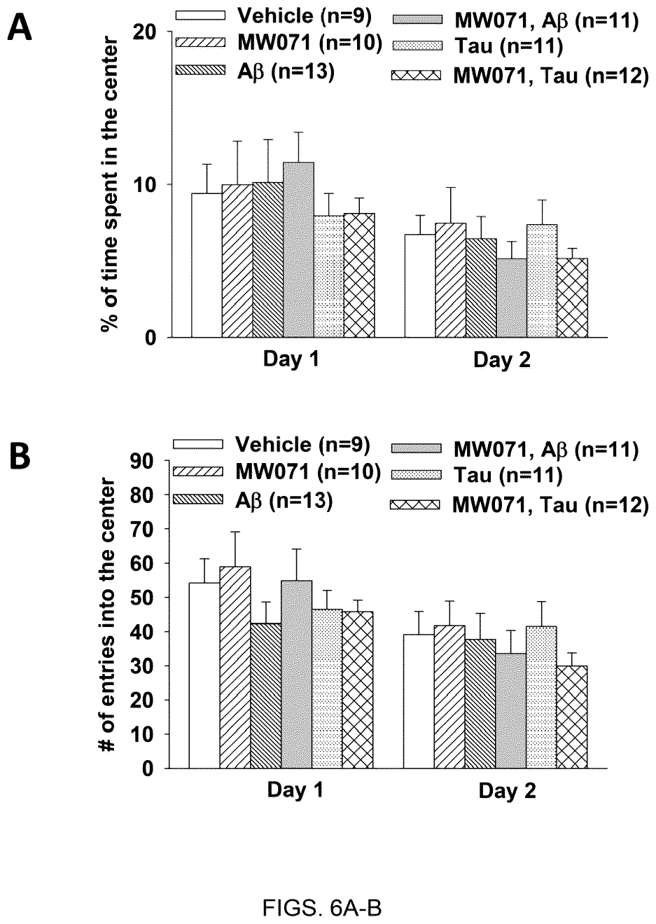

FIG. 6A-B illustrate that MW071 has no effect on exploratory behavior in C57Bl6 mice infused with Tau or Aβ oligomers. Open field test shows a similar percentage of time spent in the center (A) (one-way ANOVA day 1: F(5,60)=0.3921, p=0.8524; day 2: F(5, 60)=0.4895, p=0.7828) and (B) number of entries into the center among all conditions (one-way ANOVA day 1: F(5, 60)=0.8351, p=0.53; day 2: F(5,60)=0.5069, p=0.7699), indicating no differences in exploratory behavior. MW071 was administered at a concentration of 5 mg/Kg (i.p., 1 injection 30 min prior to the test, both on day 1 and 2). The number of animals “n” is indicated on the graph.

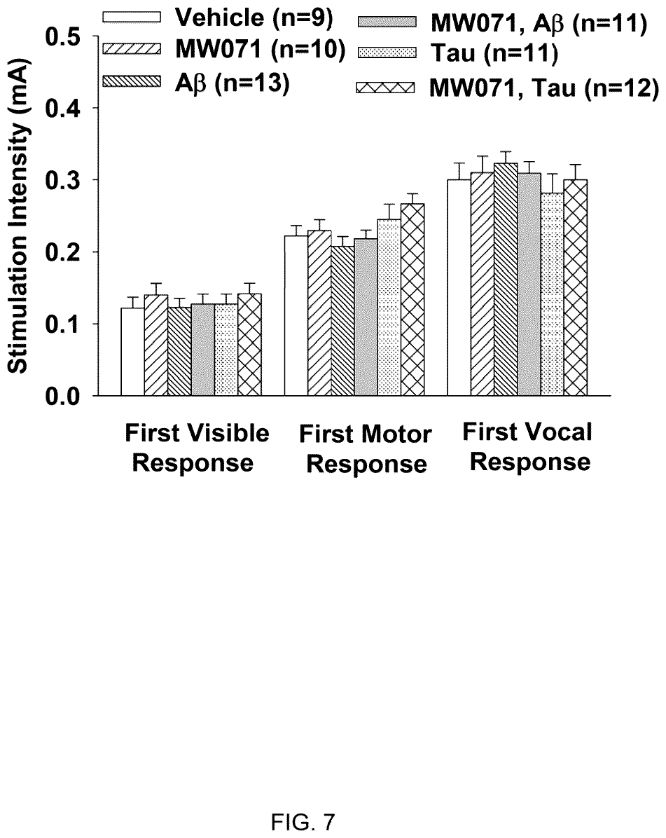

FIG. 7 shows that MW071 has no effect on animal capability of perceiving the electric shock in C57Bl6 mice infused with Tau or Aβ oligomers. No difference is detected among the groups during assessment of the sensory threshold after administration of MW071 (5 mg/Kg, i.p., 1 injection 30 min prior to the test). One-way ANOVA among all: for visible response F(5,60)=0.3507, p=0.8798; for motor response F(5,60)=2.083, p=0.0800 and for audible response F(5, 60)=0.4479, p=0.8132. The number of animals “n” is indicated on the graph.

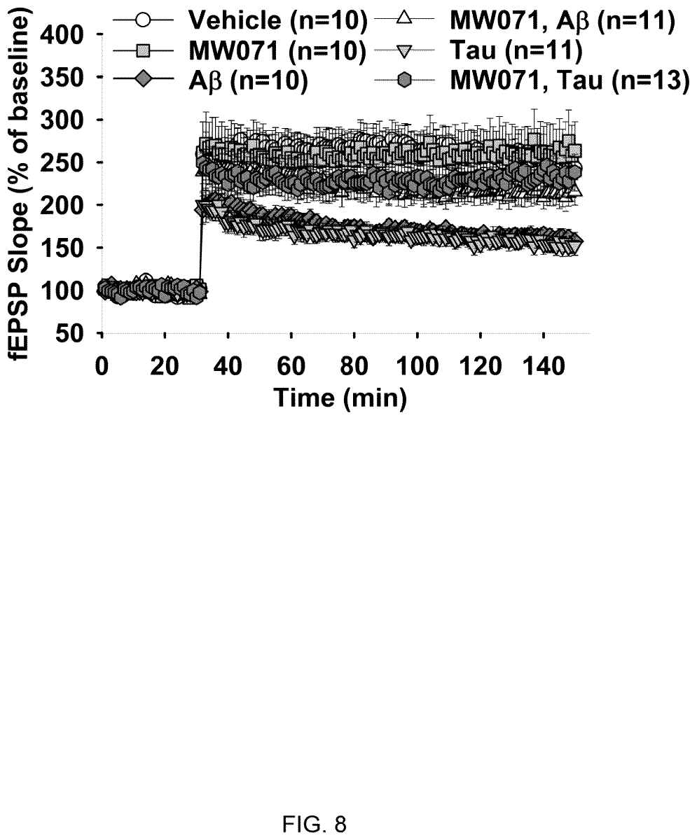

FIG. 8 shows efficacy of MW071 in AD relevant mouse models characterized by Aβ and tau oligomer elevation using long-term potentiation (LTP), as a test of synaptic plasticity. Perfusion with MW071 (10 μM) rescues LTP defect in hippocampal slices from C57Bl6 mice treated with 200 nM Aβ- or 50 nM tau-oligomers. MW071 alone does not affect potentiation. ANOVA for repeated measures between groups: F(1,19)=27.85, p<0.0001 vehicle vs. Tau; F(1,15)=22.48, p=0.0003 vehicle vs. Aβ; F(1,22)=10.38, p=0.0039 Tau vs. Tau+MW071; F(1,16)=7.20, p=0.0163 Aβ vs. Aβ+MW071; (F(1, 18)=0.0082, p=0.92 vehicle vs. MW071. Vehicle: The number of slices “n” is indicated on the graph. N=10 (5 males, 4 females), MW071: N=10 (4 males, 3 females), Aβ: N=10 (4 males, 3 females), Aβ+MW071: N=11 (4 males, 4 females), Tau: N=11 (5 males, 5 females), Tau+MW071: N=13 (6 males, 6 females).

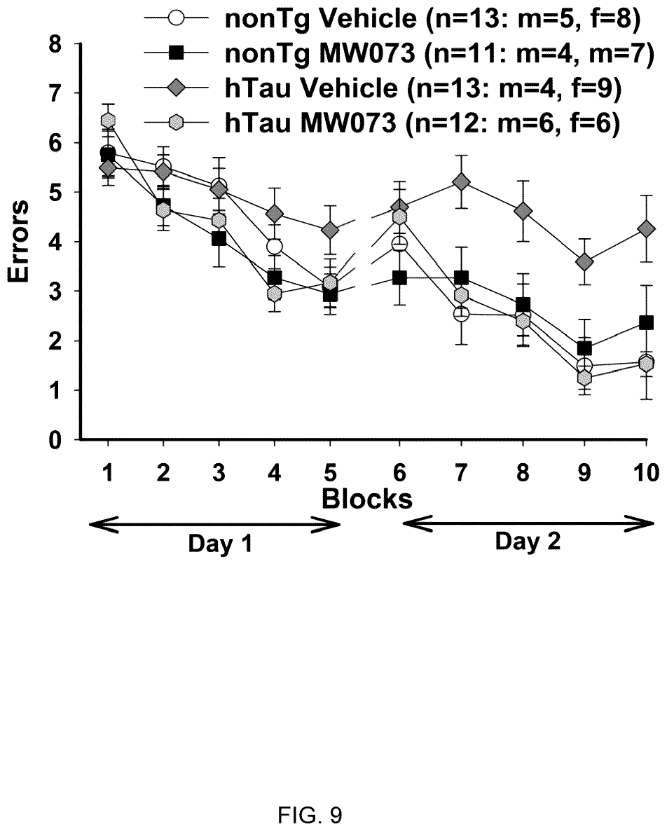

FIG. 9 shows the efficacy of MW073 following intervention at a disease state onto the hTau/Mapt-KO mouse model of tau elevation using a test of spatial memory, the RAWM. MW073 (5 mg/Kg, o.s., daily from the age of 11 months for 45 days) protects hTau/Mapt-KO mice against the impairment of spatial memory, while MW073 alone in control non-transgenic littermates does not affect performance in non-transgenic (nonTg) littermates. RAWM: 2 way-ANOVA for repeated measures among all groups (day 2): F(3,45)=4.732, p=0.0059. One-way ANOVA for block 10: F(3,45)=4.862, p=0.0052; One-way ANOVA for block 9: F(3,45)=5.728, p=0.0021 One-way ANOVA for block 8: F(3,45)=3.179, p=0.0329 One-way ANOVA for block 7: F(3,45)=4.624, p=0.0067 One-way ANOVA for block 6: F(3,45)=0.9262, p=0.4359. The number of animals “n” is indicated on the graph.

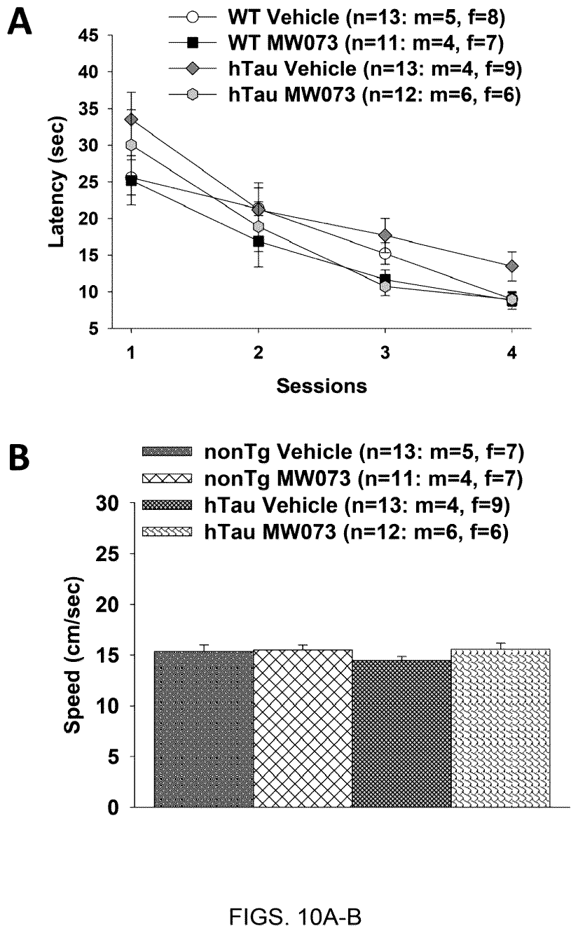

FIG. 10A-B shows that MW073 has no effect on vision, motility, or motivation following intervention at a disease state on the hTau/Mapt-KO mouse model of Alzheimer's disease. Testing with the visible platform task does not reveal any difference in time to reach the visible platform (2-way ANOVA: F(3,45)=1.500, p=0.2273) (A) and average speed (1-way ANOVA: F(3,45)=0.9082, p=0.4446) (B). MW073 was administered at a concentration of 5 mg/Kg (o.s., daily, from the age of 11 months for 45 days). The number of animals “n” is indicated on the graph.

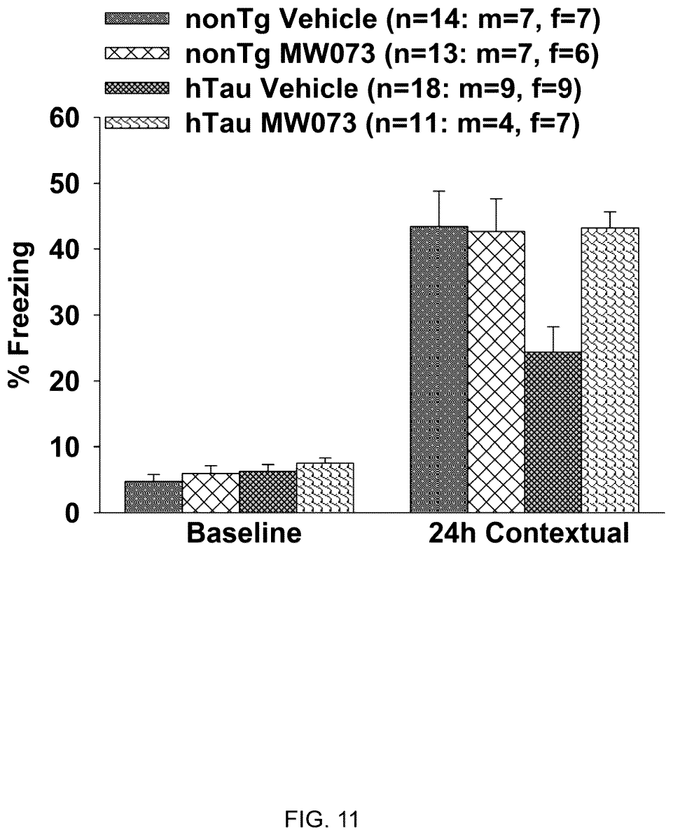

FIG. 11 show the efficacy of MW073 following intervention at a disease state onto the hTau/Mapt-KO mouse model of tau elevation using a test of associative memory, the contextual fear conditioning (FC). MW073 (5 mg/Kg, o.s., daily, from the age of 11 months for 150 days) protects hTau/Mapt-KO mice against the impairment of associative memory, while MW073 alone in control non-transgenic littermates does not affect memory. FC: ANOVA F(3,52)=8.834, p=0.0001; Bonferroni p=0.0003 nonTg vehicle vs. hTau vehicle, p=0.0009 hTau vehicle vs. hTau MW073, p>0.999 nonTg vehicle vs. nonTg MW073. The number of animals “n” is indicated on the graph.

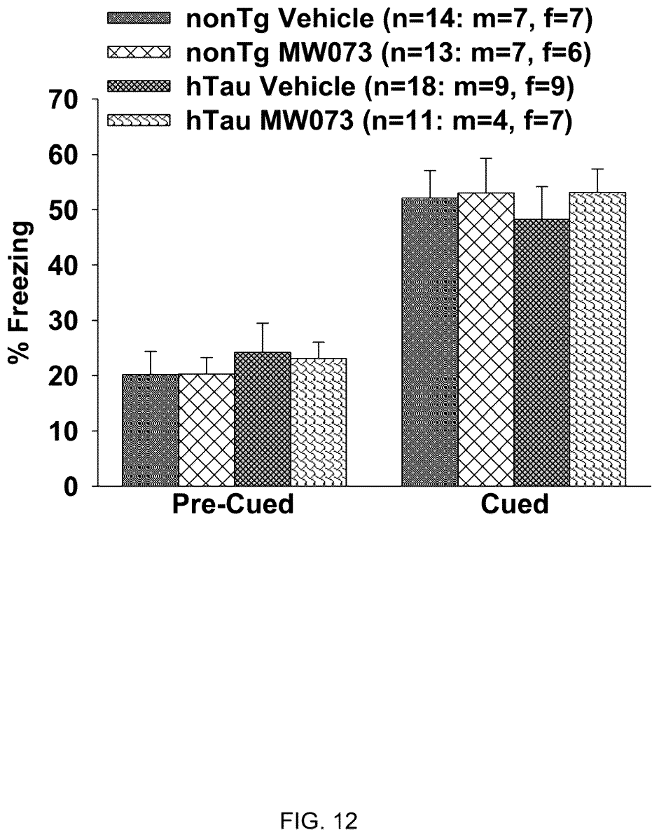

FIG. 12 show that MW073 has no effect on cued amygdala dependent memory following intervention at a disease state onto the hTau/Mapt-KO mouse model of tau elevation. Freezing responses during the auditory cued conditioning are not significantly different among the groups (1-way ANOVA: F(3,52)=0.3199, p=0.8110). MW073 was administered at a concentration of 5 mg/Kg (o.s., daily, from the age of 11 months for 150 days). The number of animals “n” is indicated on the graph.

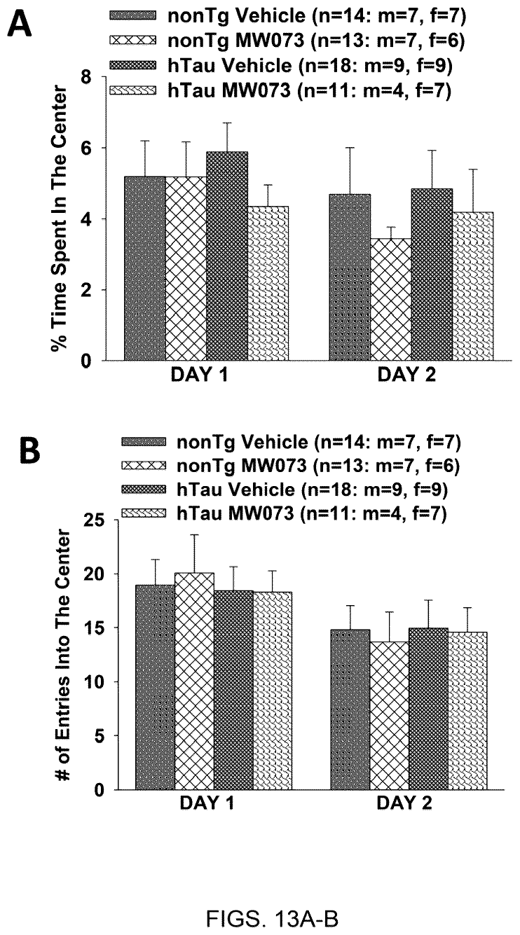

FIG. 13A-B shows that MW073 has no effect on exploratory behavior following intervention at a disease state onto the hTau/Mapt-KO mouse model of tau elevation. Open field test shows a similar percentage of time spent in the center (day 1: F(3,52)=0.5200, p=0.5274; day 2: F(3, 52)=0.4101, p=0.7464) (A) and number of entries into the center among all conditions (day 1: F(3, 52)=0.07868, p=0.9713; day 2: F(3,52)=0.03897, p=0.9896) (B), indicating no differences in exploratory behavior. MW073 was administered at a concentration of 5 mg/Kg (o.s., daily, from the age of 11 months for 150 days). The number of animals “n” is indicated on the graph.

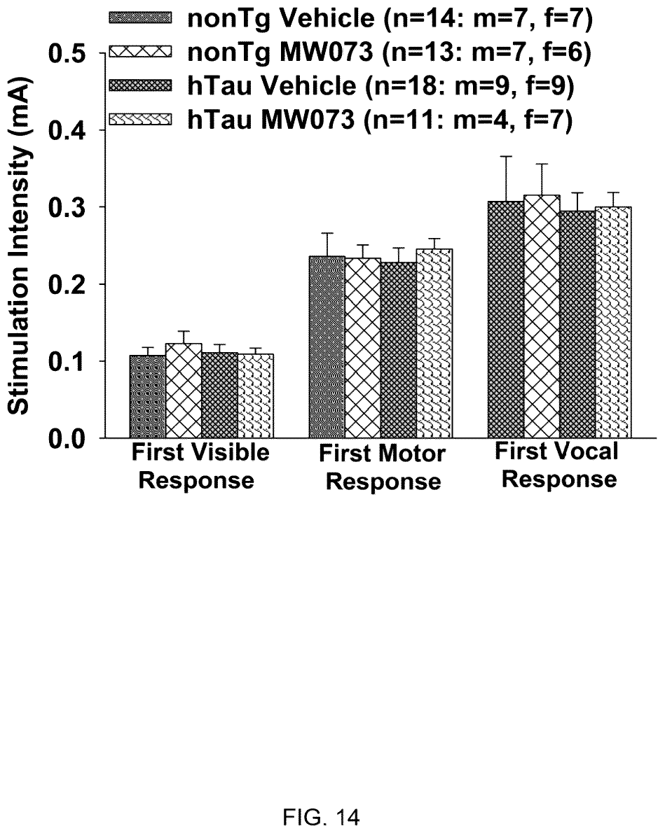

FIG. 14 shows that MW073 has no effect on animal capability of perceiving the electric shock following intervention at a disease state onto the hTau/Mapt-KO mouse model of tau elevation. No difference is detected among the groups during assessment of the sensory threshold. MW073 was administered at a concentration of 5 mg/Kg (o.s., daily, from the age of 11 months for 150 days). One-way ANOVA among all: for visible response F(3,52)=0.5907, p=0.6238; for motor response F(3,52)=1.032, p=0.3863 and for audible response F(3, 52)=0.1094, p=0.9542. The number of animals “n” is indicated on the graph.

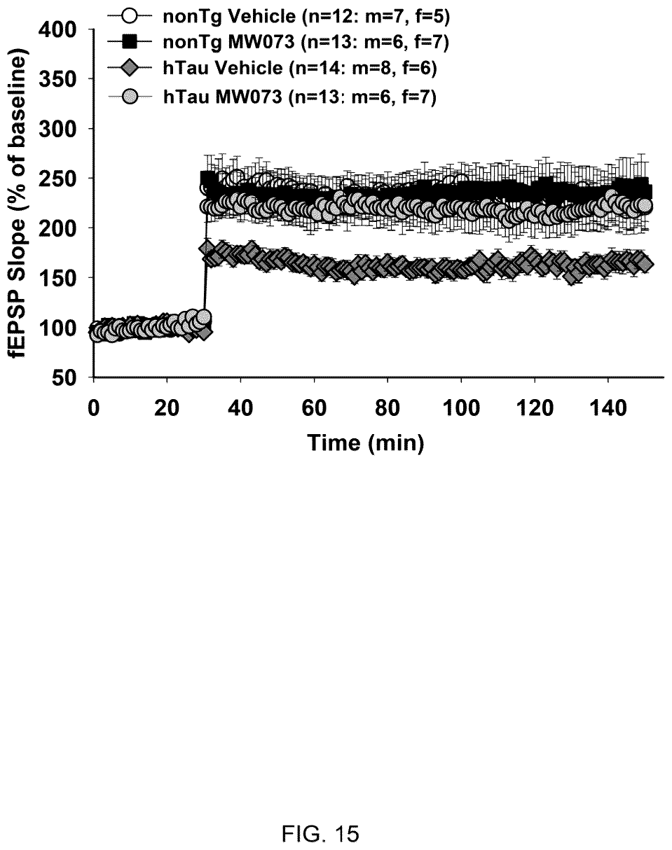

FIG. 15 shows the efficacy of MW073 in the hTau/Mapt-KO mouse model of tau elevation following intervention at a disease state using LTP as a test of synaptic plasticity. MW073 (5 mg/Kg, o.s., daily, from the age of 11 months for 160 days) restores animal capability of undergoing potentiation in the hTau-Mapt-KO mouse model. MW073 alone in control non-transgenic littermates does not affect potentiation. 2-way ANOVA F(3,48)=3.794, p=0.0160 ANOVA for repeated measures between groups: F(1,23)=1.093e-005, p=0.9974 nonTg vehicle vs nonTg+MW073. F(1,24)=14.97, p=0.0007 nonTg vehicle vs. hTau vehicle; F(1,25)=8.425, p=0.0076 hTau vehicle vs. hTau+MW073; F(1,26)=0.2153, p=0.6469 nonTg+MW073 vs. hTau+MW073; The number of slices “n” is indicated on the graph.

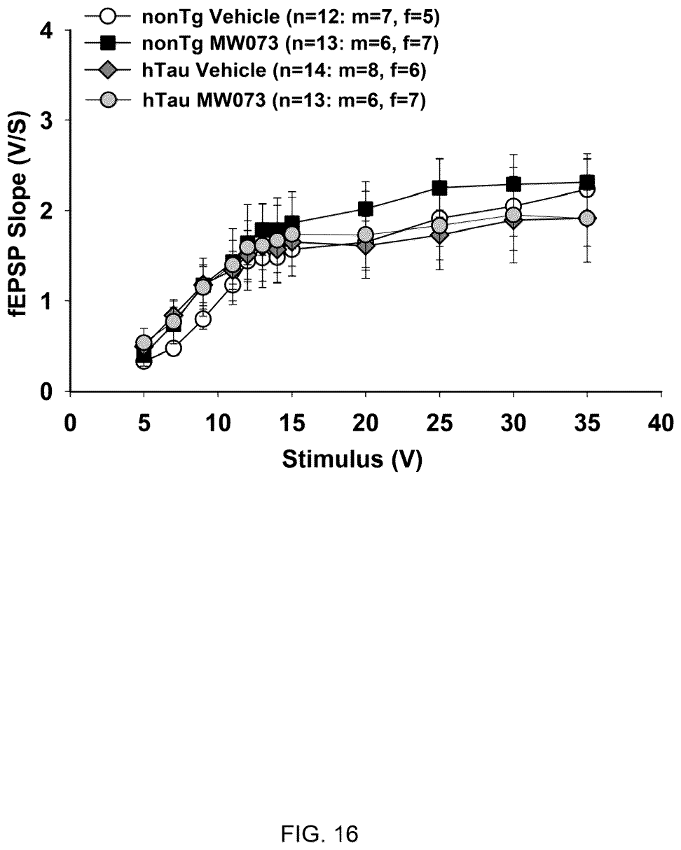

FIG. 16 shows that MW073 has no effect on basal neurotransmission in the hTau/Mapt-KO mouse model of tau elevation following intervention at a disease state. MW073 (5 mg/Kg, o.s., daily, from the age of 11 months for 160 days) does not alter the input/output relationship in a test of basal synaptic transmission. 2-Way-ANOVA: F(3,48)=0.1492, p=0.9297. The number of slices “n” is indicated on the graph.

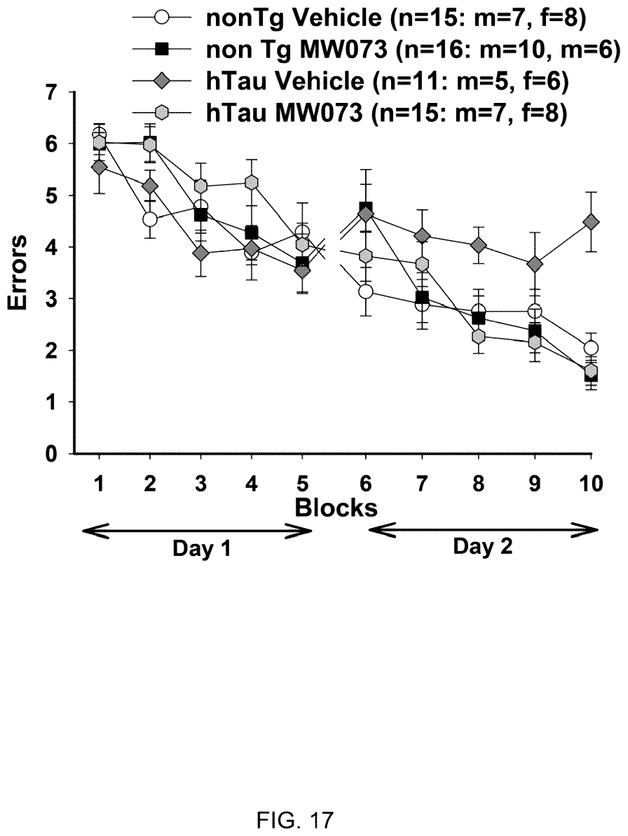

FIG. 17 shows the efficacy of MW073 in a prevention trial onto the hTau/Mapt-KO mouse model of tau elevation using a test of spatial memory, the RAWM. MW073 (5 mg/Kg, o.s., daily, from the age of 7 months for 180 days) protects hTau/Mapt-KO mice against the impairment of spatial memory, while MW073 alone in non-transgenic control littermates does not affect performance. RAWM: 2 way-ANOVA for repeated measures among all (day 2): F(3,53)=3.537, p=0.0207. One-way ANOVA for block 10: F(3,53)=13.35, p<0.0001; One-way ANOVA for block 9: F(3,53)=1.458, p=0.2365 One-way ANOVA for block 8: F(3,53)=2.666, p=0.0571 One-way ANOVA for block 7: F(3,53)=1.450, p=0.2386 One-way ANOVA for block 6: F(3,53)=1.620, p=0.1956. The number of animals “n” is indicated on the graph.

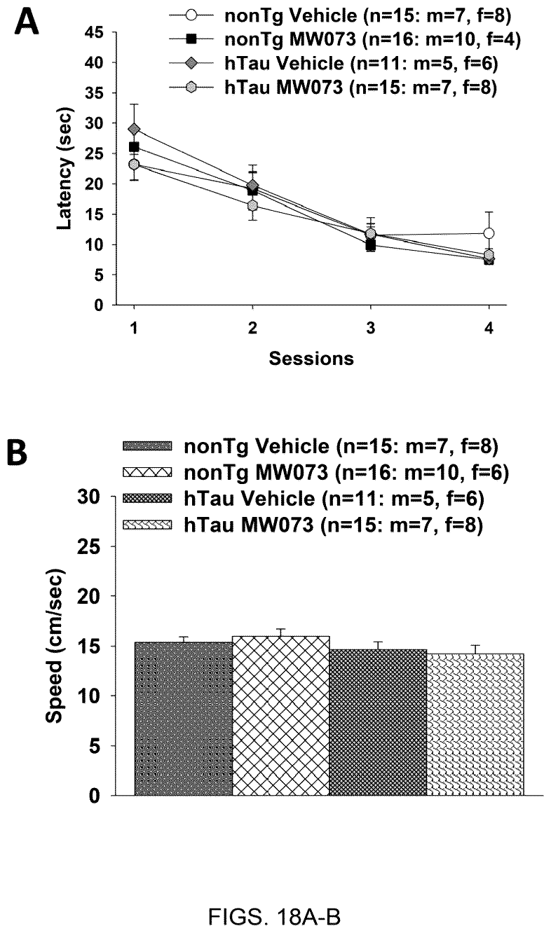

FIG. 18A-B show that MW073 has no effect on vision, motility, or motivation during a prevention trial on the hTau/Mapt-KO mouse model of Alzheimer's disease. Testing with the visible platform task does not reveal any difference in time to reach the visible platform (2-way ANOVA: F(3,53)=0.3723, p=0.7733) (Panel A) and average speed (1-way ANOVA: F(3,53)=1.212, p=0.3144) (Panel B). MW073 was administered at a concentration of 5 mg/Kg (o.s., daily, from the age of 7 months for 180 days). The number of animals “n” is indicated on the graph.

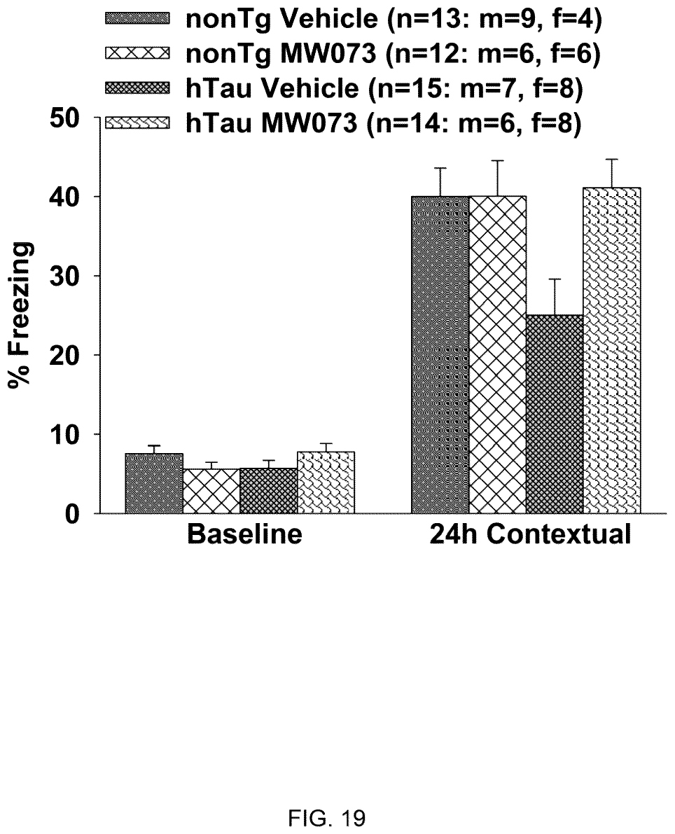

FIG. 19 shows the efficacy of MW073 in a prevention trial onto the hTau/Mapt-KO mouse model of tau elevation using a test of associative memory, the contextual fear conditioning (FC). MW073 (5 mg/Kg, o.s., daily, from the age of 7 months for 240 days) protects hTau/Mapt-KO mice against the impairment of associative memory, while MW073 alone in non-transgenic control littermates does not affect memory. FC: ANOVA F(3,50)=3.432, p=0.0238; Bonferroni p=0.00465 nonTg vehicle vs. hTau vehicle, p=0.00258 hTau vehicle vs. hTau MW073, p>0.999 nonTg vehicle vs. nonTg MW073. The number of animals “n” is indicated on the graph.

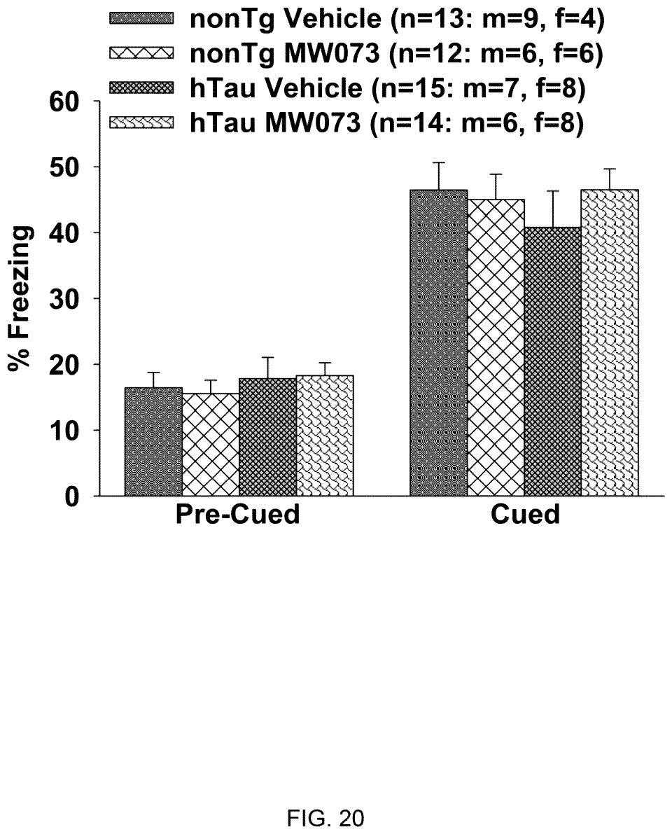

FIG. 20 shows that MW073 has no effect on cued amygdala dependent memory in a prevention trial onto the hTau/Mapt-KO mouse model of tau elevation. Freezing responses during the auditory cued conditioning are not significantly different among the groups (1-way ANOVA: F(3,50)=0.2502, p=0.8608). MW073 was administered at a concentration of 5 mg/Kg (o.s., daily, from the age of 7 months for 240 days). The number of animals “n” is indicated on the graph.

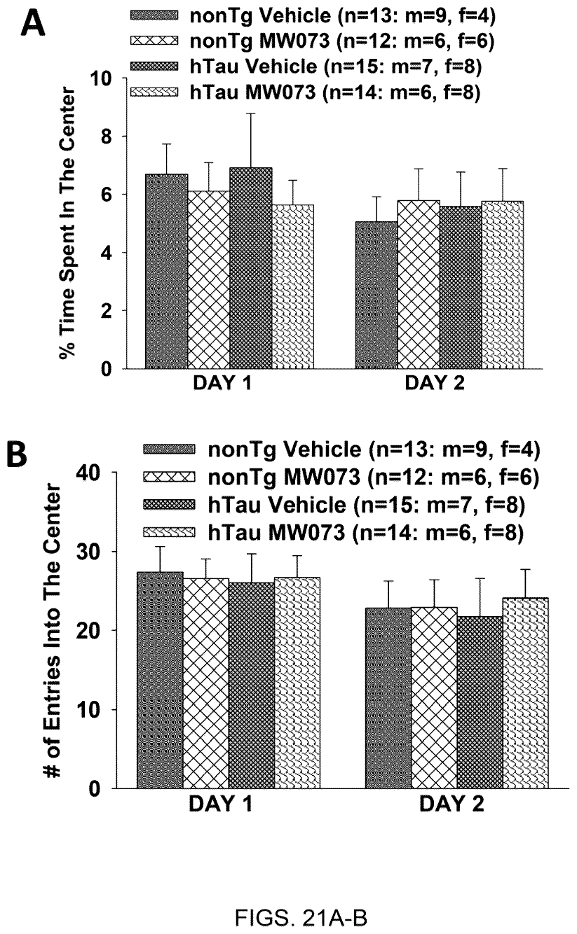

FIG. 21A-B shows that MW073 has no effect on exploratory behavior in a prevention trial onto the hTau/Mapt-KO mouse model of tau elevation. Open field test shows a similar percentage of time spent in the center (day 1: F(3,50)=0.2026, p=0.8941; day 2: F(3, 50)=0.08514, p=0.9678) (A) and number of entries into the center among all conditions (day 1: F(3, 50)=0.02985, p=0.9930; day 2: F(3,50)=0.05, p=0.9818) (B), indicating no differences in exploratory behavior. MW073 was administered at a concentration of 5 mg/Kg (o.s., daily, from the age of 7 months for 240 days). The number of animals “n” is indicated on the graph.

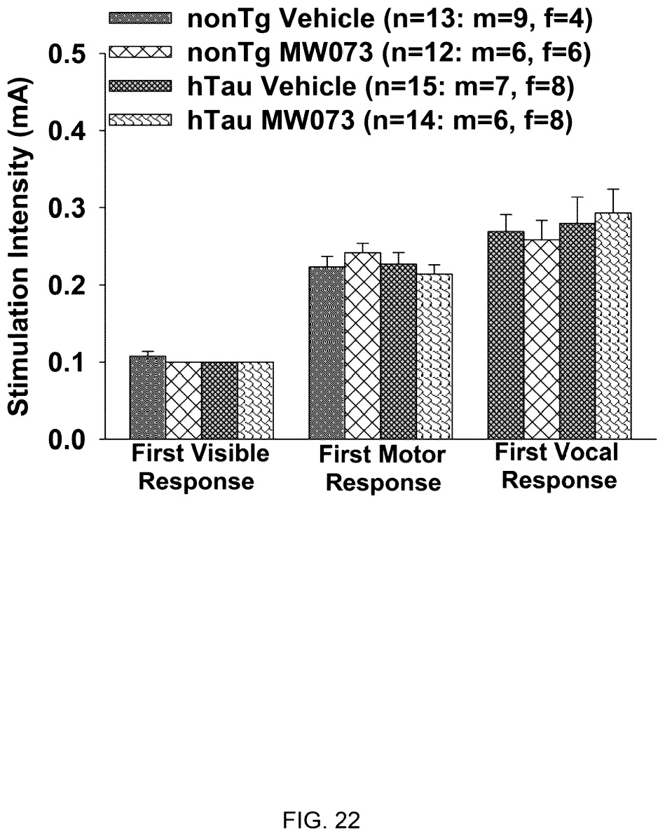

FIG. 22 shows that MW073 has no effect on animal capability of perceiving the electric shock in a prevention trial onto the hTau/Mapt-KO mouse model of tau elevation. No difference is detected among the groups during assessment of the sensory threshold. One-way ANOVA among all: for visible response F(3,50)=1.055, p=0.3768; for motor response F(3,50)=0.6997, p=0.5567 and for audible response F(3, 50)=0.2796, p=0.8398. MW073 was administered at a concentration of 5 mg/Kg (o.s., daily, from the age of 7 months for 240 days). The number of animals “n” is indicated on the graph.

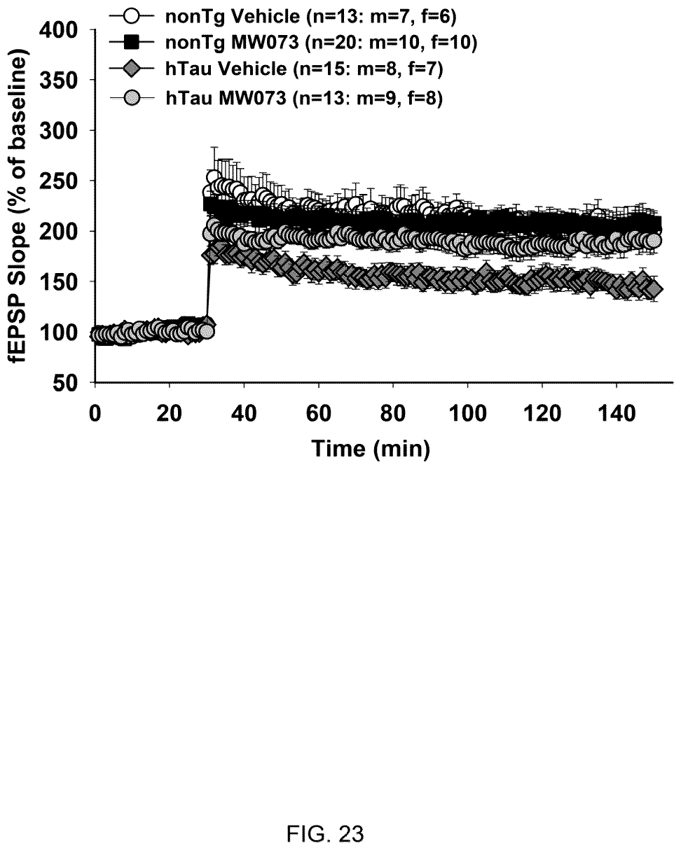

FIG. 23 shows the efficacy of MW073 in the hTau/Mapt-KO mouse model of tau elevation in a prevention trial using LTP as a test of synaptic plasticity. MW073 (5 mg/Kg, o.s., daily, from the age of 7 months for 240 days) prevents the LTP defect in the hTau-Mapt-KO mouse model of Alzheimer's Disease. MW073 alone in non-transgenic control littermates does not affect potentiation. 2-way ANOVA F(3,61)=5.305, p=0.0026 ANOVA for repeated measures between groups: F(1,31)=0.1241, p=0.7270 nonTg vehicle vs nonTg+MW073. F(1,26)=11.67, p=0.0021 nonTg vehicle vs. hTau vehicle; F(1,30)=7.238, p=0.011 hTau vehicle vs. hTau+MW073; F(1,35)=1.642, p=0.2085 nonTg+MW073 vs. hTau+MW073. The number of slices “n” is indicated on the graph.

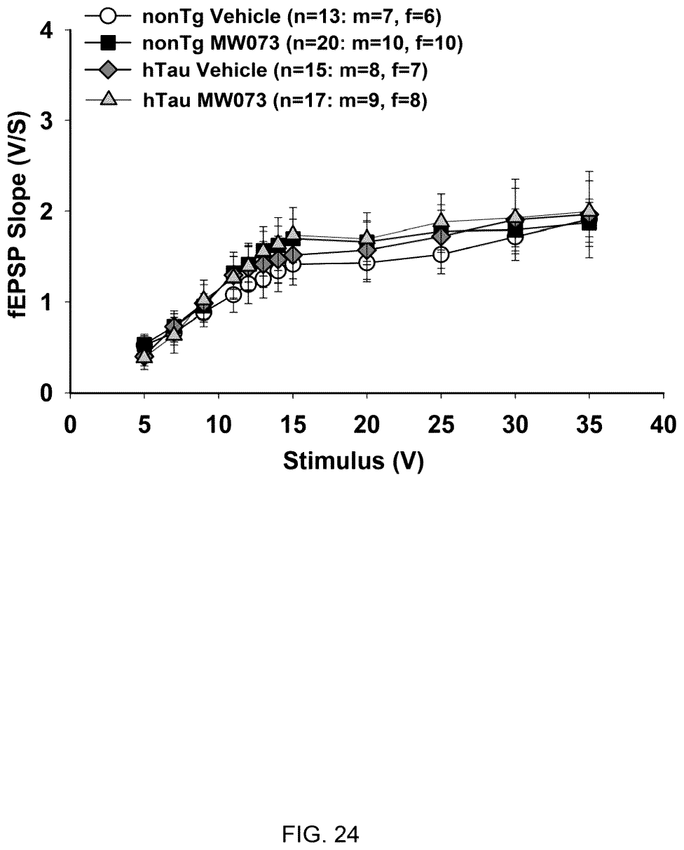

FIG. 24 shows that MW073 has no effect on basal neurotransmission in the hTau/Mapt-KO mouse model of tau elevation during a prevention trial. MW073 (5 mg/Kg, o.s., daily, from the age of 7 months for 240 days) does not alter the input/output relationship in a test of basal synaptic transmission. 2-Way-ANOVA: F(3,61)=0.1294, p=0.9423. The number of slices “n” is indicated on the graph.

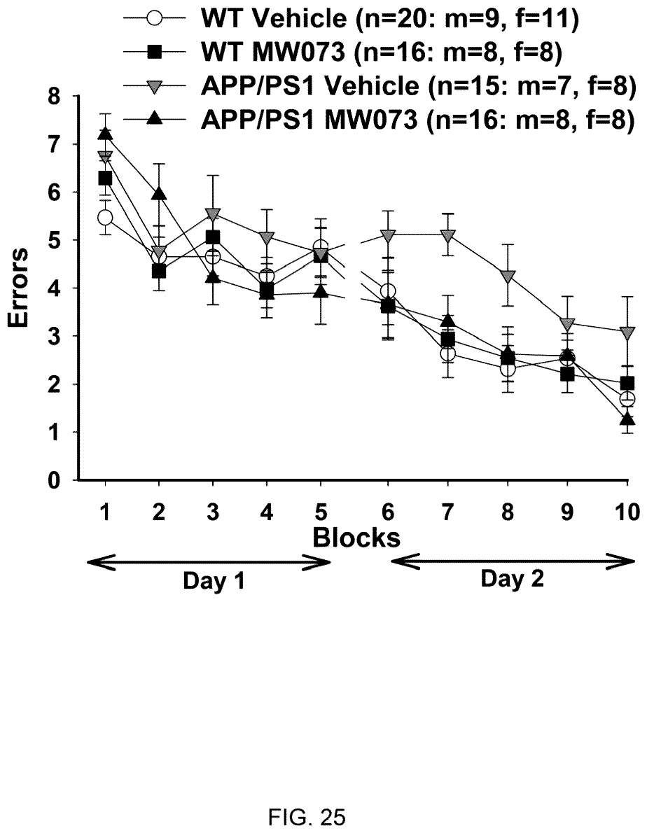

FIG. 25 showing the efficacy of MW073 following intervention at a disease state onto the APP/PS1 mouse model of amyloid elevation using a test of spatial memory, the RAWM. MW073 (5 mg/Kg, o.s., daily, from day 100 until day 130) protects APP/PS1 mice against the impairment of spatial memory, while MW073 alone in WT littermates does not affect performance. RAWM: 2 way-ANOVA for repeated measures among all (day 2): F(3,63)=3.217, p=0.0287. One-way ANOVA for block 10: F(3,63)=2.973, p=0.0399; One-way ANOVA for block 9: F(3,63)=0.8758, p=0.4585 One-way ANOVA for block 8: F(3,63)=2.753, p=0.0498 One-way ANOVA for block 7: F(3,63)=4.876, p=0.0041 One-way ANOVA for block 6: F(3,63)=1.108, p=0.3526. The number of animals “n” is indicated on the graph.

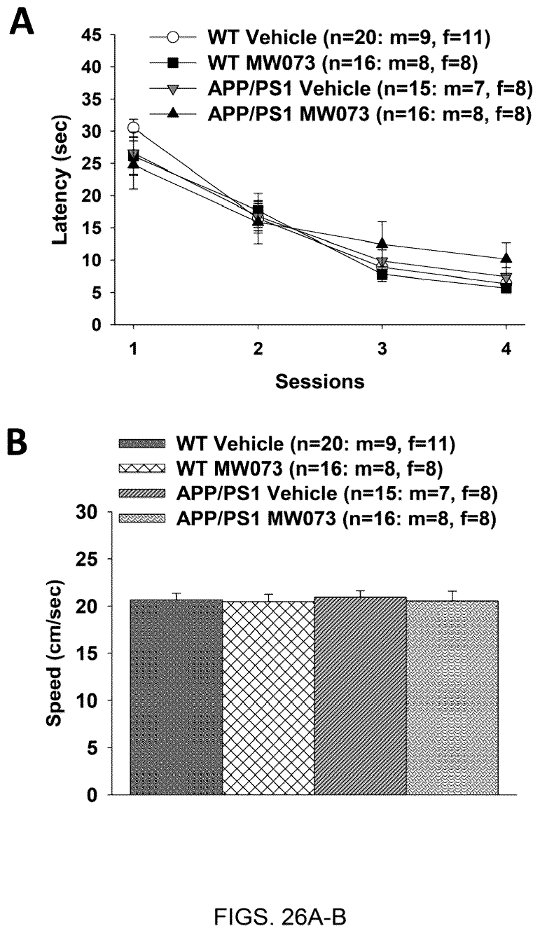

FIG. 26A-B show that MW073 has no effect on vision, motility, or motivation following intervention at a disease state onto the APP/PS1 mouse model of amyloid elevation. Testing with the visible platform task does not reveal any difference in time to reach the visible platform (2-way ANOVA: F(3,63)=0.1220, p=0.9468) (A) and average speed (1-way ANOVA: F(3,63)=0.05784, p=0.9816) (B). MW073 was administered at a concentration of 5 mg/Kg (o.s., daily, from day 100 until day 130). The number of animals “n” is indicated on the graph.

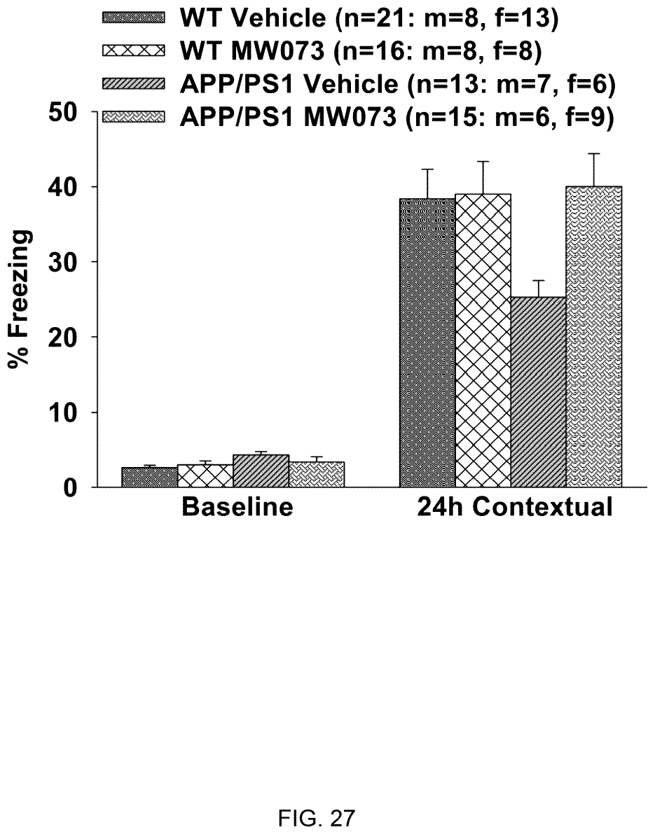

FIG. 27 shows the efficacy of MW073 following intervention at a disease state onto the APP/PS1 mouse model of amyloid elevation using a test of associative memory, the contextual fear conditioning (FC). MW073 (5 mg/Kg, o.s., daily, from day 100 until day 130) protects APP/PS1 mice against the impairment of associative memory, while MW073 alone in WT littermates does not affect memory. FC: ANOVA F(3,61)=2.999, p=0.0374; Bonferroni p=0.0466 WT vehicle vs. APP/PS1 vehicle, p=0.0338 APP/PS1 vehicle vs. APP/PS1 MW073, p>0.999 WT vehicle vs. WT MW073. The number of animals “n” is indicated on the graph.

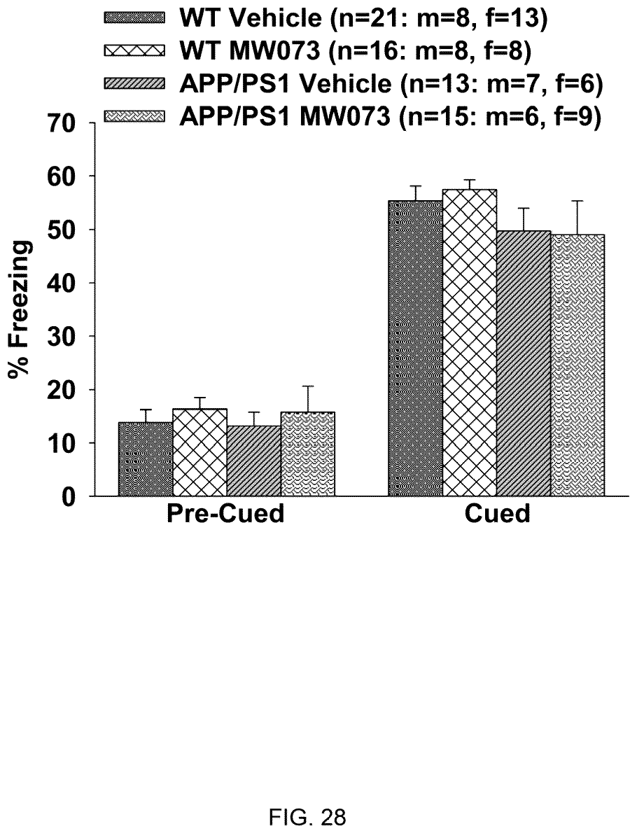

FIG. 28 shows that MW073 has no effect on cued amygdala dependent memory following intervention at a disease state onto the APP/PS1 mouse model of amyloid elevation. Freezing responses during the auditory cued conditioning are not significantly different among the groups (1-way ANOVA: F(3,61)=1.652 p=0.1868). MW073 was administered at a concentration of 5 mg/Kg (o.s., daily, from the age of day 100 until day 130). The number of animals “n” is indicated on the graph.

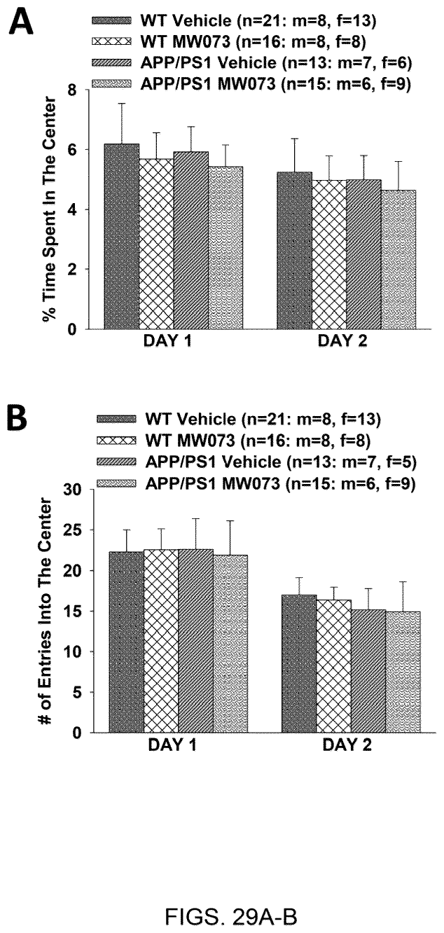

FIG. 29A-B show that MW073 has no effect on exploratory behavior following intervention at a disease state onto the APP/PS1 mouse model of amyloid elevation. Open field test shows a similar percentage of time spent in the center (day 1: F(3,61)=0.1500, p=0.9293; day 2: F(3, 61)=0.1129, p=0.9523) (A) and number of entries into the center among all conditions (day 1: F(3, 61)=0.00894, p=0.9988; day 2: F(3,61)=0.1450, p=0.9325) (B), indicating no differences in exploratory behavior. MW073 was administered at a concentration of 5 mg/Kg (o.s., daily, from day 100 until day 130). The number of animals “n” is indicated on the graph.

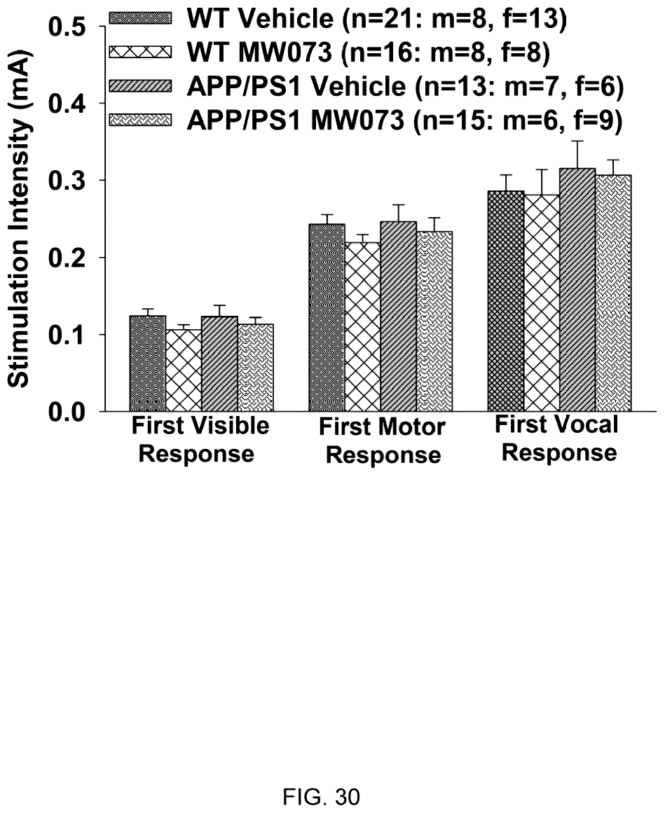

FIG. 30 shows that MW073 has no effect on animal capability of perceiving the electric shock following intervention at a disease state onto the APP/PS1 mouse model of amyloid elevation. No difference is detected among the groups during assessment of the sensory threshold. One-way ANOVA among all: for visible response F(3,61)=0.8108, p=0.4928; for motor response F(3,61)=0.6584, p=0.5808 and for audible response F(3, 61)=0.3801, p=0.7877. MW073 was administered at a concentration of 5 mg/Kg (o.s., daily, from day 100 until day 130). The number of animals “n” is indicated on the graph.

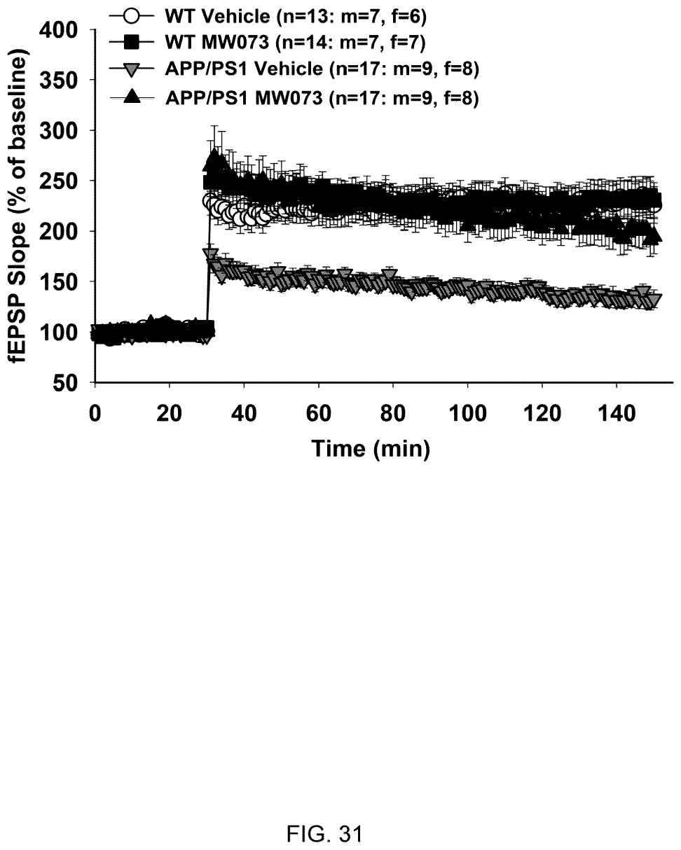

FIG. 31 shows that MW073 has no effect onto the APP/PS1 mouse model of amyloid elevation following intervention at a disease state using LTP as a test of synaptic plasticity. MW073 (5 mg/Kg, o.s., daily, from day 100 until day 140) reverts the LTP defect in the APP/PS1 mouse model of Alzheimer's Disease. MW073 alone in WT littermates does not affect potentiation. 2-way ANOVA F(3,57)=9.256, p<0.0001 ANOVA for repeated measures between groups: F(1,25)=0.1559, p=0.6963 WT vehicle vs WT+MW073. F(1,28)=30.17, p<0.0001 WT vehicle vs. app/ps1 vehicle; F(1,32)=16.03, p=0.0003 app/ps1 vehicle vs. app/ps1+MW073; F(1,29)=0.2725, p=0.6065 WT+MW073 vs. app/ps1+MW073; The number of slices “n” is indicated on the graph.

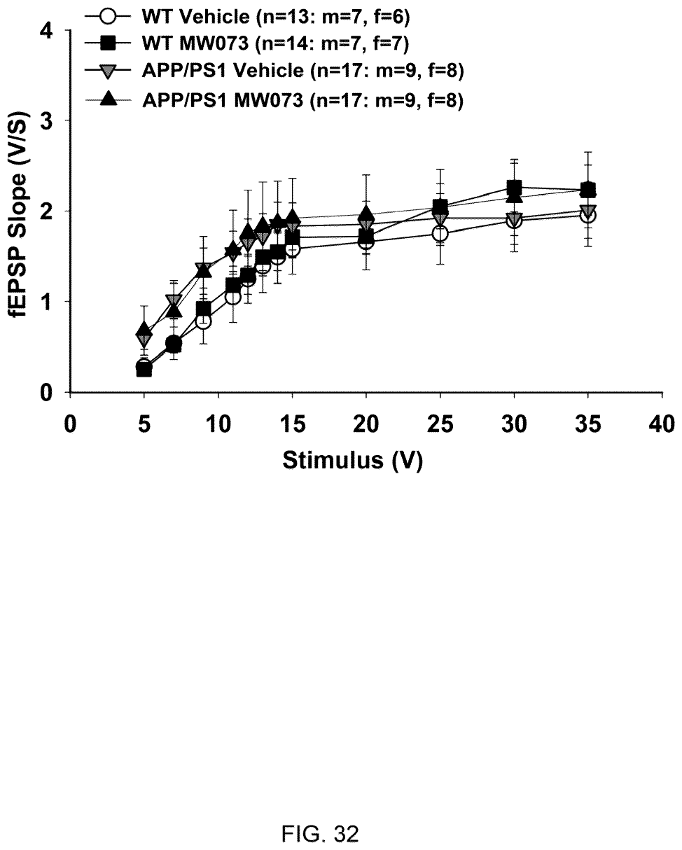

FIG. 32 shows that MW073 has no effect on basal neurotransmission in the APP/PS1 mouse model of amyloid elevation following intervention at a disease state. MW073 (5 mg/Kg, o.s., daily, from day 100 until day 140) does not alter the input/output relationship in a test of basal synaptic transmission. 2-Way-ANOVA: F(3,57)=0.3324, p=0.8019. The number of slices “n” is indicated on the graph.

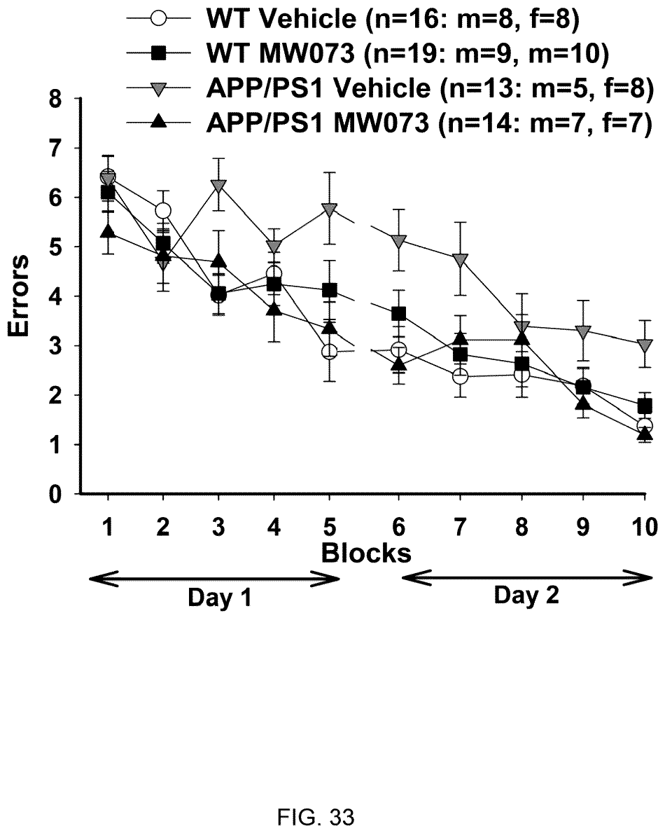

FIG. 33 shows the efficacy of MW073 in a prevention trial onto the APP/PS1 mouse model of amyloid elevation using a test of spatial memory, the RAWM. MW073 (5 mg/Kg, o.s., daily, from day 70 until 100) protects mice against the impairment of spatial memory, while MW073 alone in WT littermates does not affect performance. RAWM: 2 way-ANOVA for repeated measures among all (day 2): F(3,58)=4.907, p=0.0042. One-way ANOVA for block 10: F(3,58)=5.882, p=0.0014; One-way ANOVA for block 9: F(3,58)=1.830, p=0.1518 One-way ANOVA for block 8: F(3,58)=0.6642, p=0.5775 One-way ANOVA for block 7: F(3,58)=3.468, p=0.0218 One-way ANOVA for block 6: F(3,58)=4.539, p=0.0063. The number of animals “n” is indicated on the graph.

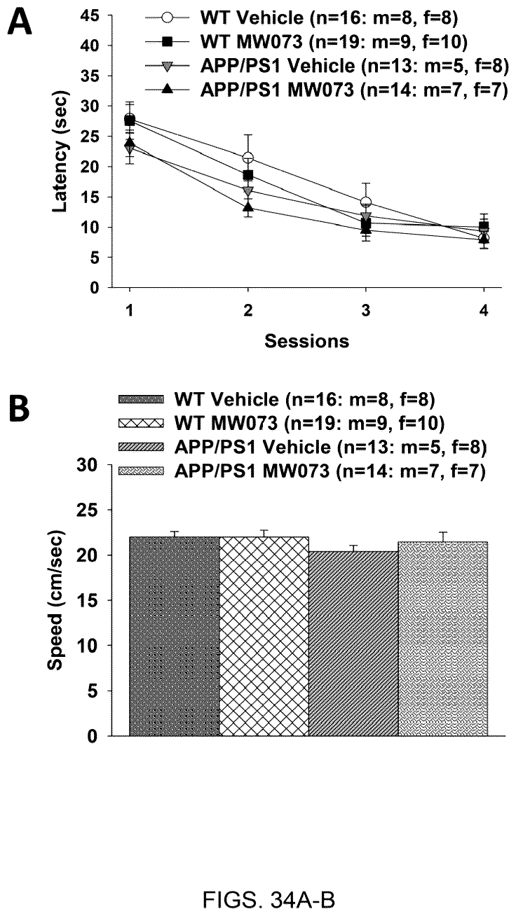

FIG. 34A-B show that MW073 has no effect on vision, motility, or motivation during a prevention trial onto the APP/PS1 mouse model of amyloid elevation. Testing with the visible platform task does not reveal any difference in time to reach the visible platform (2-way ANOVA: F(3,58)=2.512, p=0.0674) (A) and average speed (1-way ANOVA: F(3,58)=0.8268, p=0.4845) (B). MW073 was administered at a concentration of 5 mg/Kg (o.s., daily, from day 70 until 100, 30 min prior the 1st session over 2 days). The number of animals “n” is indicated on the graph.

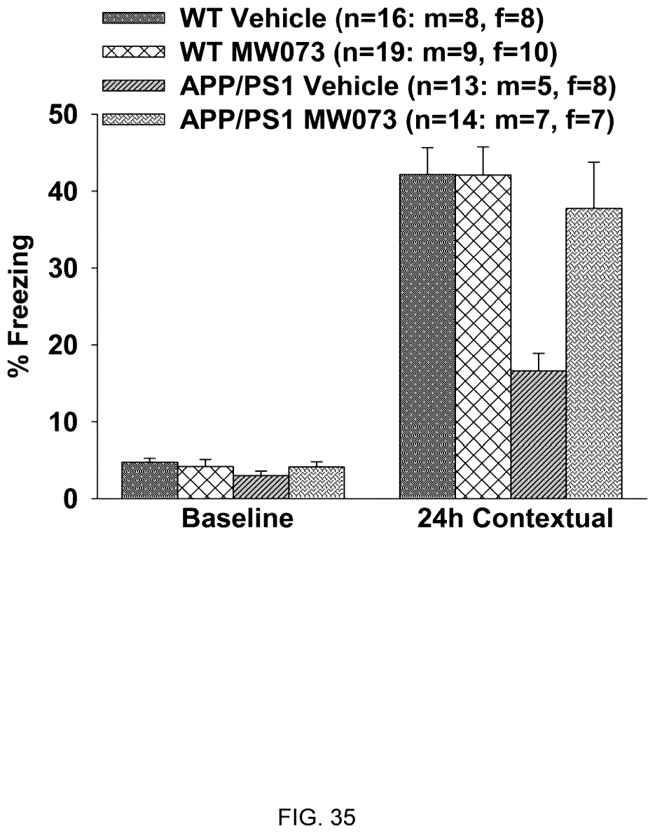

FIG. 35 shows the efficacy of MW073 in a prevention trial onto the APP/PS1 mouse model of amyloid elevation using a test of associative memory, the contextual fear conditioning (FC). MW073 (5 mg/Kg, o.s., daily, from day 70 until 100) protects mice against the impairment of associative memory, while MW073 alone in WT littermates does not affect memory. FC: ANOVA F(5,54)=2.847, p=0.0460; Bonferroni p=0.0503 WT vehicle vs. APP/PS1 vehicle, p=0.0547 APP/PS1 vehicle vs. APP/PS1 MW073, p>0.999 WT vehicle vs. WT MW073. The number of animals “n” is indicated on the graph.

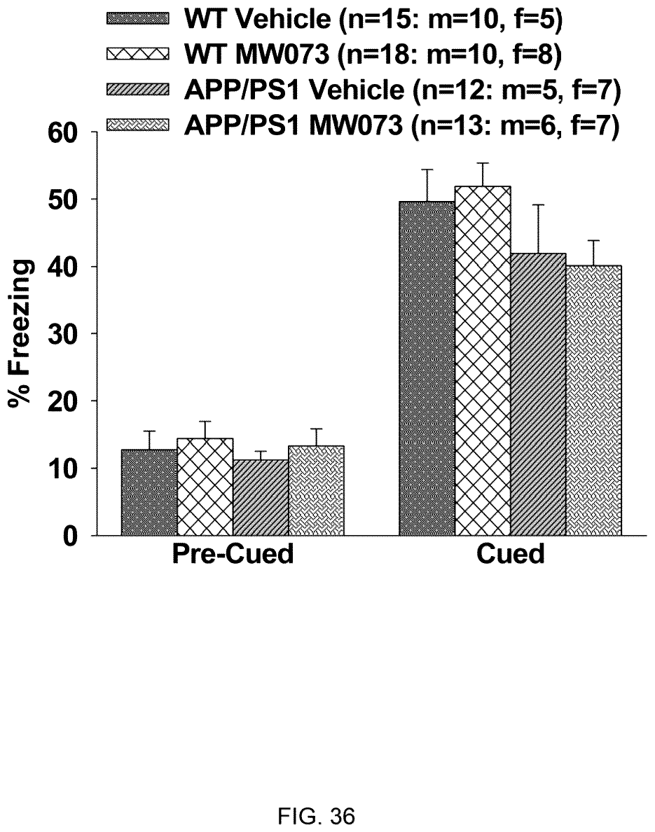

FIG. 36 shows that MW073 has no effect on cued amygdala dependent memory in a prevention trial onto the APP/PS1 mouse model of amyloid elevation. Freezing responses during the auditory cued conditioning are not significantly different among the groups (1-way ANOVA: F(3,54)=1.705 p=0.1770). MW073 was administered at a concentration of 5 mg/Kg (o.s., daily, from day 70 until 100). The number of animals “n” is indicated on the graph.

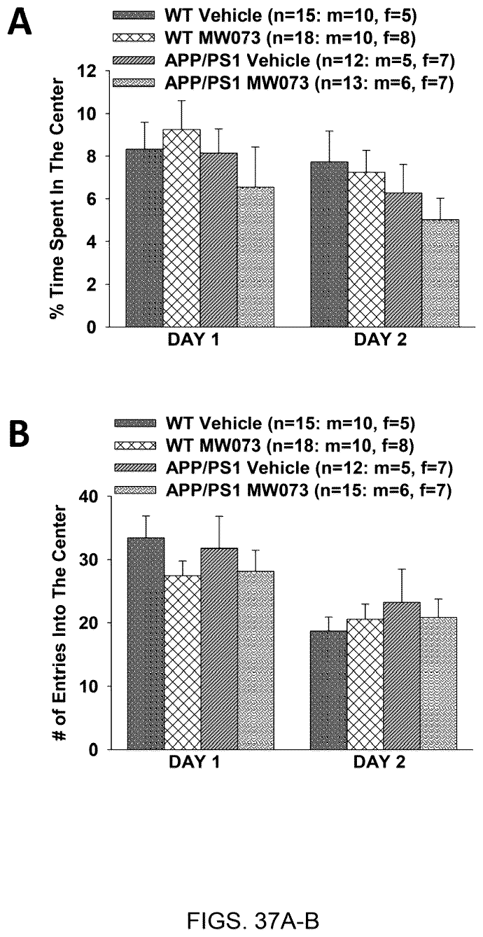

FIG. 37A-B show that MW073 has no effect on exploratory behavior in a prevention trial onto the APP/PS1 mouse model of amyloid elevation. Open field test shows a similar percentage of time spent in the center (day 1: F(3,54)=0.4047, p=0.7502; day 2: F(3, 54)=0.7555, p=0.5240) (A) and number of entries into the center among all conditions (day 1: F(3, 54)=0.6336, p=0.5965; day 2: F(3,54)=0.2847, p=0.8362) (B), indicating no differences in exploratory behaviour. MW073 was administered at a concentration of 5 mg/Kg (o.s., daily, from day 70 until 100). The number of animals “n” is indicated on the graph.

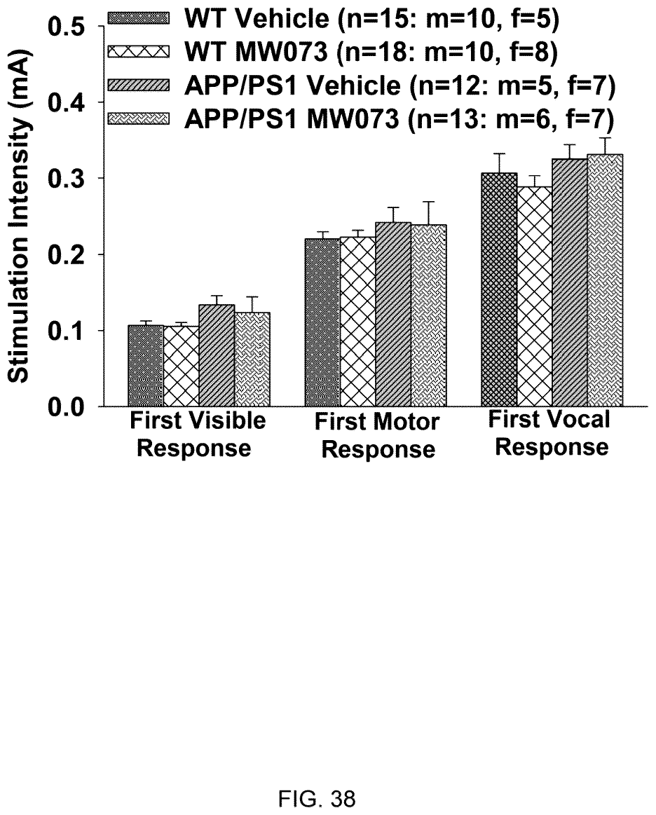

FIG. 38 shows that MW073 has no effect on animal capability of perceiving the electric shock in a prevention trial onto the APP/PS1 mouse model of amyloid elevation. No difference is detected among the groups during assessment of the sensory threshold. One-way ANOVA among all: for visible response F(3,54)=1.528, p=0.2178; for motor response F(3,54)=0.4290, p=0.7330 and for audible response F(3, 54)=02832, p=0.8373. MW073 was administered at a concentration of 5 mg/Kg (o.s., daily, from day 70 until 100). The number of animals “n” is indicated on the graph.

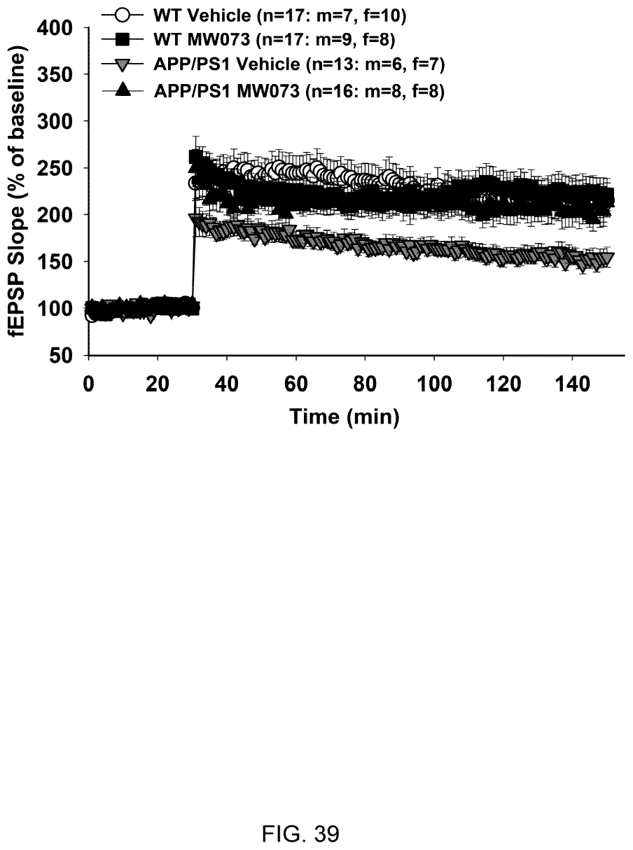

FIG. 39 shows the efficacy of MW073 onto the APP/PS1 mouse model of amyloid elevation in a prevention trial using LTP as a test of synaptic plasticity. MW073 (5 mg/Kg, o.s., daily, from day 70 until 110) prevents the LTP defect in the APP/PS1 mouse model of Alzheimer's Disease. MW073 alone in WT littermates does not affect potentiation. 2-way ANOVA F(3,59)=3.562, p=0.0194 ANOVA for repeated measures between groups: F(1,232)=0.06575, p=0.7993 WT vehicle vs WT+MW073. F(1,28)=9.412, p=0.0047 WT vehicle vs. APP/PS1 vehicle; F(1,27)=8.132, p=0.0082 APP/PS1 vehicle vs. APP/PS1+MW073; F(1,31)=0.4089, p=0.5272 WT+MW073 vs. app/ps1+MW073; The number of slices “n” is indicated on the graph.

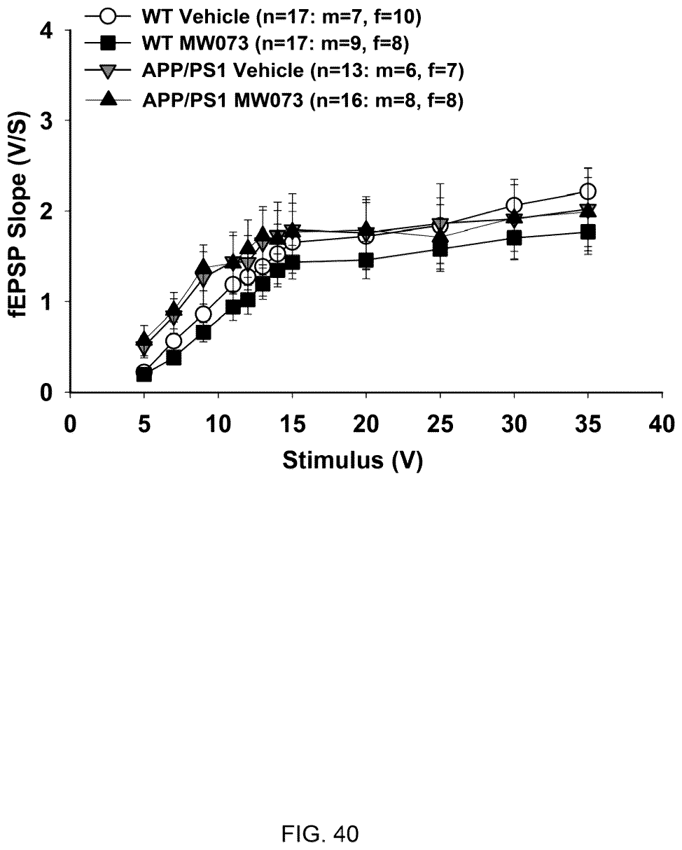

FIG. 40 shows that MW073 has no effect on basal neurotransmission in the APP/PS1 mouse model of amyloid elevation during a prevention trial. MW073 (5 mg/Kg, o.s., daily, from day 70 until day 110) does not alter the input/output relationship in a test of basal synaptic transmission. 2-Way-ANOVA: F(3,59)=0.4881, p=0.6918. The number of slices “n” is indicated on the graph.

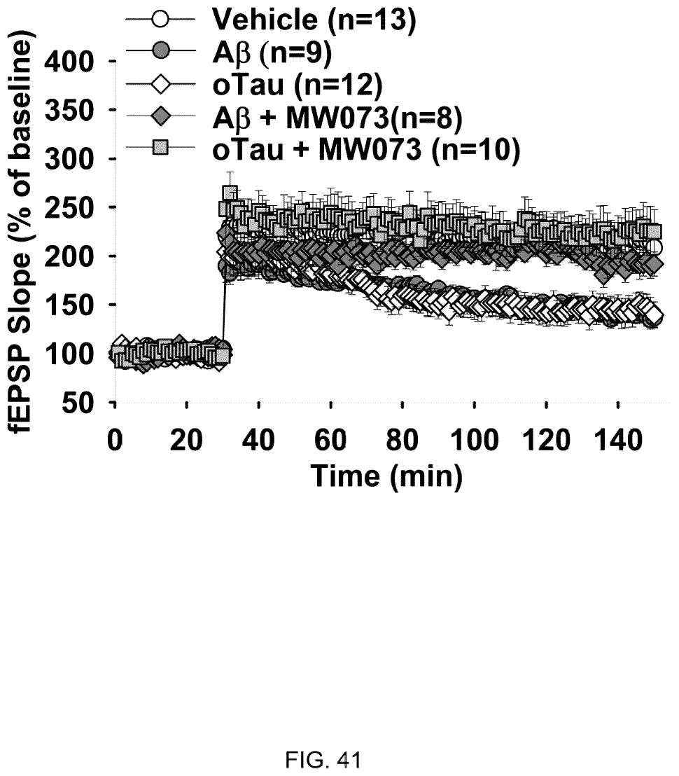

FIG. 41 shows the efficacy of MW073 in AD relevant mouse models characterized by Aβ and tau oligomer elevation using long-term potentiation (LTP) as a test of synaptic plasticity. Perfusion with MW073 (1.9 μM) rescues LTP defect in hippocampal slices treated with 200 nM Aβ- or 50 nM tau-oligomers. Overall ANOVA: F(4,47)=5.542, p=0.001; Two-Way ANOVA among groups: F(1,23)=9.557, p=0.0052 vehicle vs. Tau; F(1,20)=10.17, p=0.0046 vehicle vs. Aβ; F(1,20)=10.13, p=0.0047 Tau vs. Tau+MW073; F(1,15)=10.55, p=0.0054 Aβ vs. Aβ+MW073. The number of slices “n” is indicated in the graph.

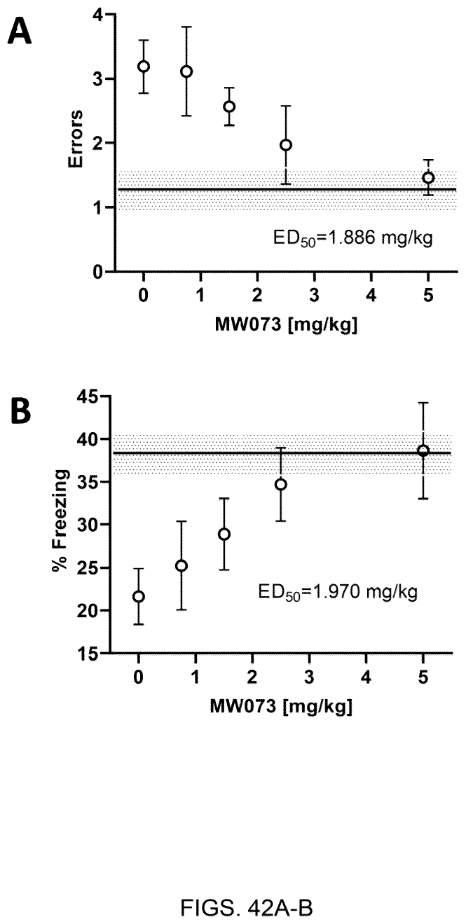

FIG. 42 A-B show the dose response curve for the beneficial effect of MW073 onto radial arm water maze (RAWM) and contextual fear conditioning (FC) defects in APP/PS1 mice. The values of ED50s are indicated in the graphs. Mice were treated with increasing concentrations of MW073 (o.s. daily, for 30-45 days starting from day 60-70). RAWM errors correspond to the number of errors that mice made at the last set of trial (A). Percent freezing corresponds to the percentage of freezing during contextual fear conditioning the day after training with the electric shock (B). For these experiments 9-16 mice per group were tested. The shaded area corresponds to the average number of errors in the RAWM or percent freezing in the contextual FC (continuous lines) and the standard error range in vehicle treated WT mice.

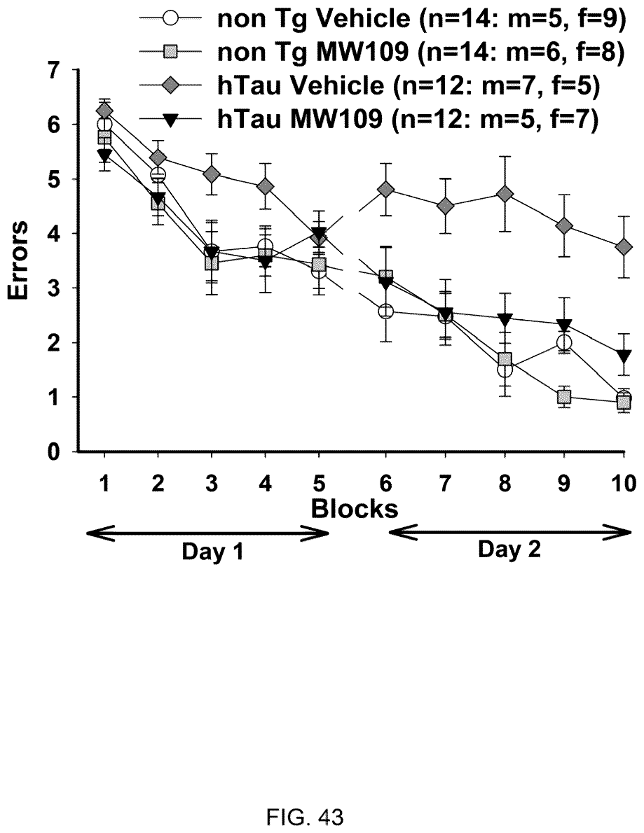

FIG. 43 shows the efficacy of MW109 following intervention at a disease state onto the hTau/Mapt-KO mouse model of tau elevation using a test of spatial memory, the RAWM. MW109 (5 mg/Kg, i.p., daily from the age of 11 months for 45 days) protects hTau/Mapt-KO mice against the impairment of spatial memory, while MW109 alone in control non-transgenic littermates does not affect performance in non-transgenic (nonTg) littermates. RAWM: 2 way-ANOVA for repeated measures among all groups (day 2): F(3,48)=9.320, p<0.0001. One-way ANOVA for block 10: F(3,48)=10.51, p<0.0001; One-way ANOVA for block 9: F(3,48)=6.146, p=0.0013 One-way ANOVA for block 8: F(3,48)=6.801, p=0.0007 One-way ANOVA for block 7: F(3,48)=3.402, p=0.0205. The number of animals “n” is indicated on the graph.

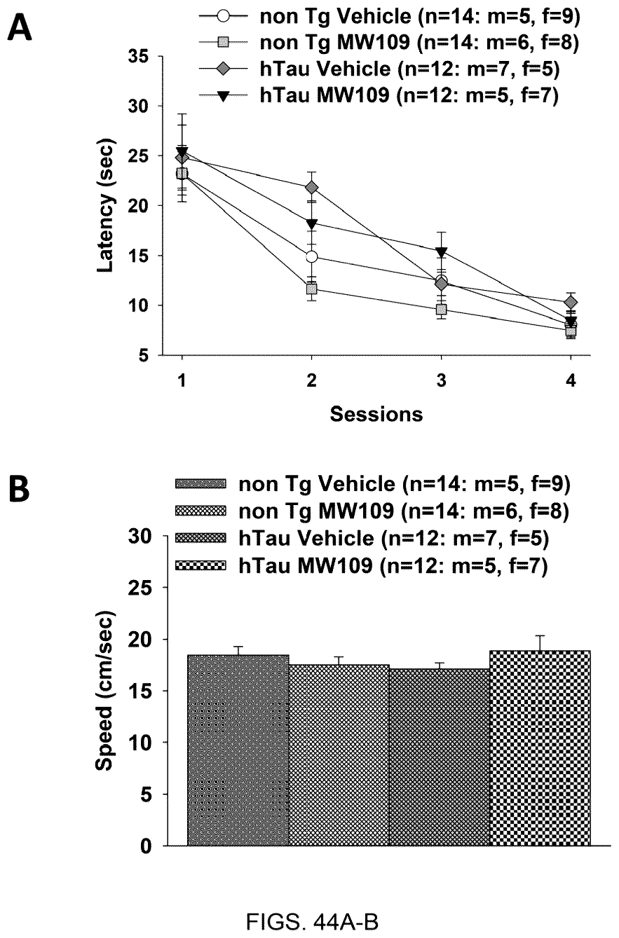

FIG. 44A-B shows that MW109 has no effect on vision, motility, or motivation following intervention at a disease state on the hTau/Mapt-KO mouse model of Alzheimer's disease. Testing with the visible platform task does not reveal any difference in time to reach the visible platform (2-way ANOVA: F(3,48)=2.250, p=0.0945) (A) and average speed (1-way ANOVA: F(3,48)=1.851, p=0.1505) (B). MW109 was administered at a concentration of 5 mg/Kg (i.p., daily, from the age of 11 months for 45 days). The number of animals “n” is indicated on the graph.

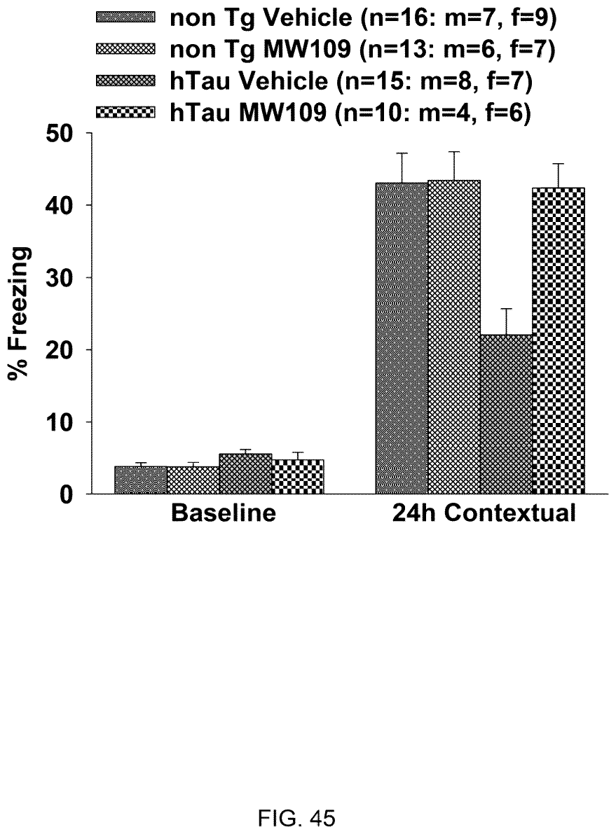

FIG. 45 show the efficacy of MW109 following intervention at a disease state onto the hTau/Mapt-KO mouse model of tau elevation using a test of associative memory, the contextual fear conditioning (FC). MW109 (5 mg/Kg, i.p., daily, from the age of 11 months for 150 days) protects hTau/Mapt-KO mice against the impairment of associative memory, while MW109 alone in control non-transgenic littermates does not affect memory. FC: ANOVA F(3,50)=6.944, p=0.0005; Bonferroni p=0.0010 WT vehicle vs. hTau vehicle, p>0.9999 hTau vehicle vs. hTau MW109, p=0.0045 WT vehicle vs. WT MW109. The number of animals “n” is indicated on the graph.

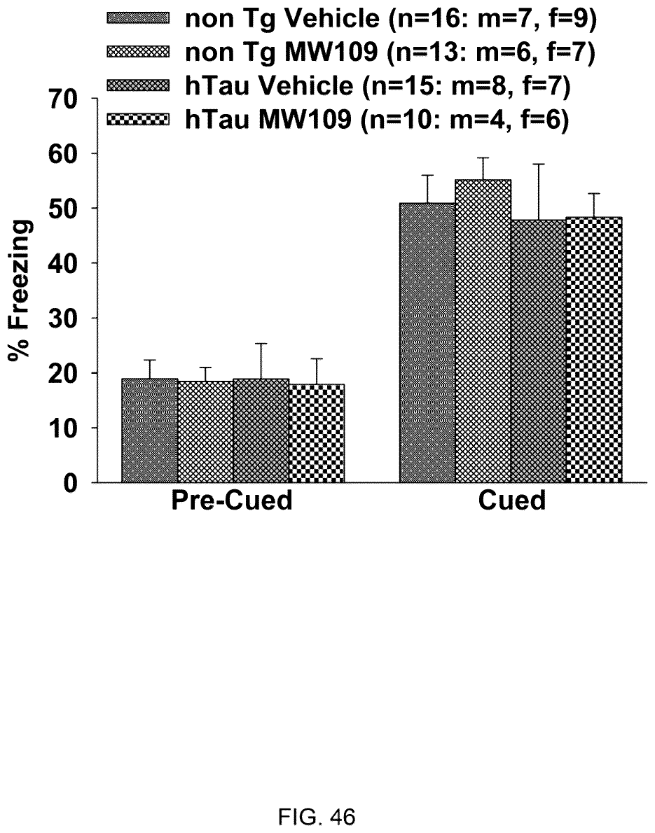

FIG. 46 show that MW109 has no effect on cued amygdala dependent memory following intervention at a disease state onto the hTau/Mapt-KO mouse model of tau elevation. Freezing responses during the auditory cued conditioning are not significantly different among the groups (pre cued: 1-way ANOVA: F(3,50)=0.05398, p=0.9833. cued: 1-way ANOVA: F(3,50)=0.5802, p=0.6308). MW109 was administered at a concentration of 5 mg/Kg (i.p., daily, from the age of 11 months for 150 days). The number of animals “n” is indicated on the graph.

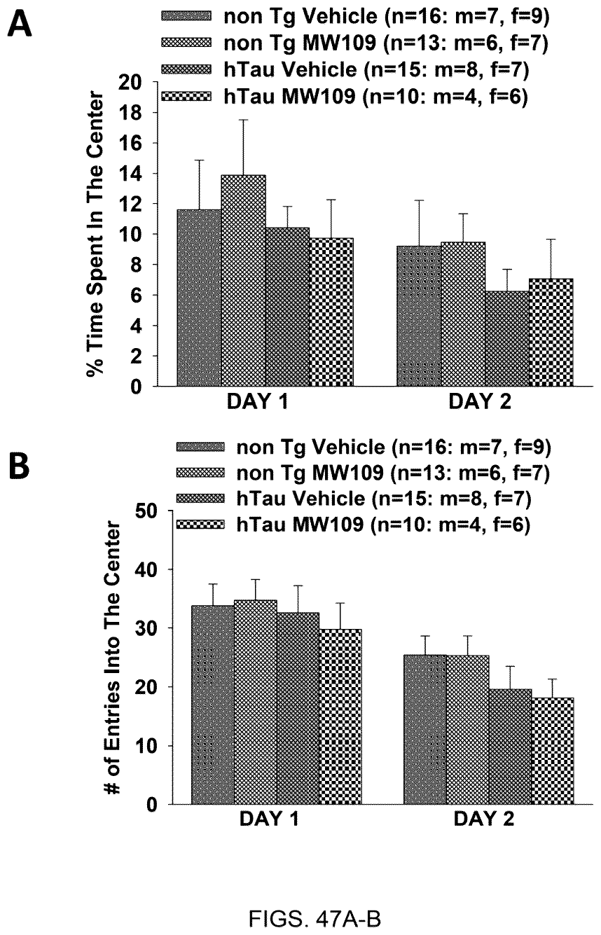

FIG. 47A-B shows that MW109 has no effect on exploratory behavior following intervention at a disease state onto the hTau/Mapt-KO mouse model of tau elevation. Open field test shows a similar percentage of time spent in the center (day 1: F(3,50)=0.3477, p=0.7910; day 2: F(3, 50)=0.4719, p=0.7032) (A) and number of entries into the center among all conditions (day 1: F(3, 50)=0.2431, p=0.8659; day 2: F(3,50)=1.195, p=0.3213) (B), indicating no differences in exploratory behavior. MW109 was administered at a concentration of 5 mg/Kg (i.p., daily, from the age of 11 months for 150 days). The number of animals “n” is indicated on the graph.

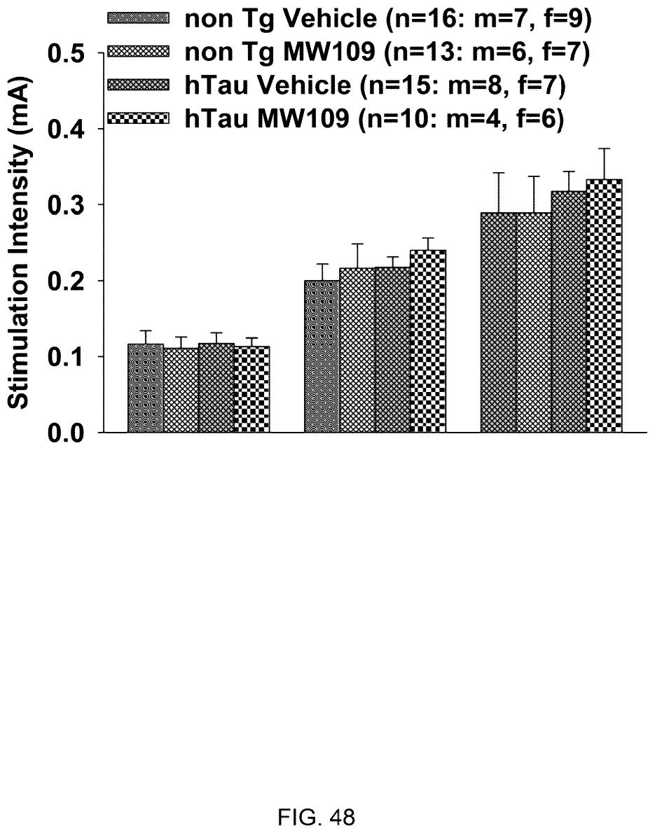

FIG. 48 shows that MW109 has no effect on animal capability of perceiving the electric shock following intervention at a disease state onto the hTau/Mapt-KO mouse model of tau elevation. No difference is detected among the groups during assessment of the sensory threshold. MW109 was administered at a concentration of 5 mg/Kg (i.p., daily, from the age of 11 months for 150 days). One-way ANOVA among all: for visible response F(3,50)=0.04419, p=0.9875; for motor response F(3,50)=0.3138, p=0.8153 and for audible response F(3, 50)=0.4551, p=0.7148. The number of animals “n” is indicated on the graph.

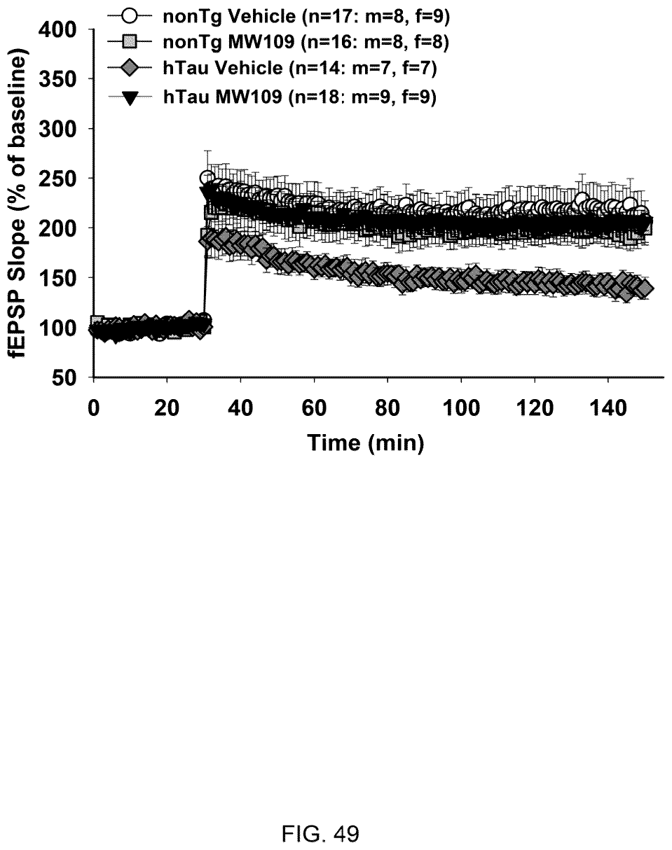

FIG. 49 shows the efficacy of MW109 in the hTau/Mapt-KO mouse model of tau elevation following intervention at a disease state using LTP as a test of synaptic plasticity. MW109 (5 mg/Kg, i.p., daily, from the age of 11 months for 160 days) restores animal capability of undergoing potentiation in the hTau-Mapt-KO mouse model. MW109 alone in control non-transgenic littermates does not affect potentiation. 2-way ANOVA F(3,61)=3.246, p=0.0279 ANOVA for repeated measures between groups: F(1,31)=0.409, p=0.4095 nonTg vehicle vs nonTg+MW109; F(1,29)=7.707, p=0.0095 nonTg vehicle vs. hTau vehicle; F(1,30)=12.46, p=0.0014 hTau vehicle vs. hTau+MW109; F(1,32)=0.08156, p=0.7770 WT+MW109 vs. hTau+MW109; The number of slices “n” is indicated on the graph.

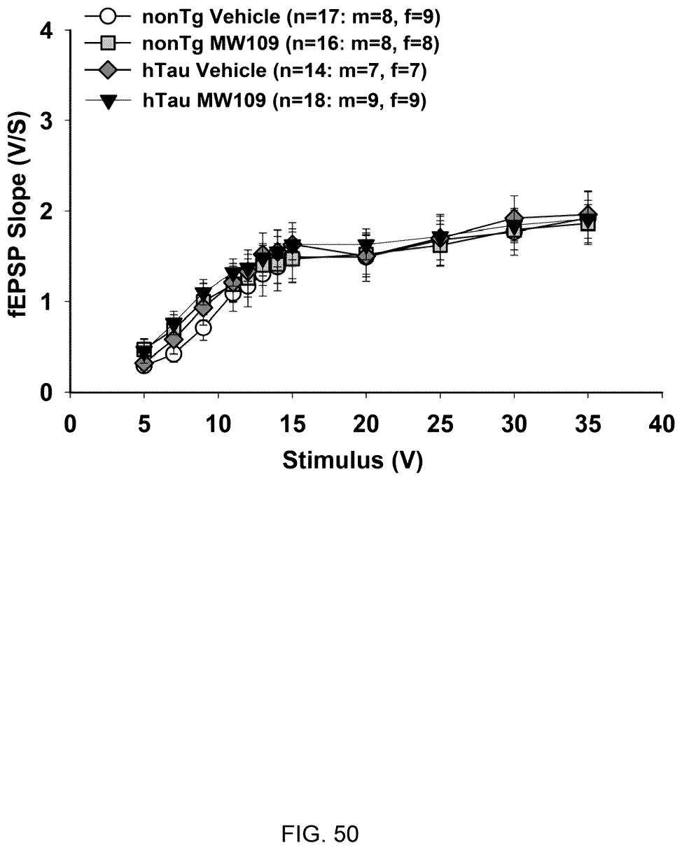

FIG. 50 shows that MW109 has no effect on basal neurotransmission in the hTau/Mapt-KO mouse model of tau elevation following intervention at a disease state. MW109 (5 mg/Kg, i.p., daily, from the age of 11 months for 160 days) does not alter the input/output relationship in a test of basal synaptic transmission. 2-Way-ANOVA: F(3,61)=0.1604, p=0.9226. The number of slices “n” is indicated on the graph.

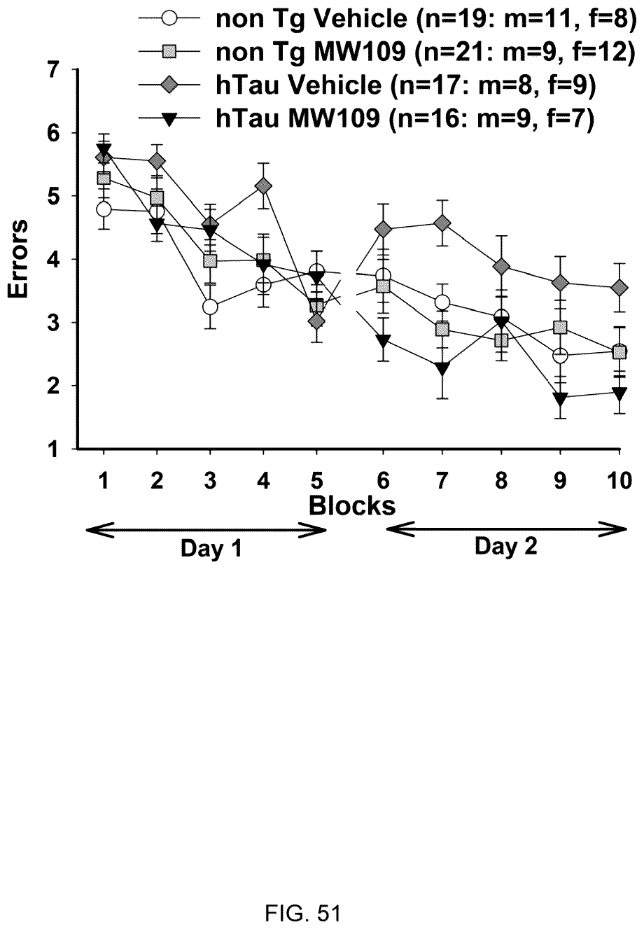

FIG. 51 shows the efficacy of MW109 in a prevention trial onto the hTau/Mapt-KO mouse model of tau elevation using a test of spatial memory, the RAWM. MW109 (5 mg/Kg, i.p., daily, from the age of 7 months for 180 days) protects hTau/Mapt-KO mice against the impairment of spatial memory, while MW109 alone in non-transgenic control littermates does not affect performance. RAWM: 2 way-ANOVA for repeated measures among all (day 2): F(3,69)=4.574, p=0.0056. One-way ANOVA for block 10: F(3,69)=3.110, p=0.0319; One-way ANOVA for block 9: F(3,69)=3.205, p=0.0284 One-way ANOVA for block 8: F(3,69)=1.0905, p=0.3571 One-way ANOVA for block 7: F(3,69)=5.025, p=0.003. The number of animals “n” is indicated on the graph.

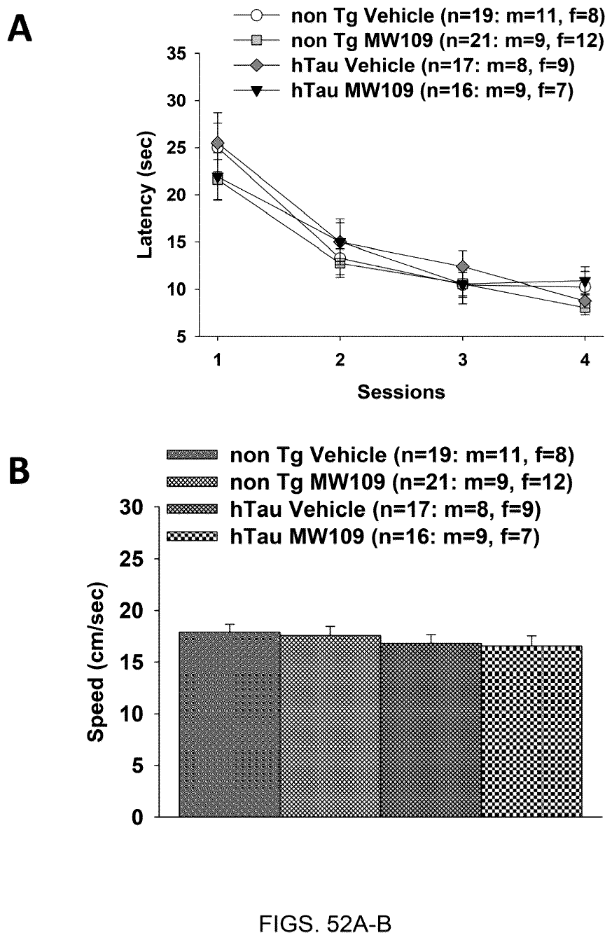

FIG. 52A-B show that MW109 has no effect on vision, motility, or motivation during a prevention trial on the hTau/Mapt-KO mouse model of Alzheimer's disease. Testing with the visible platform task does not reveal any difference in time to reach the visible platform (2-way ANOVA: F(3,69)=0.7249, p=0.5405) (A) and average speed (1-way ANOVA: F(3,69)=0.2225, p=0.8805) (B). MW109 was administered at a concentration of 5 mg/Kg (i.p., daily, from the age of 7 months for 180 days). The number of animals “n” is indicated on the graph.

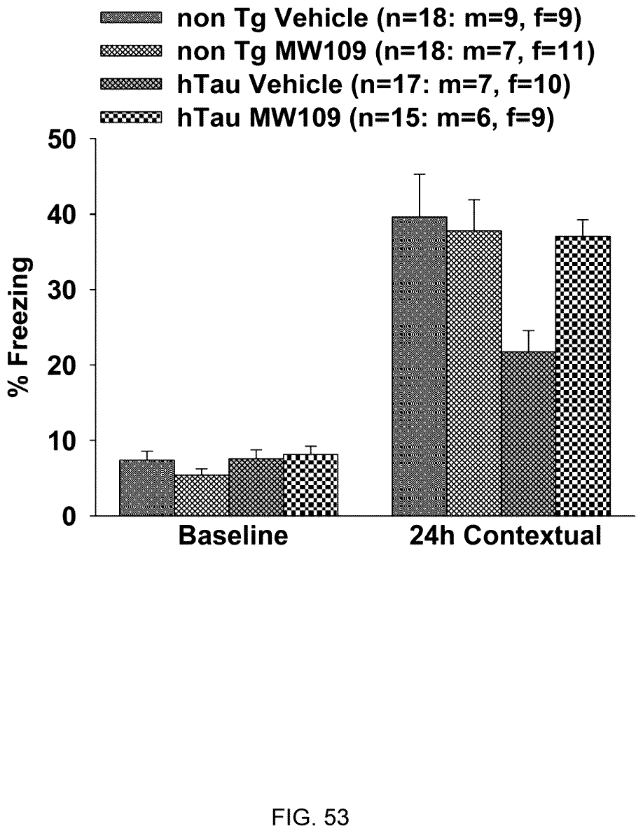

FIG. 53 shows the efficacy of MW109 in a prevention trial onto the hTau/Mapt-KO mouse model of tau elevation using a test of associative memory, the contextual fear conditioning (FC). MW109 (5 mg/Kg, i.p., daily, from the age of 7 months for 240 days) protects hTau/Mapt-KO mice against the impairment of associative memory, while MW109 alone in non-transgenic control littermates does not affect memory. FC: ANOVA F(3,64)=1.372, p=0.2592; Bonferroni p=0.0037 WT vehicle vs. hTau vehicle, p>0.999 hTau vehicle vs. hTau MW109, p=0.0227 WT vehicle vs. WT MW109. The number of animals “n” is indicated on the graph.

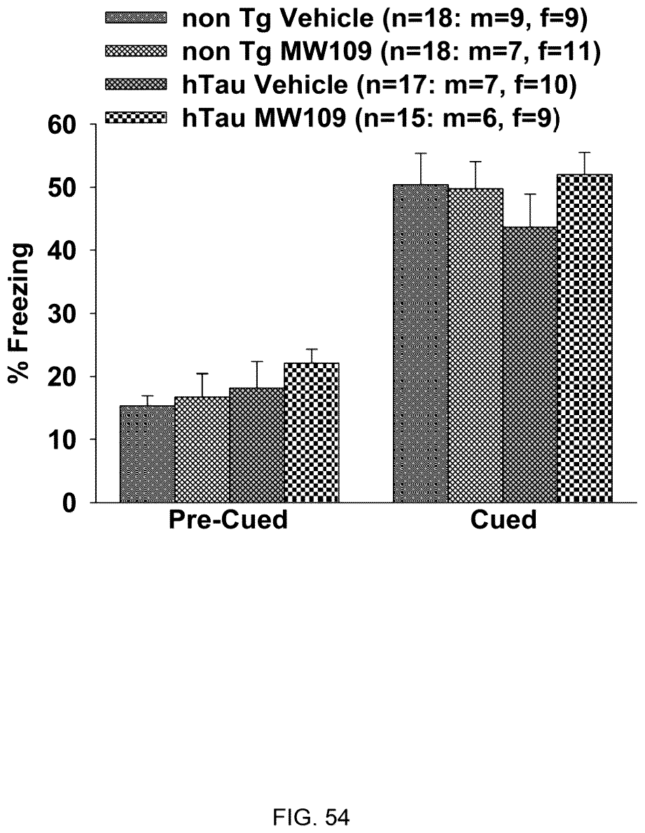

FIG. 54 shows that MW109 has no effect on cued amygdala dependent memory in a prevention trial onto the hTau/Mapt-KO mouse model of tau elevation. Freezing responses during the auditory cued conditioning are not significantly different among the groups (pre cued: 1-way ANOVA: F(3,64)=0.9429, p=0.4253, cued: 1-way ANOVA: F(3,64)=0.3471, p=0.7914). MW109 was administered at a concentration of 5 mg/Kg (i.p., daily, from the age of 7 months for 240 days). The number of animals “n” is indicated on the graph.

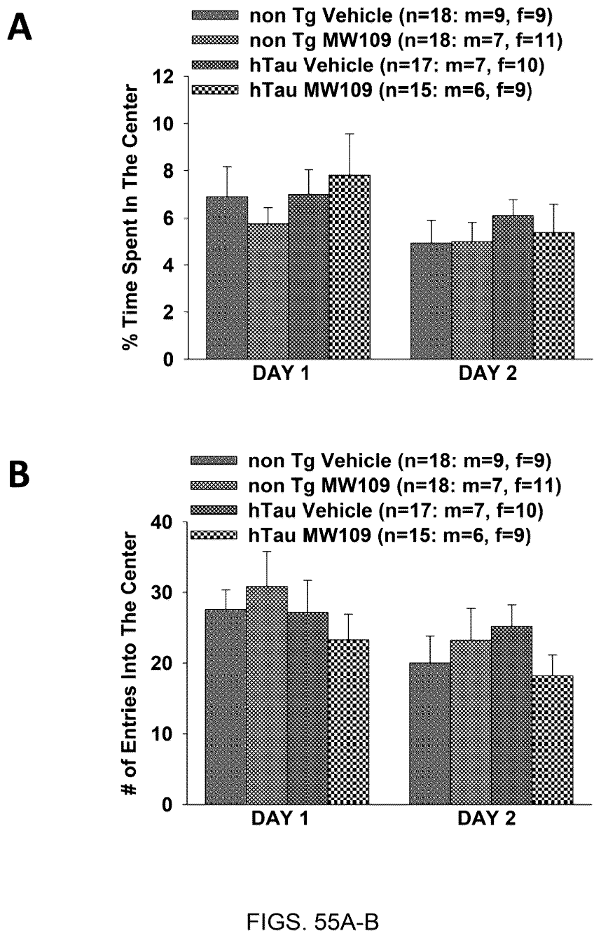

FIG. 55A-B shows that MW109 has no effect on exploratory behavior in a prevention trial onto the hTau/Mapt-KO mouse model of tau elevation. Open field test shows a similar percentage of time spent in the center (day 1: F(3,64)=0.5377, p=0.6581; day 2: F(3, 64)=0.8350, p=0.4796) (Panel A) and number of entries into the center among all conditions (day 1: F(3, 64)=0.5349, p=0.6600; day 2: F(3,64)=0.6798, p=0.5676) (Panel B), indicating no differences in exploratory behavior. MW109 was administered at a concentration of 5 mg/Kg (i.p., daily, from the age of 7 months for 240 days). The number of animals “n” is indicated on the graph.

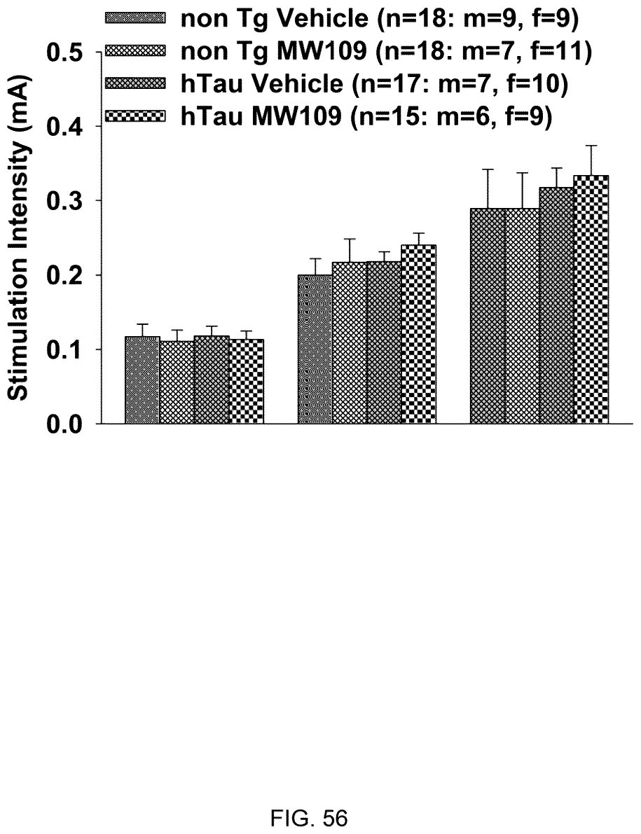

FIG. 56 shows that MW109 has no effect on animal capability of perceiving the electric shock in a prevention trial onto the hTau/Mapt-KO mouse model of tau elevation. No difference is detected among the groups during assessment of the sensory threshold. One-way ANOVA among all: for visible response F(3,64)=0.1200, p=0.9480; for motor response F(3,64)=1.507, p=0.2212 and for audible response F(3, 64)=0.6633, p=0.5777. MW109 was administered at a concentration of 5 mg/Kg (i.p., daily, from the age of 7 months for 240 days). The number of animals “n” is indicated on the graph.

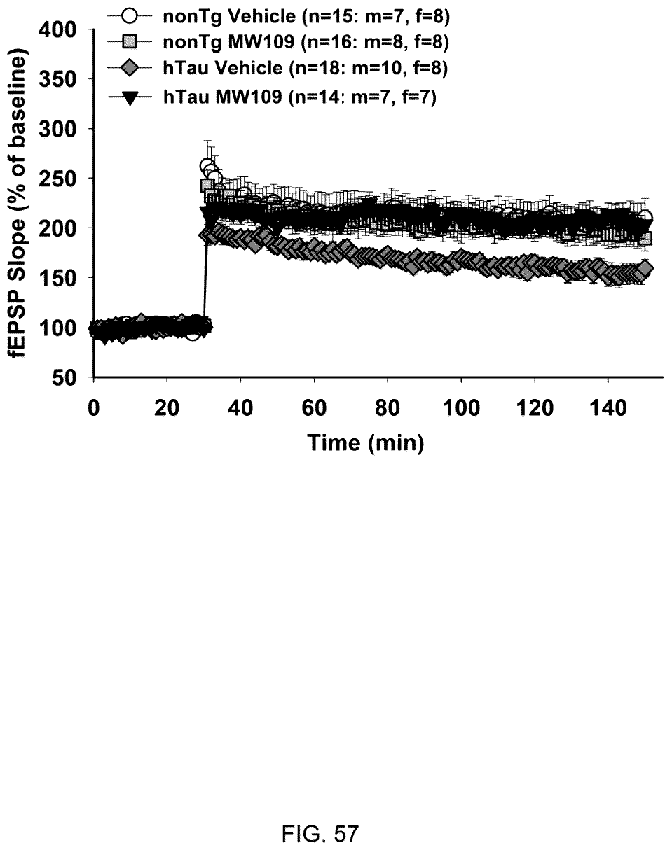

FIG. 57 shows the efficacy of MW109 in the hTau/Mapt-KO mouse model of tau elevation in a prevention trial using LTP as a test of synaptic plasticity. MW109 (5 mg/Kg, i.p., daily, from the age of 7 months for 250 days) prevents the LTP defect in the hTau-Mapt-KO mouse model of Alzheimer's Disease. MW109 alone in non-transgenic control littermates does not affect potentiation. 2-way ANOVA F(3,59)=3.351, p=0.0249 ANOVA for repeated measures between groups: F(1,29)=0.1025, p=0.7511 nonTg vehicle vs nonTg+MW109. F(1,31)=6,093, p=0.0193 nonTg vehicle vs. hTau vehicle; F(1,30)=13.80, p=0.0008 hTau veh vs. hTau MW109; F(1,28)=0.04680, p=0.8303 nonTg MW109 vs. hTau MW109. The number of slices “n” is indicated on the graph.

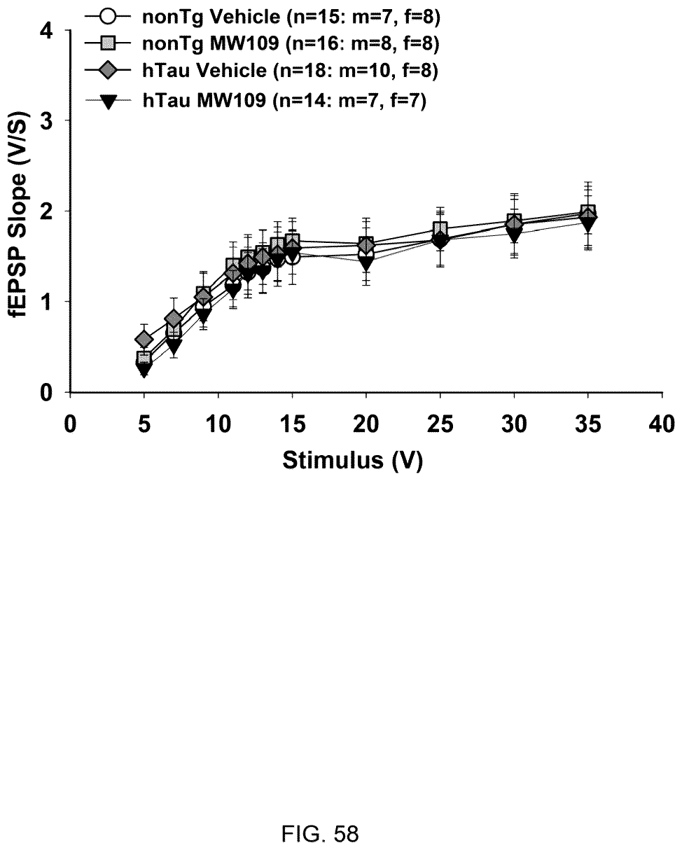

FIG. 58 shows that MW109 has no effect on basal neurotransmission in the hTau/Mapt-KO mouse model of tau elevation during a prevention trial. MW109 (5 mg/Kg, o.s., daily, from the age of 7 months for 250 days) does not alter the input/output relationship in a test of basal synaptic transmission. 2-Way-ANOVA: F(3,59)=0.09577, p=0.9621. The number of slices “n” is indicated on the graph.

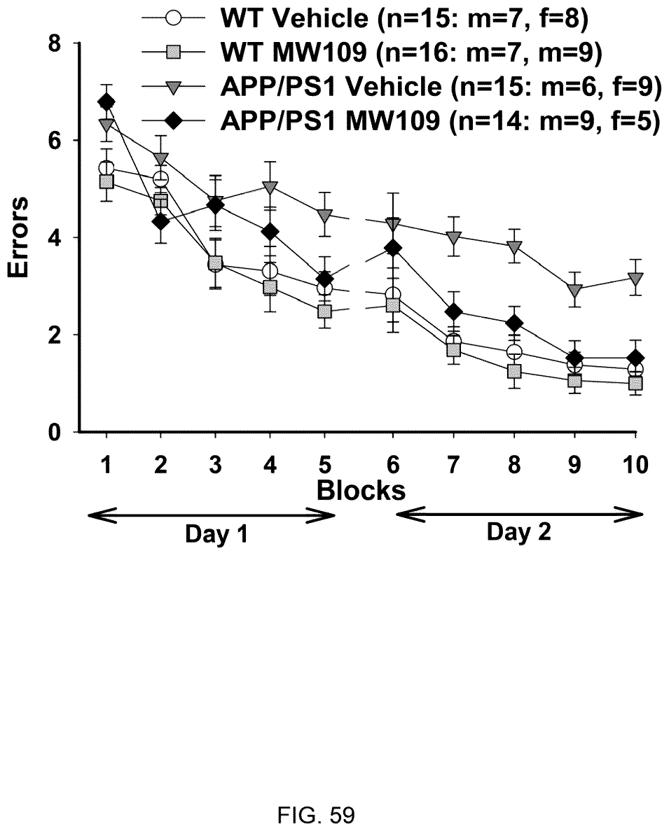

FIG. 59 showing the efficacy of MW109 following intervention at a disease state onto the APP/PS1 mouse model of amyloid elevation using a test of spatial memory, the RAWM. MW109 (5 mg/Kg, i.p., daily, from day 100 until day 130) protects APP/PS1 mice against the impairment of spatial memory, while MW109 alone in WT littermates does not affect performance. RAWM: 2 way-ANOVA for repeated measures among all (day 2): F(3,54)=3.126, p=0.0332. One-way ANOVA for block 10: F(3,54)=5.806, p=0.0016; One-way ANOVA for block 9: F(3,54)=3.580, p=0.0196 One-way ANOVA for block 8: F(3,54)=2,052 p=0.1175 One-way ANOVA for block 7: F(3,54)=1.154, p=0.3357. The number of animals “n” is indicated on the graph.

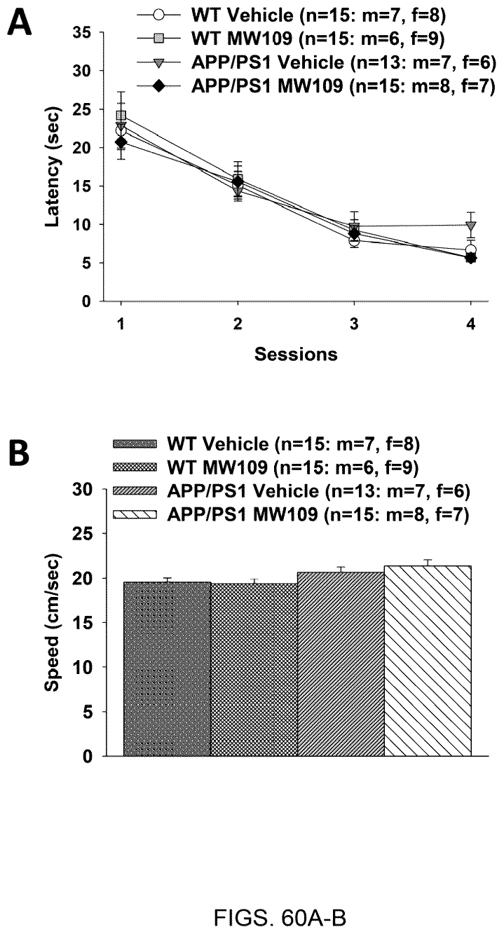

FIG. 60A-B show that MW109 has no effect on vision, motility, or motivation following intervention at a disease state onto the APP/PS1 mouse model of amyloid elevation. Testing with the visible platform task does not reveal any difference in time to reach the visible platform (2-way ANOVA: F(3,54)=0.3810, p=0.7671) (Panel A) and average speed (1-way ANOVA: F(3,54)=2.664, p=0.0570) (Panel B). MW109 was administered at a concentration of 5 mg/Kg (i.p., daily, from day 100 until day 130). The number of animals “n” is indicated on the graph.

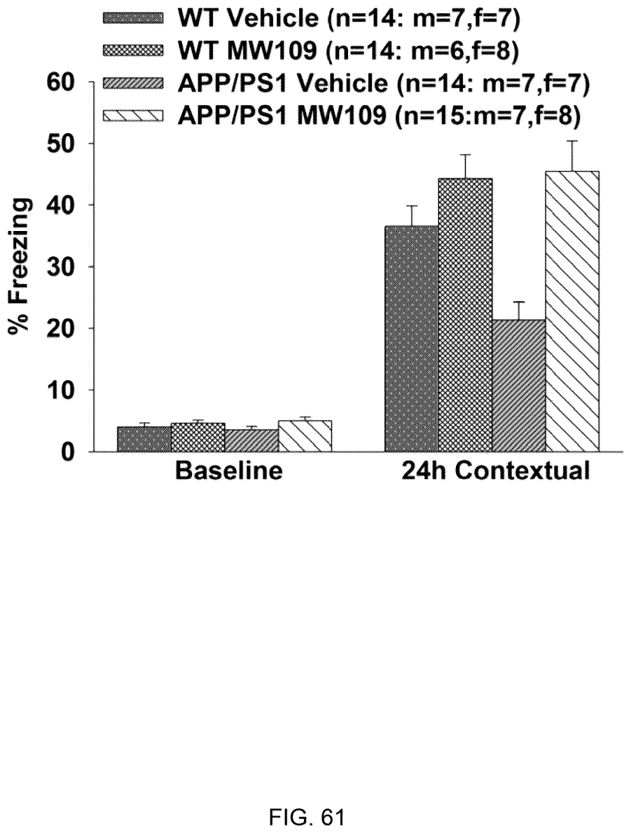

FIG. 61 shows the efficacy of MW109 following intervention at a disease state onto the APP/PS1 mouse model of amyloid elevation using a test of associative memory, the contextual fear conditioning (FC). MW109 (5 mg/Kg, i.p., daily, from day 100 until day 130) protects APP/PS1 mice against the impairment of associative memory, while MW109 alone in WT littermates does not affect memory. FC: ANOVA F(3,53)=10.02, p, 0.001; Bonferroni p=0.0097 WT vehicle vs. app/ps1 vehicle, p<0.0001 app/ps1 vehicle vs. APP/PS1 MW109, p=0.3161 WT vehicle vs. WT MW109. The number of animals “n” is indicated on the graph.

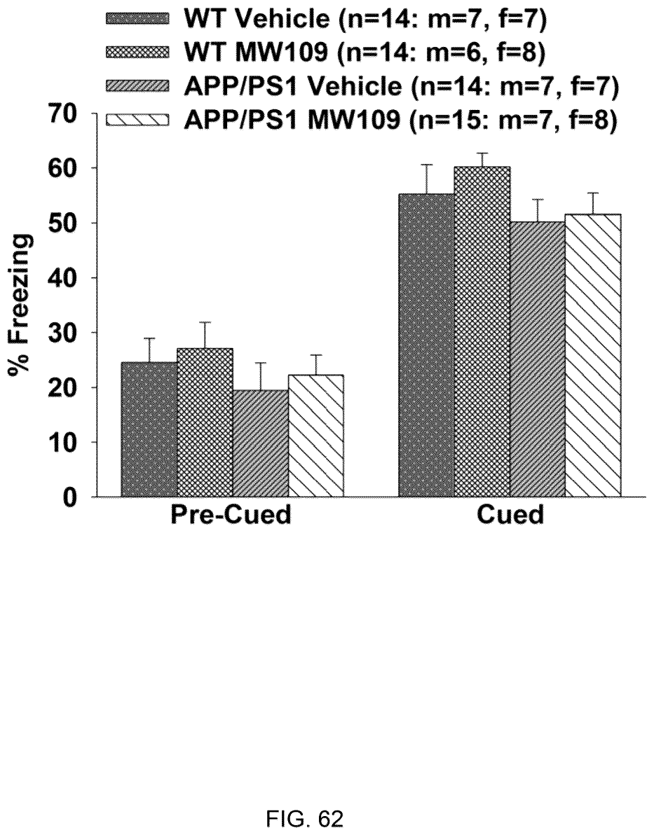

FIG. 62 shows that MW109 has no effect on cued amygdala dependent memory following intervention at a disease state onto the APP/PS1 mouse model of amyloid elevation. Freezing responses during the auditory cued conditioning are not significantly different among the groups (pre-cued: 1-way ANOVA: F(3,53)=0.5241 p=0.6676 and cued: 1-way ANOVA F(3,53)=1.178 p=0.3271). MW109 was administered at a concentration of 5 mg/Kg (i.p., daily, from the age of day 100 until day 130). The number of animals “n” is indicated on the graph.

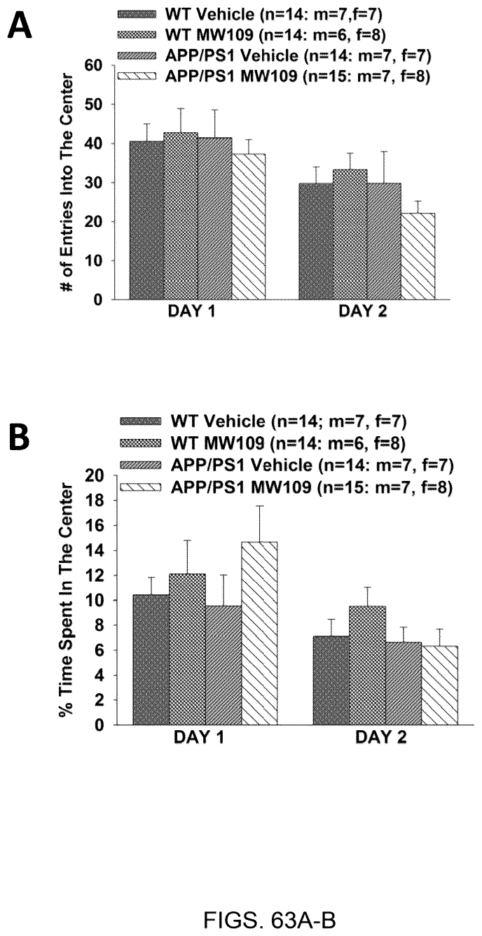

FIG. 63A-B show that MW109 has no effect on exploratory behavior following intervention at a disease state onto the APP/PS1 mouse model of amyloid elevation. Open field test shows a similar percentage of time spent in the center (day 1: F(3,53)=0.8126, p=0.4926; day 2: F(3,53)=0.7270, p=0.5404) (Panel A) and number of entries into the center among all conditions (day 1: F(3,53)=0.2360, p=0.8709; day 2: F(3,53)=0.8091, p=0.4945) (Panel B), indicating no differences in exploratory behavior. MW109 was administered at a concentration of 5 mg/Kg (i.p., daily, from day 100 until day 130). The number of animals “n” is indicated on the graph.

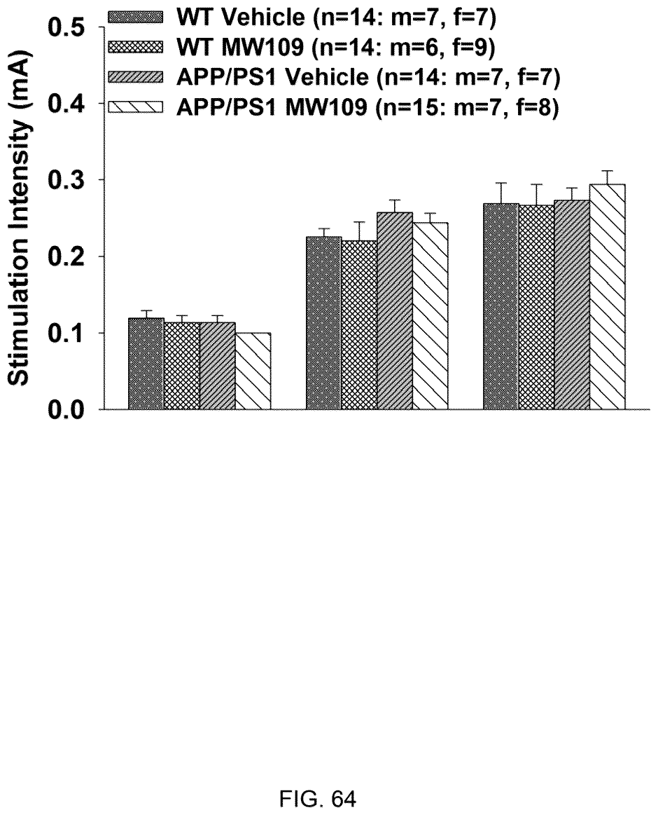

FIG. 64 shows that MW109 has no effect on animal capability of perceiving the electric shock following intervention at a disease state onto the APP/PS1 mouse model of amyloid elevation. No difference is detected among the groups during assessment of the sensory threshold. One-way ANOVA among all: for visible response F(3,53)=1.083, p=0.3644; for motor response F(3,53)=0.8831, p=0.4558 and for audible response F(3,53)=0.1454, p=0.9355. MW073 was administered at a concentration of 5 mg/Kg (i.p., daily, from day 100 until day 130). The number of animals “n” is indicated on the graph.

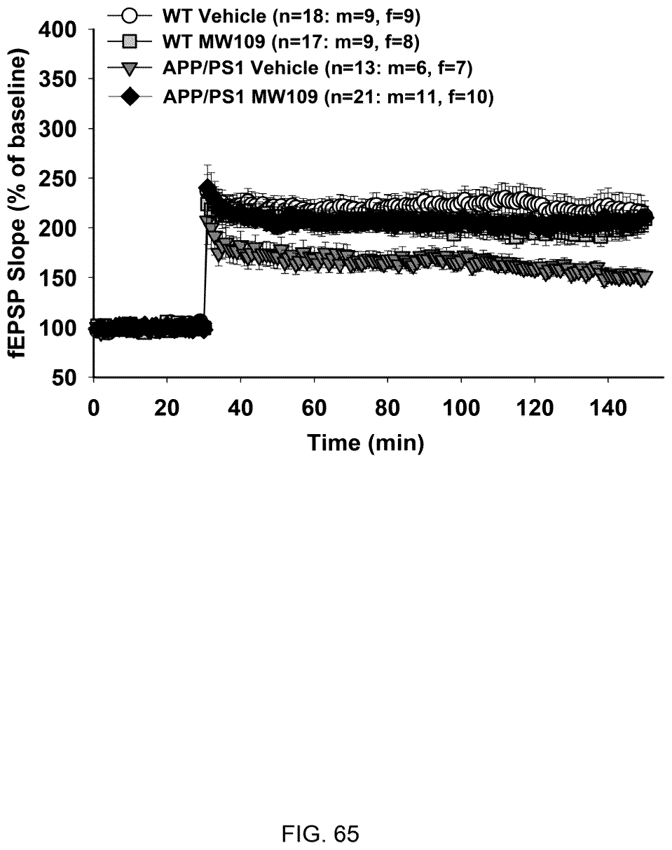

FIG. 65 shows that MW109 has no effect onto the APP/PS1 mouse model of amyloid elevation following intervention at a disease state using LTP as a test of synaptic plasticity. MW109 (5 mg/Kg, i.p., daily, from day 100 until day 140) restores animal capability of undergoing potentiation in APP/PS1 mice. MW109 alone in control WT mice does not affect potentiation. 2-way ANOVA F(3,65)=4.171, p=0.0092 ANOVA for repeated measures between groups: F(1,33)=0.8916, p=0.3519 WT vehicle vs WT+MW109; F(1,29)=11.18, p=0.0023 WT vehicle vs. APP/PS1 vehicle; F(1,32)=10.31, p=0.0030 APP/PS1 vehicle vs. APP/PS1+MW109; F(1,36)=0.02473, p=0.8759 WT+MW109 vs. APP/PS1+MW109; The number of slices “n” is indicated on the graph.

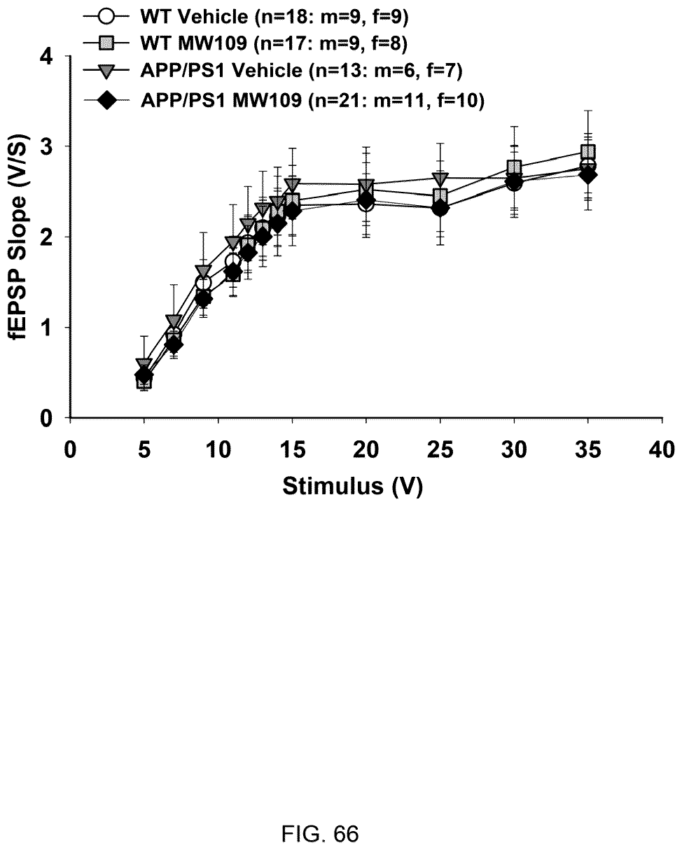

FIG. 66 shows that MW109 has no effect on basal neurotransmission in the APP/PS1 mouse model of amyloid elevation following intervention at a disease state. MW109 (5 mg/Kg, 5 mg/Kg, i.p., daily, from day 100 until day 140) does not alter the input/output relationship in a test of basal synaptic transmission. 2-Way-ANOVA: F(3,65)=0.09773, p=0.9610. The number of slices “n” is indicated on the graph.

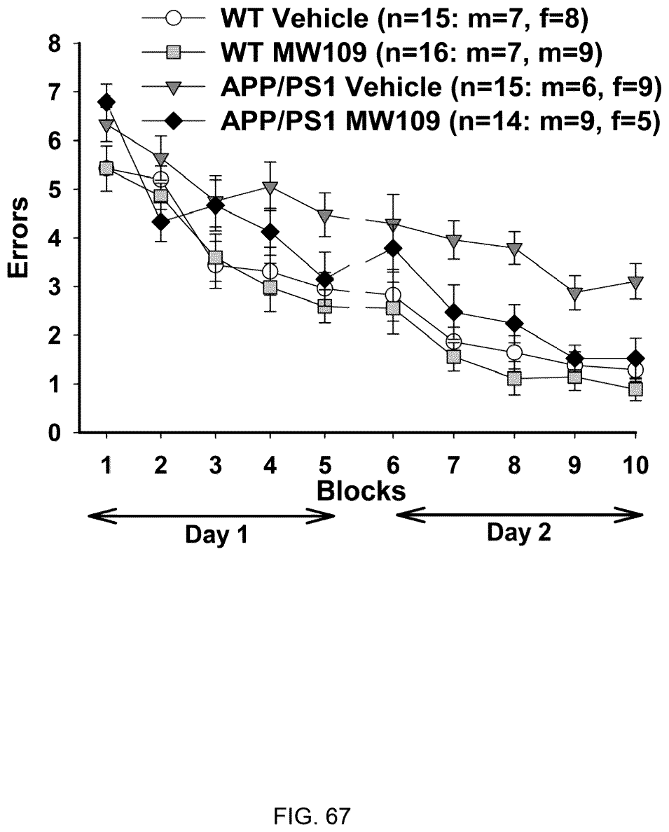

FIG. 67 shows the efficacy of MW109 in a prevention trial onto the APP/PS1 mouse model of amyloid elevation using a test of spatial memory, the RAWM. MW109 (5 mg/Kg, i.p., daily, from day 70 until 100) protects mice against the impairment of spatial memory, while MW109 alone in WT littermates does not affect performance. RAWM: 2 way-ANOVA for repeated measures among all (day 2): F(3,56)=7.822, p=0.0002. One-way ANOVA for block 10: F(3,56)=7.763, p=0.0002; One-way ANOVA for block 9: F(3,56)=6.396, p=0.0008 One-way ANOVA for block 8: F(3,56)=9.186, p<0.0001 One-way ANOVA for block 7: F(3,56)=6.385, p=0.0008.

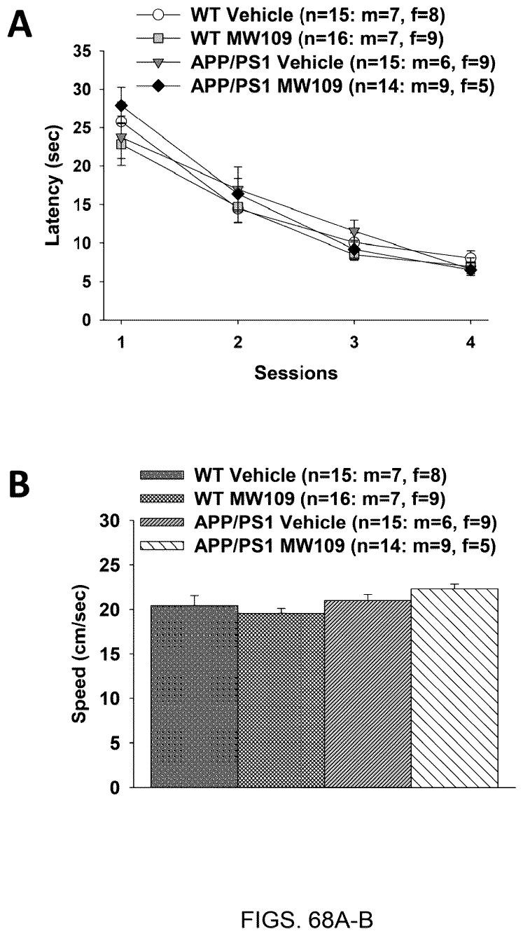

FIG. 68A-B show that MW109 has no effect on vision, motility, or motivation during a prevention trial onto the APP/PS1 mouse model of amyloid elevation. Testing with the visible platform task does not reveal any difference in time to reach the visible platform (2-way ANOVA: F(3,56)=0.5132, p=0.6748) (A) and average speed (1-way ANOVA: F(3,56)=2.262, p=0.0912) (B). MW0109 was administered at a concentration of 5 mg/Kg (i.p., daily, from day 70 until 100). The number of animals “n” is indicated on the graph.

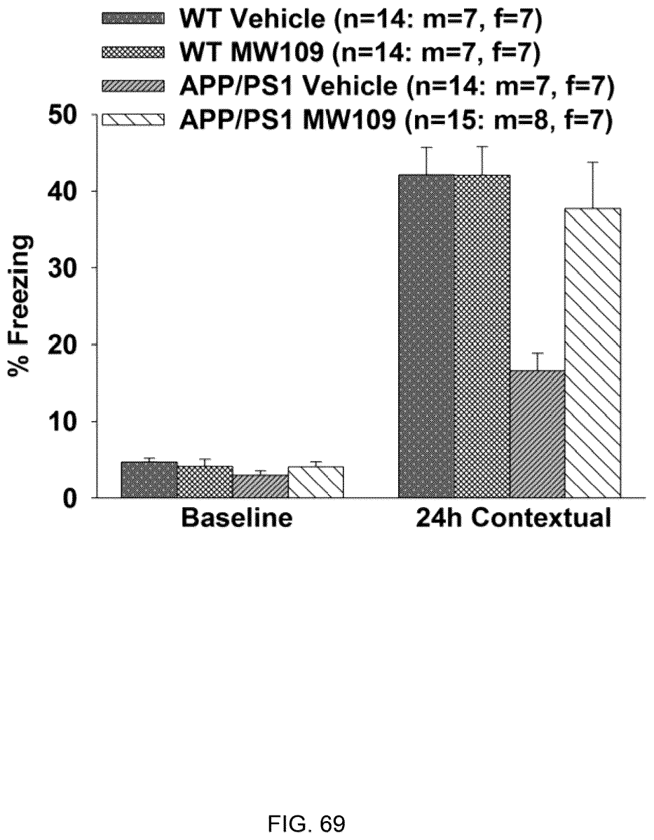

FIG. 69 shows the efficacy of MW109 in a prevention trial onto the APP/PS1 mouse model of amyloid elevation using a test of associative memory, the contextual fear conditioning (FC). MW109 (5 mg/Kg, i.p., daily, from day 70 until 100) protects mice against the impairment of associative memory, while MW109 alone in WT littermates does not affect memory. FC: ANOVA F(3,53)=8.349, p=0.0001; Bonferroni p=0.0002 WT vehicle vs. app/ps1 vehicle, p=0.002 app/ps1 vehicle vs. app/ps1 MW109, p>0.999 WT vehicle vs. WT MW109. The number of animals “n” is indicated on the graph.

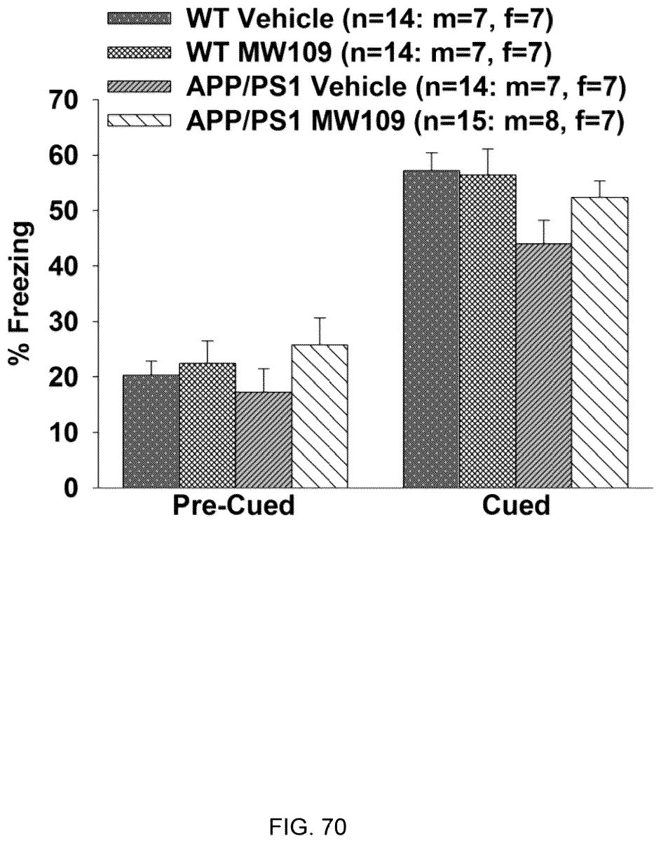

FIG. 70 shows that MW109 has no effect on cued amygdala dependent memory in a prevention trial onto the APP/PS1 mouse model of amyloid elevation. Freezing responses during the auditory cued conditioning are not significantly different among the groups (pre cued: 1-way ANOVA: F(3,53)=0.7843 p=0.5080, cued: 1-way ANOVA: F(3,53)=2.080 p=0.1139). MW073 was administered at a concentration of 5 mg/Kg (i.p., daily, from day 70 until 100). The number of animals “n” is indicated on the graph.

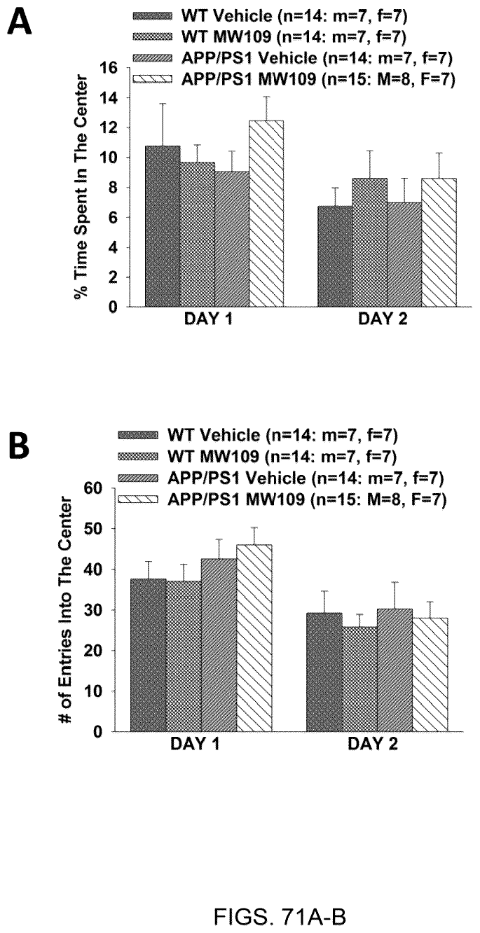

FIG. 71A-B show that MW109 has no effect on exploratory behavior in a prevention trial onto the APP/PS1 mouse model of amyloid elevation. Open field test shows a similar percentage of time spent in the center (day 1: F(3,53)=0.6323, p=0.5975; day 2: F(3, 53)=0.8753, p=0.4598) (A) and number of entries into the center among all conditions (day 1: F(3, 53)=0.6694, p=0.5746; day 2: F(3,53)=0.1105, p=0.9536) (B), indicating no differences in exploratory behavior. MW109 was administered at a concentration of 5 mg/Kg (i.p., daily, from day 70 until 100). The number of animals “n” is indicated on the graph.

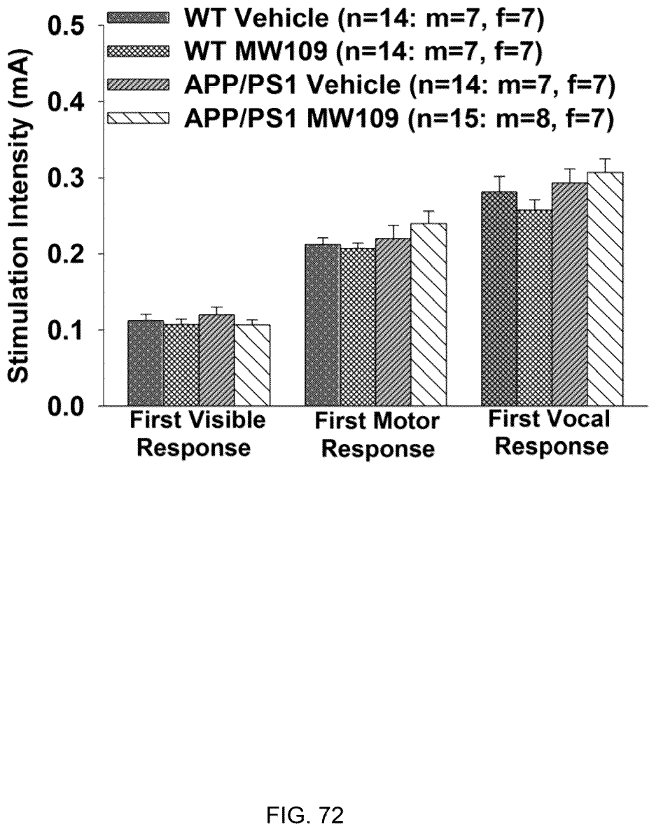

FIG. 72 shows that MW109 has no effect on animal capability of perceiving the electric shock in a prevention trial onto the APP/PS1 mouse model of amyloid elevation. No difference is detected among the groups during assessment of the sensory threshold. One-way ANOVA among all: for visible response F(3,53)=0.7578 p=0.5228; for motor response F(3,53)=1,261, p=0.2972 and for audible response F(3,53)=1.398, p=0.2535. MW109 was administered at a concentration of 5 mg/Kg (i.p., daily, from day 70 until 100). The number of animals “n” is indicated on the graph.

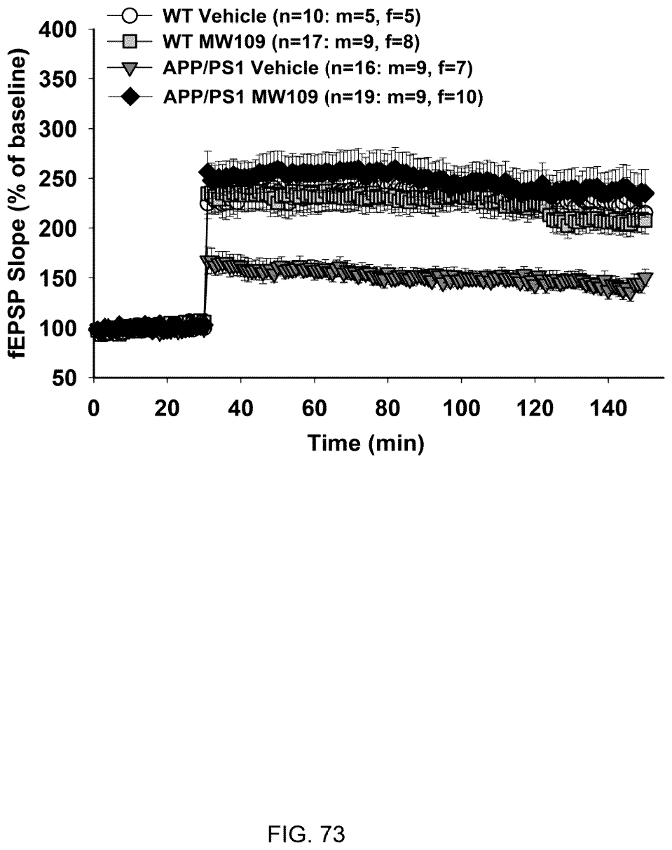

FIG. 73 shows the efficacy of MW109 onto the APP/PS1 mouse model of amyloid elevation in a prevention trial using LTP as a test of synaptic plasticity. MW109 (5 mg/Kg, i.p., daily, from day 70 until 110) restores animal capability of undergoing potentiation in APP/PS1 mice. MW109 alone in control WT mice does not affect potentiation. 2-way ANOVA F(3,58)=8.751, p<0.0001 ANOVA for repeated measures between groups: F(1,25)=0.03530, p=0.8525 WT vehicle vs WT+MW109; F(1,24)=26.30, p<0.0001 WT vehicle vs. APP/PS1 vehicle; F(1,33)=20.11, p<0.0001 APP/PS1 vehicle vs. APP/PS1+MW109; F(1,34)=0.9263, p=0.3426 WT+MW109 vs. APP/PS1+MW109; The number of slices “n” is indicated on the graph.

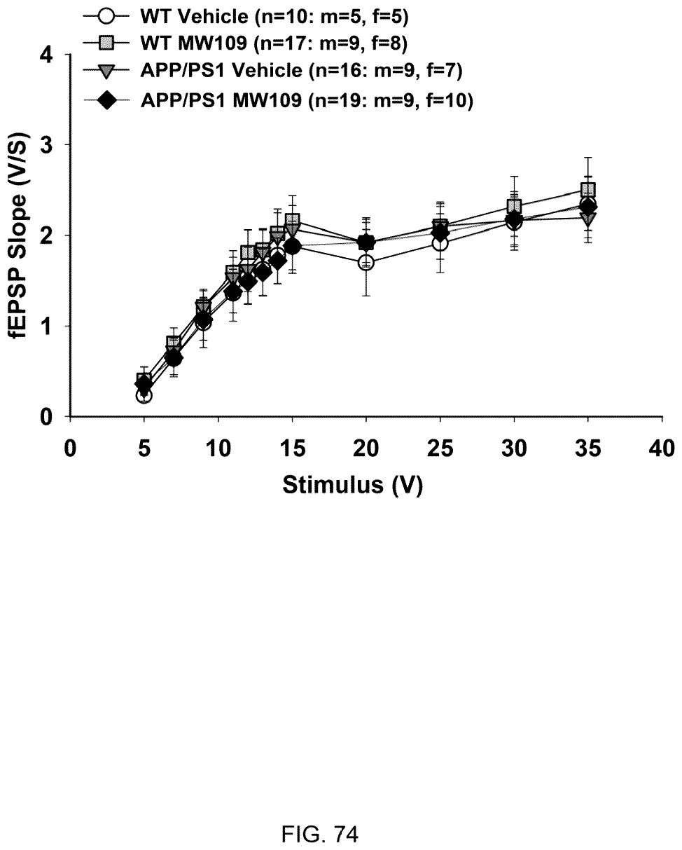

FIG. 74 shows that MW109 has no effect on basal neurotransmission in the APP/PS1 mouse model of amyloid elevation during a prevention trial. MW109 (5 mg/Kg, i.p., daily, from day 70 until 110) does not alter the input/output relationship in a test of basal synaptic transmission. 2-Way-ANOVA: F(3,58)=0.1556, p=0.9257. The number of slices “n” is indicated on the graph.

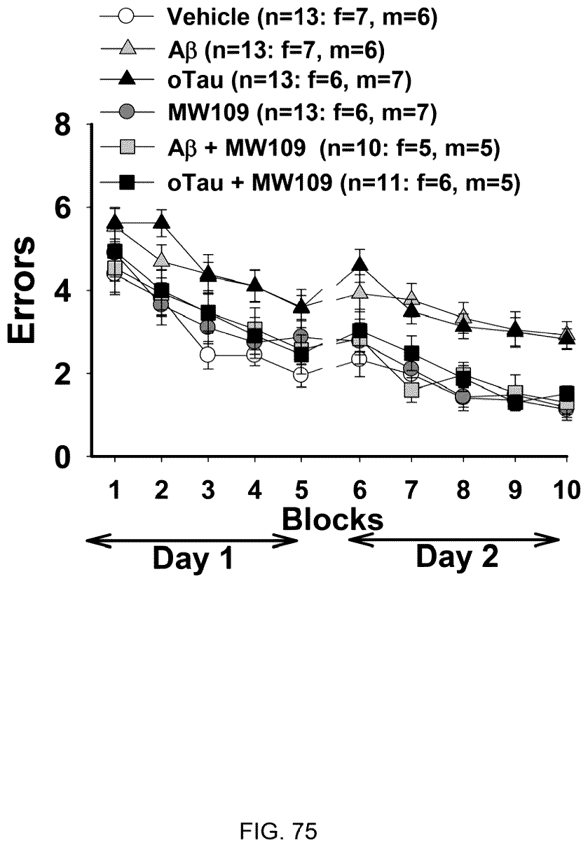

FIG. 75 shows efficacy of MW109 in Alzheimer relevant mouse models characterized by Aβ and tau oligomer elevation through their exogenous application using a test of spatial memory, the radial arm water maze (RAWM). MW109 (5 mg/Kg, i.p., 1 injection 30 min before the first trial and the seventh trial both on day 1 and 2) rescues the spatial memory defect in C57Bl6 mice infused with 200 nM Aβ- or 500 nM tau-oligomers, while MW109 alone in vehicle infused C57Bl6 mice does not affect performance. RAWM: 2 way-ANOVA for repeated measures among all groups (day 2): F(5,67)=12.88, p<0.0001. One-way ANOVA for block 10: F(5,67)=10.19, p<0.0001; One-way ANOVA for block 9: F(5,67)=7.026, p<0.0001 One-way ANOVA for block 8: F(5,67)=7.715, p<0.0001 One-way ANOVA for block 7: F(5,67)=6.049, p=0.0001. The number of animals “n” is indicated on the graph.

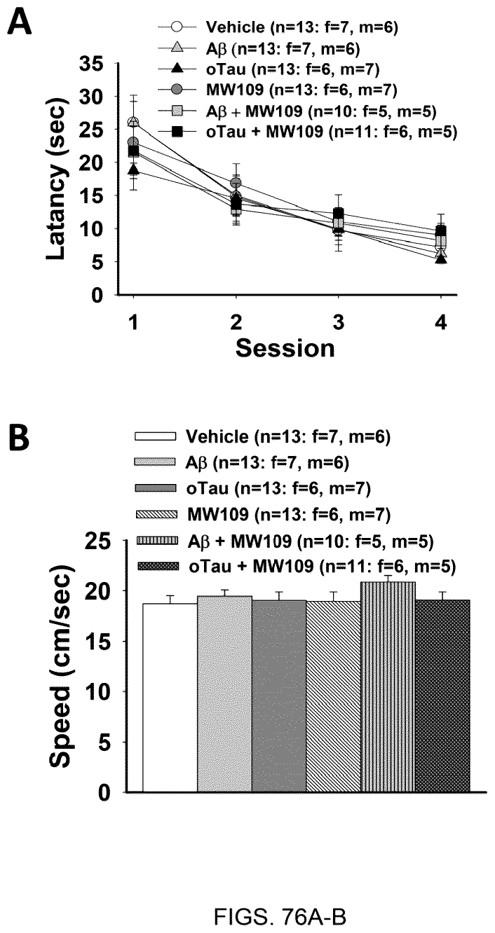

FIG. 76A-B illustrate that MW109 has no effect on vision, motility, or motivation in C57Bl6 mice infused with Tau or Aβ oligomers. Testing with the visible platform task does not reveal any difference in time to reach the visible platform (2-way ANOVA: F(5,67)=0.2532, p=0.9368) (A) and average speed (1-way ANOVA: F(5,67)=0.8191, p=0.5404) (B). MW109 was administered at a concentration of 5 mg/Kg (i.p., 30 min prior the 1st session over 2 days). The number of animals “n” is indicated on the graph.

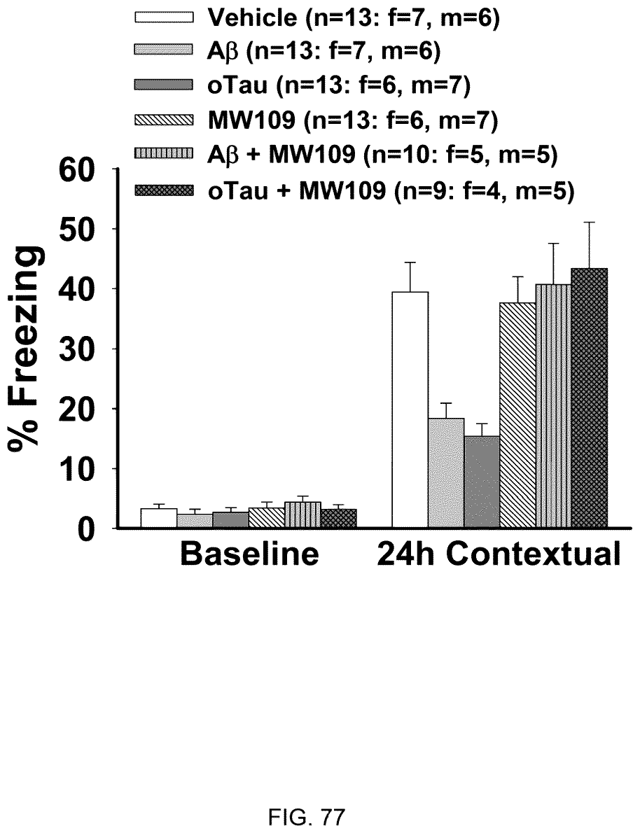

FIG. 77 shows efficacy of MW109 in AD relevant mouse models characterized by Aβ and tau oligomer elevation using a test of associative memory, the contextual fear conditioning (FC). MW109 (5 mg/Kg, i.p., 1 injection 30 min prior to the electric shock) protects C57Bl6 mice infused with 200 nM Aβ oligomers and 500 nM tau oligomers (oTau) against the impairment of associative memory, while MW109 alone in vehicle infused C57Bl6 mice does not affect performance. FC: ANOVA F(5,65)=6.924, p<0.0001; Bonferroni p=0.022 veh vs. Aβ, p=0.0049 veh vs. Tau, p=0251 Aβ vs. Aβ+MW109, p=0025 Tau vs. Tau+MW109. The number of animals “n” is indicated on the graph.

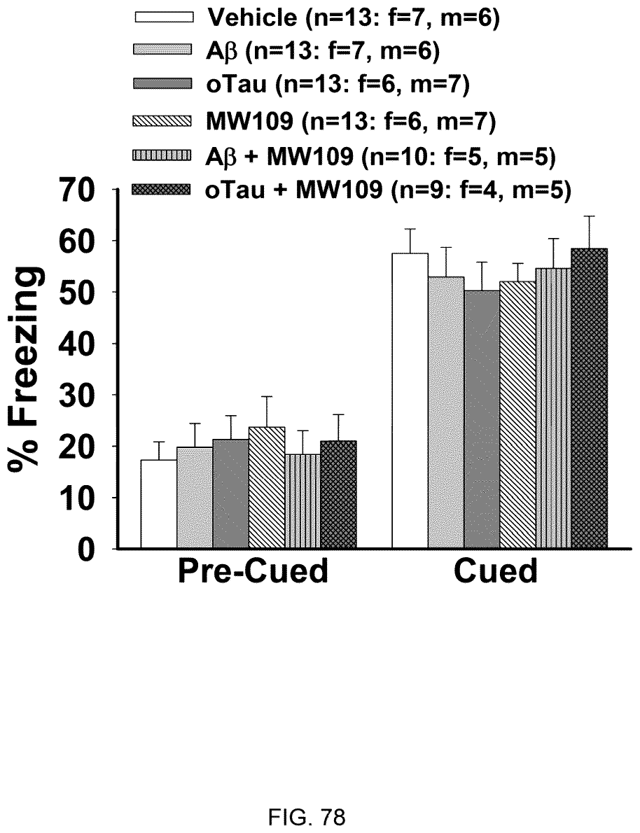

FIG. 78 shows that MW109 has no effect on cued amygdala dependent memory in C57Bl6 mice infused with Tau or Aβ oligomers. Freezing responses during the auditory cued conditioning are not significantly different among the groups (1-way ANOVA: F(5,65)=0.3559, p=0.8766). MW109 was administered at a concentration of 5 mg/Kg (i.p., 1 injection 30 min prior to the sound). The number of animals “n” is indicated on the graph.

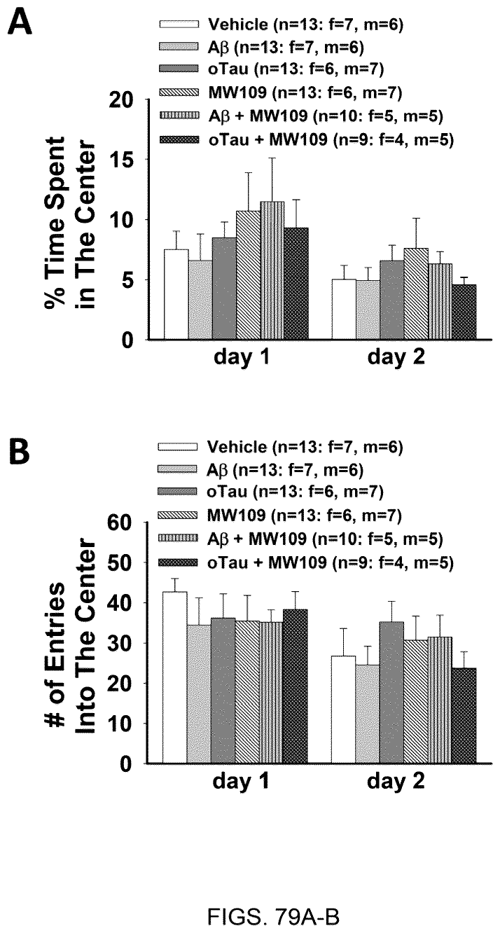

FIG. 79A-B illustrate that MW109 has no effect on exploratory behavior in C57Bl6 mice infused with Tau or Aβ oligomers. Open field test shows a similar percentage of time spent in the center (A) (one-way ANOVA day 1: F(5,65)=0.5921, p=0.7060; day 2: F(5, 65)=0.6195, p=0.6854) and (B) number of entries into the center among all conditions (one-way ANOVA day 1: F(5, 65)=0.3317, p=0.8920; day 2: F(5,65)=0.6280, p=0.6790), indicating no differences in exploratory behavior. MW109 was administered at a concentration of 5 mg/Kg (i.p., 1 injection 30 min prior to the test, both on day 1 and 2). The number of animals “n” is indicated on the graph.

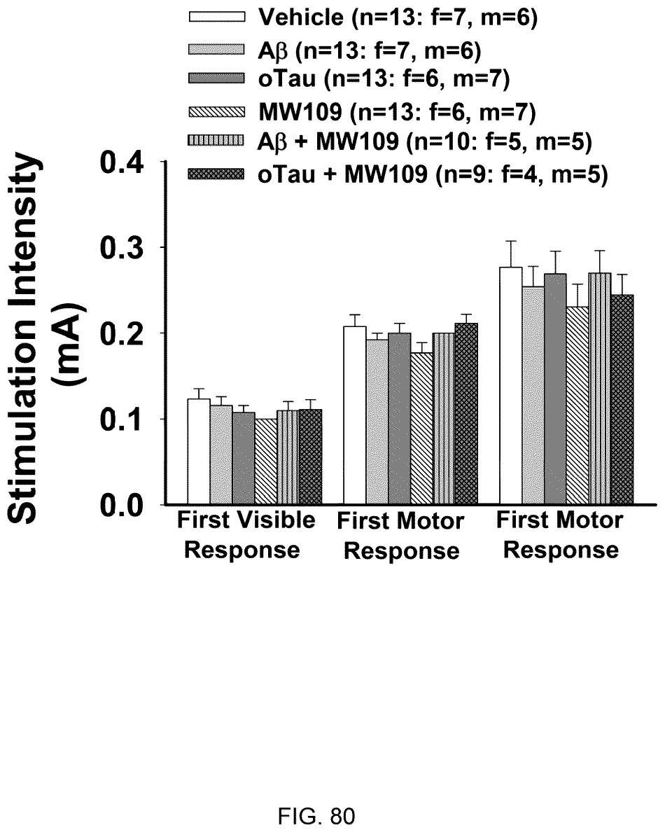

FIG. 80 shows that MW109 has no effect on animal capability of perceiving the electric shock in C57Bl6 mice infused with Tau or Aβ oligomers. No difference is detected among the groups during assessment of the sensory threshold after administration of MW109 (5 mg/Kg, i.p, 1 injection 30 min prior to the test). One-way ANOVA among all: for visible response F(5,65)=0.7488, p=0.5889; for motor response F(5,65)=1.295, p=0.2769 and for audible response F(5, 65)=0.4517, p=0.8106. The number of animals “n” is indicated on the graph.

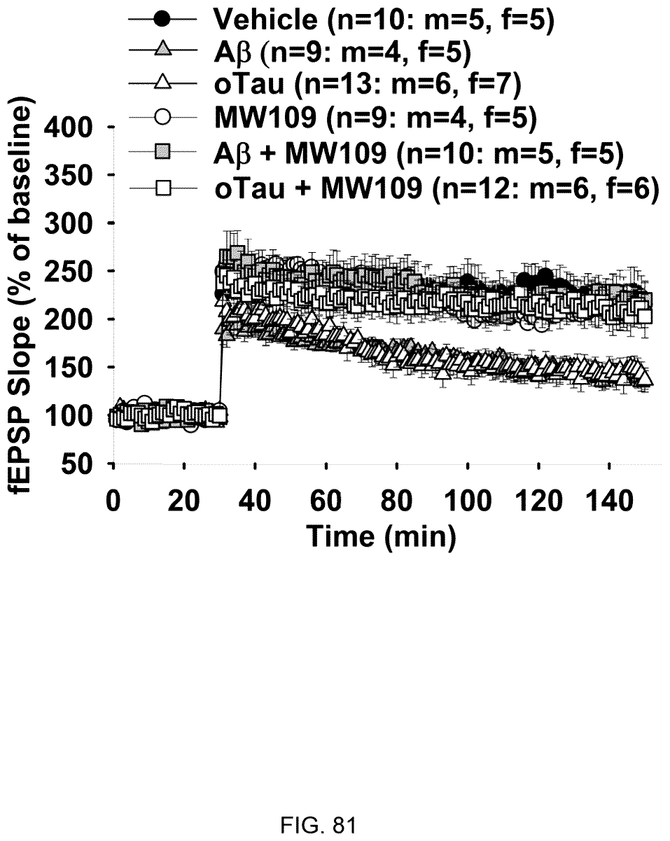

FIG. 81 shows efficacy of MW109 in AD relevant mouse models characterized by Aβ and tau oligomer elevation using long-term potentiation (LTP), as a test of synaptic plasticity. Perfusion with MW109 (10 M) rescues LTP defect in hippocampal slices from C57Bl6 mice treated with 200 nM Aβ- or 50 nM tau-oligomers. MW109 alone does not affect potentiation. ANOVA for repeated measures between groups: F(1,21)=9.909, p=0.0049 vehicle vs. Tau; F(1,17)=10.63, p=0.0046 vehicle vs. Aβ; F(1,23)=8.113, p=0.0091 Tau vs. Tau+MW109; F(1,17)=15.85, p=0.001 Aβ vs. Aβ+MW109; (F(1, 17)=0.02012, p=0.8889 vehicle vs. MW109. The number of slices “n” is indicated on the graph.

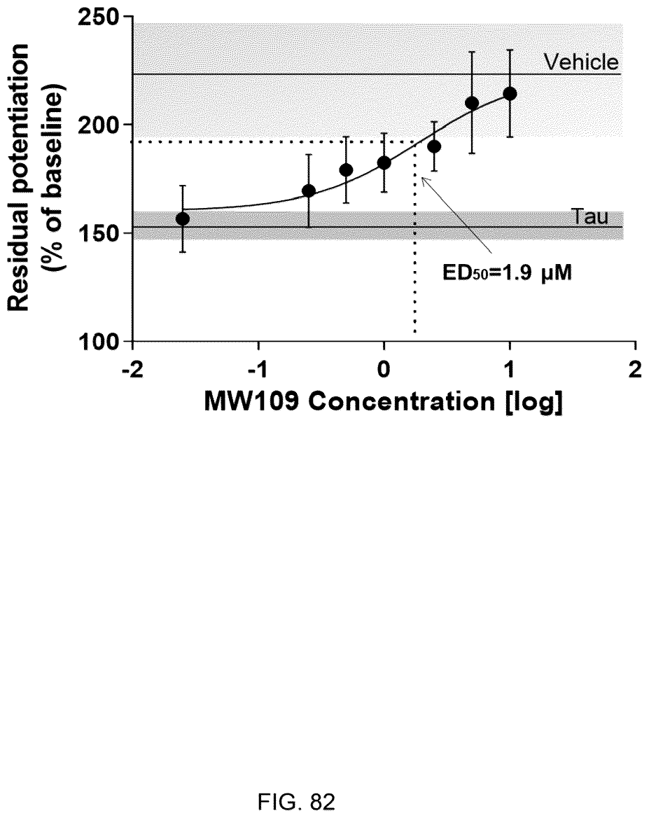

FIG. 82 shows a dose-response curve with ED50 for the beneficial effect of MW109 onto the long-term potentiation (LTP) defect in hippocampal slices perfused with oligomeric tau. The values of ED50 is indicated in the graph. Slices derived from the hippocampus of C57Bl6 mice were perfused with increasing concentrations of MW109. The amount of potentiation during the last 5 min after 120 minutes recording from the Theta-burst is plotted on the Y axes. Slices were co-perfused with 50 nM oligomeric tau and different concentrations of MW109 for 20 min prior to the theta-burst. For these experiments 9-25 slices per group were tested. The shaded area at the bottom corresponds to the average potentiation (continuous line) and the standard error range in tau-perfused slices. The shaded area at the top corresponds to the average potentiation (continuous line) and the standard error range in vehicle-perfused slices.

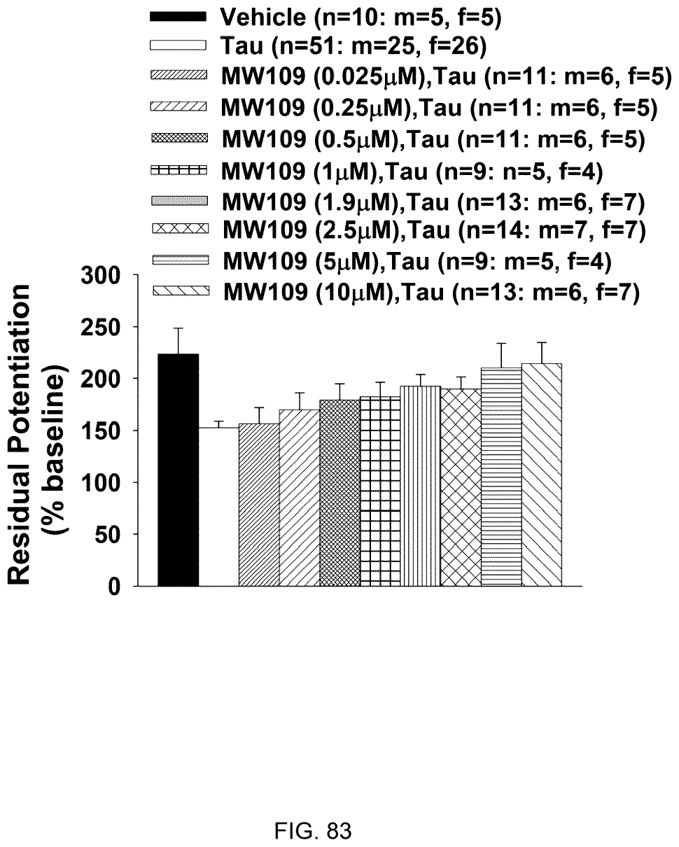

FIG. 83 shows a dose-response curve for the beneficial effect of MW109 onto the long-term potentiation (LTP) defect in hippocampal slices perfused with oligomeric tau. Slices derived from the hippocampus of C57Bl6 mice were perfused with increasing concentrations of MW109. The amount of potentiation during the last 5 min after 120 minutes recording from the theta-burst is plotted on the Y axes. Slices were co-perfused with of 50 nM oligomeric tau and different concentration of MW109 for 20 min prior to the theta-burst. The number of slices and sex of the animals providing the slices is shown on the figure. m (males); f (females) in this and the following graphs.

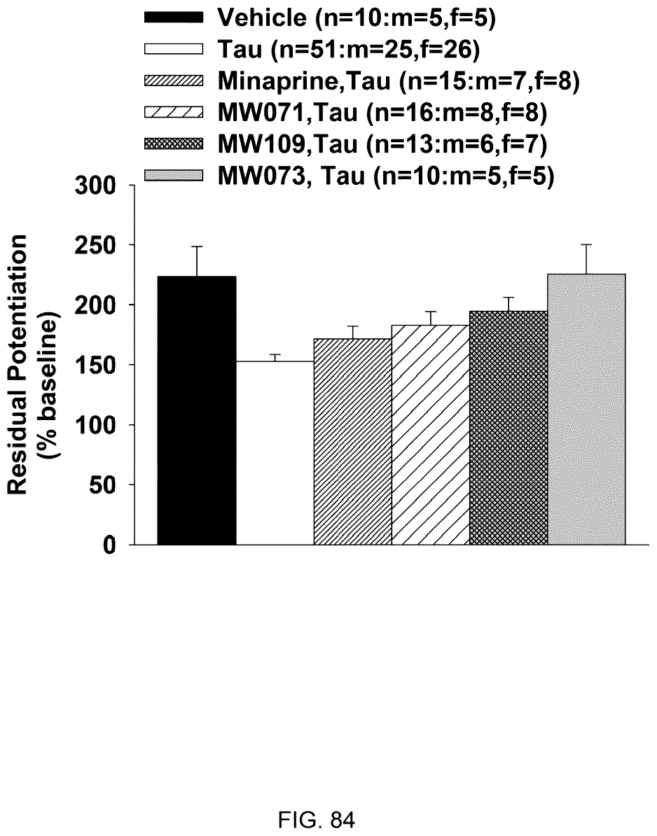

FIG. 84 shows a phenotypical screening of different small molecules (minaprine, MW071, MW073, and MW109) based on the ability to rescue the detrimental effect of 50 nM oligomeric tau onto long-term potentiation (LTP) in slices from the hippocampus of C57Bl6 mice when co-administered at 1.9 μM for 20 min (the ED50 for MW109). Overall 1-way ANOVA: F(5, 108)=6.626; P<0.0001. Bonferroni's multiple comparisons: Vehicle vs. Tau: p=0.0004, Tau vs. Tau+Minaprine: p>0.999, Tau vs. MW071: p=0.0859, Tau vs. Tau+MW109: p=0.0402, Tau vs. Tau+MW073: p=0.0002. MW073 and MW071 showed efficacy in tests of memory and its synaptic surrogate, LTP, in various animal models of Alzheimer's disease. MW071 showed antagonist activity only for the 5-HT2b receptor in large scale activity screens of known G-protein coupled receptors (GPCRs). Importantly, there was no 5-HT2b receptor agonist activity, which is major safety risk factor. Confirmation of the positive hit for 5-HT_{2b} receptor antagonist activity was done by determination of MW071's dose-dependent antagonist activity, which revealed an IC₅₀ of about 40 nM. Additional pharmacological screens demonstrated that MW071 was not a MAO inhibitor or modulator of diverse transporters. Taken in its entirety, the pharmacological screening demonstrated that MW071 is a highly selective molecular probe with potential for exploring the role of 5HT2bR antagonism in synaptic and cognitive dysfunction. The functional utility of MW071 was explored by synaptic function screens ex vivo and behavioral functions in mouse models. However, continued pharmacological analyses revealed some inherent limitations of MW071, such as substrate status for Pgp transporter. Developing other compounds with acceptable properties was difficult due to the convergence of multiple parameters. Unexpectedly, MW073 met those parameters. MW073 employs a 2-naphthyl ring in place of the phenyl found in minaprine or MW071, and methylation of the secondary amine in the piperazine ring of MW071. MW073 stays within the multi-property features associated with blood:brain barrier permeation, avoids CYP2D6 and Pgp substrate status, and retains human liver microsome stability. Importantly, MW073 retains the remarkable target selectivity of functions of MW071, with an IC₅₀ of 66 nM. Overall, MW073 is small molecule that offers an improved approach to treating behavior and cognition needs that are common across multiple neurodegenerative diseases.

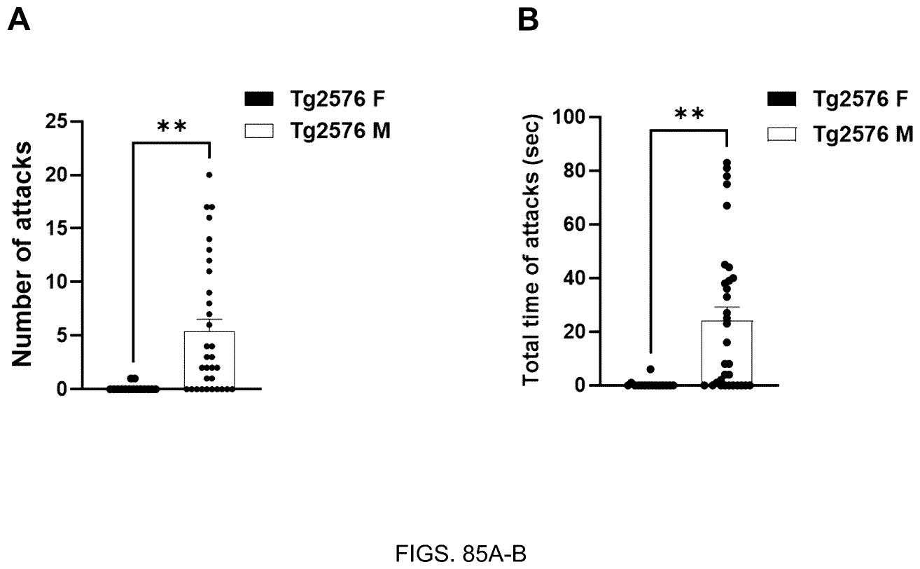

Testing was conducted to determine whether there were sex differences in aggressive behavior among Tg2576 mice. FIG. 85A is a bar graph showing that no Tg2576 resident females attacked an intruder female during the 10 minutes resident-intruder test session. In contrast, 21 out of 32 Tg2576 resident males attacked the intruder male. Number of attacks (FIG. 85A) and total attack time (FIG. 85B) were dramatically increased in Tg2576 males compared to Tg2576 female mice. For this reason, it was decided to perform the following experiments in which MW073 was administered to the animal with Tg2576 male residents.

FIGS. 85A-B show that Tg2576 males are more aggressive than Tg2576 females. Bar graphs representing rate scores related to aggressive behavior in Tg2576 resident males and Tg2576 resident females. The number of attacks (FIG. 85A), as well as the total attack time (FIG. 85B), of male Tg2576 animals was dramatically higher than in females Tg2576 mice (number of attacks: Tg2576 females=0, Tg2576 males=7.813±2.203, t-test for this and all the other graphs: p=0.0016; total attack time: Tg2576 females=0 sec, Tg2576 males=37.00±0.915 sec, p<0.0001). These experiments were performed on 10 Tg2576 female mice and 16 Tg2576 male mice. Standard errors and results from individual experiments are shown within the column bars in this and the following figures. ns=not significant for this and the following figures.

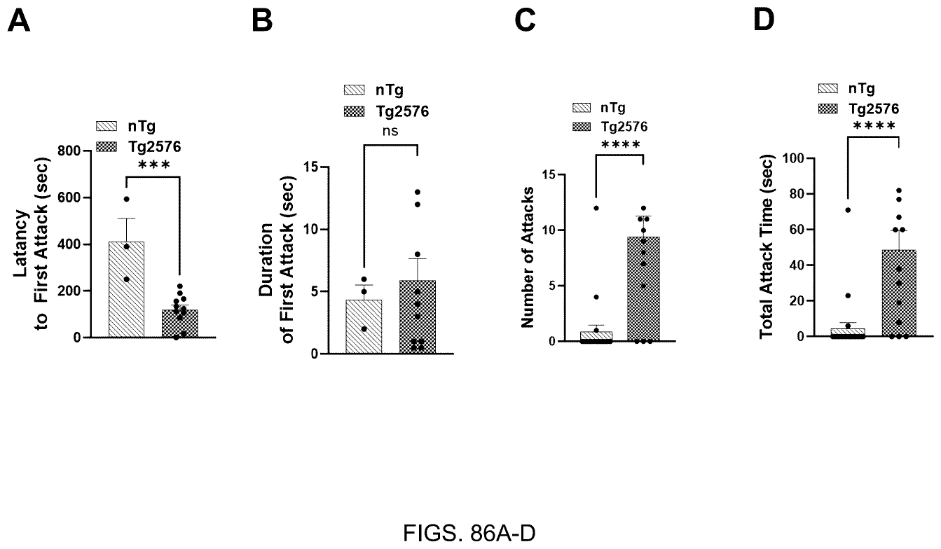

Aggressive behavior was tested comparing Tg2576 and non-transgenic (nTg) littermate males. The data revealed that just 3 nTgs out of 20 resident males attacked an intruder male during the 10 minutes resident-intruder test sessions. In contrast, 11 out of 14 Tg2576 residents attacked the intruder male (FIG. 86A-D). When the intruder was inserted into the cage, resident Tg2576 mice started the first attack after ˜2 minutes revealing a clear aggressive behavior. However, when the intruder was inserted into the cage, the resident nTg mouse started the first attack after ˜7 min (FIG. 86A). It was found that there was a slight increase in the duration of the first attack, but the increase was not statistically significant (FIG. 86B). Number of attacks (FIG. 86C) and total attack time (FIG. 86D) were dramatically increased in Tg2576 compared to nTg mice.

FIGS. 86A-D show that Tg2576 resident males are more aggressive than nTg resident males. Bar graphs representing rate scores related to aggressive behavior in Tg2576 and non-transgenic (nTg) resident males. FIG. 86A shows that nTg residents displayed longer latency to the first attack compared to Tg2576 mice (nTg=411.3±99.87 sec, Tg=119.5±20.27 sec, p=0.0004). FIG. 86B shows that compared to nTg residents, Tg2576 residents showed slightly higher but not statistically significant duration of the first attack (nTg=4.33±1.20 sec, Tg=5.909±1.74 sec, p=0.6590). FIGS. 86C-D show that the number of attacks, as well as the total attack time, respectively, of Tg animals was dramatically higher compared to nTg mice (number of attacks: nTg=0.850±0.62, Tg=9.429±1.86, p<0.0001; total attack time: nTg=5.00±9.46 sec, Tg=48.43±11.04 sec, p=0.0002). These experiments were performed on 20 nTg mice and 14 Tg2576 male mice. The average age of nTg mice was 224.2±40.218, whereas the average age of the Tg2576 was 181±13.284 (p=0.3897).

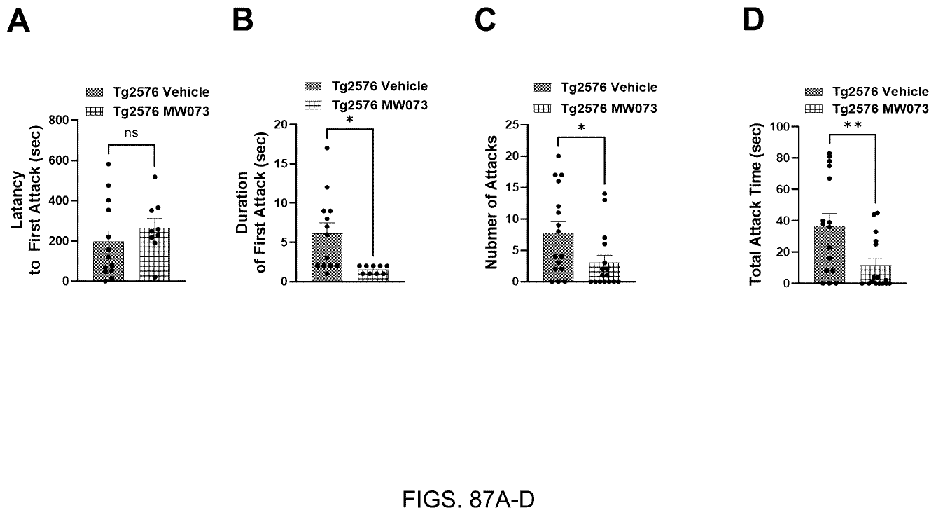

Next, testing was conducted to determine whether MW073 was capable of reducing the aggressive behavior of Tg2576 male mice. FIGS. 87A-D show that aggressive behavior is reduced in Tg2576 mice treated with MW073 compared to Tg2576 mice treated with vehicle. Animals were treated with the 5HT2b receptor antagonist for 3 weeks (daily, i.p., 5 mg/kg) prior to performing the aggressivity test. The data revealed that Tg2576 resident males treated with the serotonin antagonist (n=16) showed less aggressivity than Tg2576 mice treated with vehicle (n=16) during the 10 minutes resident-intruder test sessions. Specifically, when the intruder was inserted into the cage, resident Tg2576 mice treated with vehicle started the first attack after ˜3 min. By contrast, when the intruder was inserted into the cage, resident Tg2576 mice treated with MW073 started the first attack after ˜4.45 min. Despite the delay in the first attack for Tg mice treated with MW073, the difference was not statistically significant (FIG. 87A). Most importantly, the duration of the first attack in Tg mice treated with MW073 was reduced (FIG. 87B). Moreover, the number of attacks and the time of total attacks was lower in Tg2576 treated with MW073 compared to Tg mice treated with vehicle (FIGS. 87C-D).

FIGS. 87A-D show that Tg2576 mice treated with MW073 showed amelioration of aggressive behavior. Bar graphs representing rate scores related to aggressive behavior in Tg2576 mice treated with vehicle and Tg2576 treated with MW073. FIG. 87A shows that Tg2576 resident males treated with MW073 displayed a slightly longer but not statistically different latency to the first attack compared to Tg2576 treated with vehicle (vehicle=198.2±53.44 sec; MW073=266.8±45.75 sec, p=0.371 ns.). FIG. 87B shows that Tg2576 resident males treated with MW073 showed a decrease in the duration of the first attack (vehicle=6.154±1.33 sec; MW073=1.556±0.17, p=0.01). FIGS. 87C-D show that the number of attacks, as well as the total attack time, respectively, of Tg2576 resident males treated with MW073 was reduced compared to Tg2576 treated with vehicle (number of attacks: vehicle=7.813±1.72, MW073=3.063±1.15, p=0.029; total attack time: vehicle=37.00±7.77, MW073=11.56±4.23, p=0.0074). These experiments were performed on 16 Tg mice either treated with vehicle or MW073. The average age of Tg2576 mice treated with vehicles was 294.56±34.550 days, whereas the average of mice treated with Mw073 was 280.81±32.954 days (p=0.7753).

The following groups of mice were used: a) C57BL/6 mice used like control in each experiment, and b) the Tg2576 mouse model of amyloid deposition. It overexpresses human mutant APP (isoform 695) containing the double mutation K670N, M671L (Swedish mutation) under the control of the hamster prion protein promoter. They are characterized by elevated levels of Aβ and ultimately amyloid plaques. Tg2576 mice derived from a mouse colony bred at Columbia University. Animals were maintained on a 12-hour light/12-hour dark cycle, in a temperature- and humidity-controlled room. Food and water were available ad libitum. Mice were allocated to a specific treatment and paradigm by a randomization procedure. Investigators who performed the experiments were blind in respect to genotype and treatment. At the time of weaning, the animals were genotyped using tail biopsies, followed by enzymatic digestion and polymerase chain reaction analysis.

MW073 compound for treatment in behavioral testing was diluted in 10% Propylene Glycol (Sigma-Aldrich P4347) MilliQ quality water and 0.1% formic acid to prepare a stock solution. At the time of the experiment, the compound was diluted in sterile saline and administered by i.p. injection at concentration of 5 mg/kg.

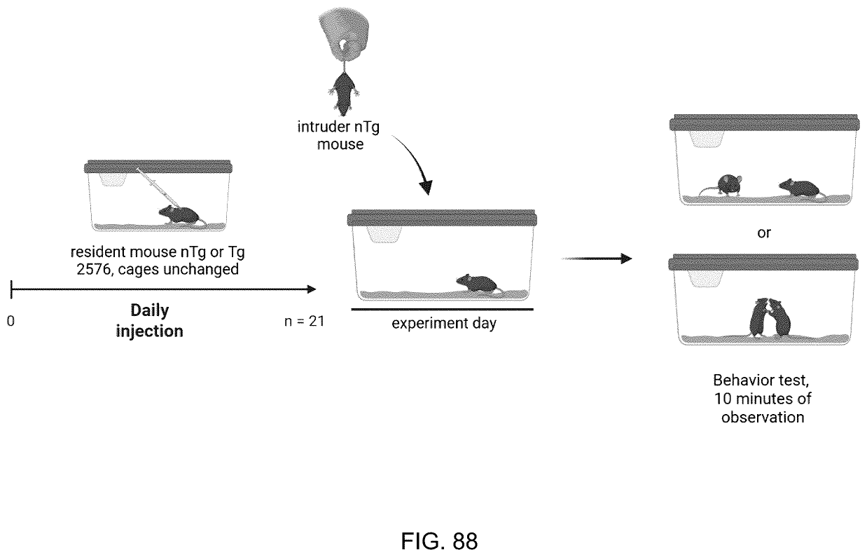

This study aimed to elucidate the effect of a 5HT2b receptor antagonist on spontaneous social behavior mice (FIG. 88). Mice aggressive behavior was assessed by means of a combined isolation-induced and resident-intruder paradigm. For this purpose, mice were isolated for 3 weeks. The mice were single housed in their standard cages and left undisturbed during the entire isolation period. Meanwhile, no fresh bedding material was provided to ascertain that the area becomes their own territory and to evoke aggressive behavior upon intrusion by another mouse of the same sex. After 3 weeks of isolation, mice were allowed to adapt to the observation room in their home cage for at least 1 hour prior to testing. A group-housed male C57Bl6 mouse was introduced into the resident's home cage. Only the behavior of resident mice was analyzed. The second mouse was classified as an intruder. To distinguish the intruder from residence mouse, the intruder was marked with a black sign on the tail. The behavior was recorded for 10 minutes.

FIG. 88 provides a schematic showing the experimental design for the testing related to aggressiveness. Resident mice (either nTg or Tg) were housed individually for the 21-day injection period. On the test day, an intruder nTg mouse was introduced into the resident's cage, and the social interactions between the animals were observed and recorded for 10 minutes immediately following the introduction.

During a 10-min observation period, the observer, who was blind to the mouse's genotype, evaluated animal aggressivity. As an index of social behavior and aggressivity, pouncing/chasing behaviors (chasing, attacking and escalated fighting) of the mouse were considered as discriminating against the approaching, facial/body sniffing, ano-genital sniffing from resident mice. The observer recognized defensive behavior such as avoiding, fleeing and defensive upright posture. The number/severity of physical encounters was closely monitored, and the mice were separated if any encounter was severe enough to potentially cause injury. The number of encounters and latency encounters were scored using a stopwatch and every test was recorded. (Noldus Information Technology, Wageningen, The Netherlands).

Investigators who performed the experiments were blinded with respect to treatment and genotype. Behavioral data were quantified as the total duration of each behavior exhibited during the 10-minute observation period. Comparisons of a single variable between Tg and nTg mice, as well as between vehicle- and MW073-treated Tg mice, were performed using two-tailed Student's t-tests in GraphPad Prism 10.

### Western Blotting on Human Sample

Human brain tissue was homogenized in lysis buffer (62.5 mM Tris-HCl pH 6.8, 3% SDS, Halt™ Protease and Phosphatase Inhibitor Cocktail (100×) and incubated at 4° C. for 60 min, then sonicated before centrifugation at 13,000 rpm for 15 min. 5-HT-2B antibodies (Abcam 227722 1:1000, 5% milk in TRIS-buffered saline (TBS) 1×), and GAPDH antibodies (1:5000, CB100 5% milk in TBS) were from MilliporeSigma (St. Louis, MO, USA). For quantitative immunoblot analysis, equal amounts of proteins were loaded into each lane. Blots were re-probed with corresponding pan-antibodies and antibodies for tubulin or GAPDH to confirm equal loading. For quantification, we used a signal in the linear range. Immunoblot data were quantified by measuring the band intensity using imaging software (NIH ImageJ).

### Animals

C57BL/6J were obtained from breeding colonies kept in the animal facility of Columbia University. They were 3-4 months of age. Both sexes were used. All mice were maintained on a 12 hr light/dark cycle (lights on at 6:00 AM) in temperature and humidity-controlled rooms; food and water were available ad libitum. APP/PS1 mice were obtained from breeding colonies kept in the animal facility of Columbia University. APP/PS1 mice are heterozygous double transgenic mice expressing both human APP (K670N:M671L) and human PS1 (M146L) (line 6.2). They were obtained by crossing heterozygous APP with PS1 animals. hTau/Mapt-KO mice were obtained by crossing hTau mice in a murine tau-hemyzygous background to generate hTau/Mapt-KO mice and siblings. The hTau animals express WT, full-length human tau (2N4R) driven by the prion promoter. At weaning, the animals were genotyped from tail biopsies by means of an appropriate digest and polymerase chain reaction.

Aβ oligomers

Human Aβ₄₂ oligomerization was obtained as follows: a protein film was prepared by dissolving Aβ₄₂ lyophilized powder (AnaSpec, CA, USA) in 1,1,1,3,3,3-Hexafluoro-2-Propanol (HFIP) and subsequent incubation for 2 hrs at room temperature to allow complete monomerization. The Aβ film was dissolved in dimethylsulfoxide (DMSO), sonicated for 15 min, aliquoted, and stored at −20° C. To oligomerize the peptide, phosphate buffered saline (PBS) was added to an aliquot of DMSO-Aβ to obtain a 5 mM solution that was incubated for 12 hr at 4° C. This oligomerized Aβ solution was then diluted to the final concentration of 200 nM in artificial cerebrospinal fluid (ACSF) composed as following: 124.0 NaCl, 4.4 KCl, 1.0 Na₂HPO₄, 25.0 NaHCO₃, 2.0 CaCl₂, 2.0 MgCl₂ in mM.

### Tau Oligomers

Tau oligomers were obtained follows: the tau 4R/2N construct was prepared in expression vector pET29a (Bioclone) in the bacterial strain BL21 (DE3) for protein expression. For oligomerization, tau was transferred to protein concentrators and buffer exchanged with oligomerization buffer following incubation with 1 mM H₂O₂ at room temperature for 20 h for introducing disulfide bonds. Tau protein concentration was determined from the absorption at 280 nm with an extinction coefficient of 7450 cm-1M-1 and oligomers were visualized through Western blots.

### Electrophysiological Recordings

Mice were sacrificed through cervical dislocation and hippocampus was removed immediately after decapitation. Transverse hippocampal slices (400 μm thickness) were cut on a tissue chopper and transferred to the recording chamber where the physiological conditions in the brain were maintained by perfusion of ACSF. Slices were allowed to recover for at least 90 min before commencing the extracellular field recordings. A bipolar tungsten electrode and a glass electrode filled with ACSF were placed in the Schaeffer collateral fibers and the CA1 Stratum radiatum, respectively. After establishing the input-output relationship, a 30 min stable baseline was recorded, and LTP was induced using a theta-burst stimulation (consisting of 4 pulses at 100 Hz, with bursts repeated at 5 Hz and each tetanus including 3 ten-burst trains separated by 15 sec). LTP was measured as field excitatory postsynaptic potential (fEPSP) slope expressed as percentage of the baseline and the results were represented as mean±SEM.

### Behavioral Studies

Treatment for behavioral test: MW071 and MW109 were diluted in sterile saline under sterile conditions and administered by i.p. injection. MW073 was diluted in 10% Propylene Glycolin MilliQ quality water and 0.1% formic acid to prepare a stock solution. At the time of the experiment, the compound was diluted in sterile saline and administered by gavage.

Stereotaxic surgery and infusion of Aβ₄₂ and Tau: implant of the cannulas onto the hippocampi was performed as follows: the coordinates were 2.46 mm posteriorly and 1.5 mm laterally from Bregma to a depth of 1.30 mm. After 6-9 days of recovery, awake mice were restrained and gently infused with 200 nM Aβ into dorsal hippocampi bilaterally or 500 nM tau.

2 Day RAWM and visible platform task: RAWM was performed as follows: mice were trained in 15 trials to identify the platform location in a goal arm by alternating between a visible and a hidden platform from trial 1 to 12, and by finding a hidden platform in the last three trials. During the second day, the same procedure was repeated by using only the hidden platform from trial 1 to 15. An entrance into an arm with no platform, or failure to select an arm after 10 see was counted as an error and the mouse was gently pulled back to the start arm. The goal arm did not change among trials, with a different starting arm on successive trials. Data were analysed and displayed as averages of blocks of 3 trials. A visible platform test was performed to control for possible visual, motor and motivational deficits. This consisted in a two-day test, with two sessions/day (each consisting of three 1 min trials), in which the time taken to reach a visible platform (randomly positioned in a different place each time), and the speed to reach it, were recorded.

Fear conditioning: fear conditioning was performed as follows: during the first day, mice were placed in the conditioning chamber for 2 min before the onset of a discrete tone [conditioned stimulus (CS)](a sound that lasted 30 sec at 2800 Hz and 85 dB). In the last 2 sec of the CS, mice were given a foot shock [unconditioned stimulus (US)] of 0.80 mA for 2 sec through the bars of the floor. After the CS/US pairing, mice were left in the conditioning chamber for 30 sec and then returned to their home cages. Freezing behavior, defined as the absence of all movements except for those necessitated by breathing, was automatically scored. During the second day, we evaluated contextual fear learning. Mice were placed in the conditioning chamber and freezing was measured for five consecutive minutes. During the third day, cued fear learning was evaluated by placing mice in a novel context for 2 min (pre-CS test), after which they were exposed to the CS for 3 min (CS test), and freezing was measured.

Open field: the test has been used for assessing exploratory behavior and anxiety levels. Mice were placed in a novel open environment consisting of Plexiglass transparent walls, the arena was divided into sectors (periphery and center). Each mouse started the test in the center of the arena, and was permitted to freely explore the arena for 10 min in two consecutive days. Both percent time spent in the center and number of entries into the center were scored. Their activity was automatically recorded for 10 min on two consecutive days.

Sensory threshold assessment: the test was used for evaluating the animal perception of the shock. Animals were subjected to 1 sec foot shocks of increasing intensity from 0.1 to 0.6 mA at 0.1 mA increments every 30 sec. The foot shock intensity that elicited the first visible response (flinching), the second motor response (jumping), and the first audible response (vocalization) were noted.

### Statistical Analyses

For electrophysiological recordings results were analyzed by two-way analysis of variance (ANOVA) for repeated measures comparing traces after tetanic stimulation with treatment condition as main effect. For the behavioral tests, results were analyzed by either two-way ANOVA for repeated measures or one-way ANOVA with Bonferroni post-hoc planned comparisons. Behavioral experiments were designed in a balanced fashion in which the sex of mice was kept equal between the various groups, and for each condition mice were trained and tested in three to four separate sets of experiments in blind. Differences were considered significant at a p value less than 0.05. Results were expressed as Standard Error of the Mean (SEM).

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed is:

1. A compound of Formula (I): 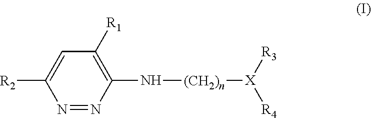
wherein R₁ is CN, C₁-C₆ alkyl, aryl, or heteroaryl; R₂ is phenyl, benzyl, naphthyl, quinolinyl, isoquinolinyl, or indolyl, wherein said phenyl, benzyl, naphthyl, quinolinyl, isoquinolinyl or indolyl is optionally independently substituted with at least one halogen; X is CH or N; n is 1 or 2; and R₃ and R₄ are independently H, C₁-C₆ alkyl, aryl or heteroaryl, provided that R₃ and R₄ are not both H, or R₃, R₄ together with the X to which they are attached form 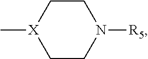
where R₅ is H, C₁-C₆ alkyl, aryl, or heteroaryl; or
a pharmaceutically acceptable salt thereof; or
a compound of Formula (II): 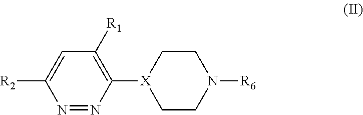
wherein
R₁ is CN, C₁-C₆ alkyl, aryl, or heteroaryl;
R₂ is benzyl, naphthyl, quinolinyl, isoquinolinyl, or indolyl, wherein said benzyl, naphthyl, quinolinyl, isoquinolinyl or indolyl is optionally independently substituted with at least one halogen;
X is CH or N; and
R₆ is H, C₁-C₆ alkyl, aryl, or heteroaryl;
or a pharmaceutically acceptable salt thereof;
with the proviso that when R₂ is phenyl, R₅ is not H;
or when R₁ and R₂ are both phenyl, neither one of R₃ or R₄ is pyrimidinyl; or when X is C, R₅ is not benzyl.

2. The compound of claim 1, wherein X is N.

3. The compound of claim 1, wherein
the compound is a compound of Formula (I), wherein
R₁ is CN, C₁-C₂ alkyl, phenyl, or pyridyl;
n is 2; and
R₃, R₄ together with the N to which they are attached form 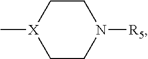
where R₅ is H, C₁-C₂ alkyl, aryl, or heteroaryl containing at least one 1 in an aryl ring;
or a pharmaceutically acceptable salt thereof.

4. The compound of claim 3, wherein
R₁ is methyl;
R₂ is phenyl, or naphthyl;
n is 2; and
R₃, R₄ together with the X to which they are attached form 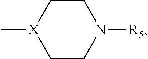
where R₅ is C₁-C₂ alkyl;
or a pharmaceutically acceptable salt thereof.

5. The compound of claim 1, wherein the compound is a compound of Formula (II),
wherein R₁ is CN, C₁-C₂ alkyl, phenyl, or pyridyl; and
R₆ is C₁-C₂ alkyl, or heteroaryl containing at least one N in an aryl ring;
or a pharmaceutically acceptable salt thereof.

6. The compound of claim 5, wherein R₁ is C₁-C₂ alkyl, phenyl, or pyridyl;
and R₆ is methyl, pyridinyl or pyrimidinyl;
or a pharmaceutically acceptable salt thereof.

7. The compound of claim 1, having the structure: 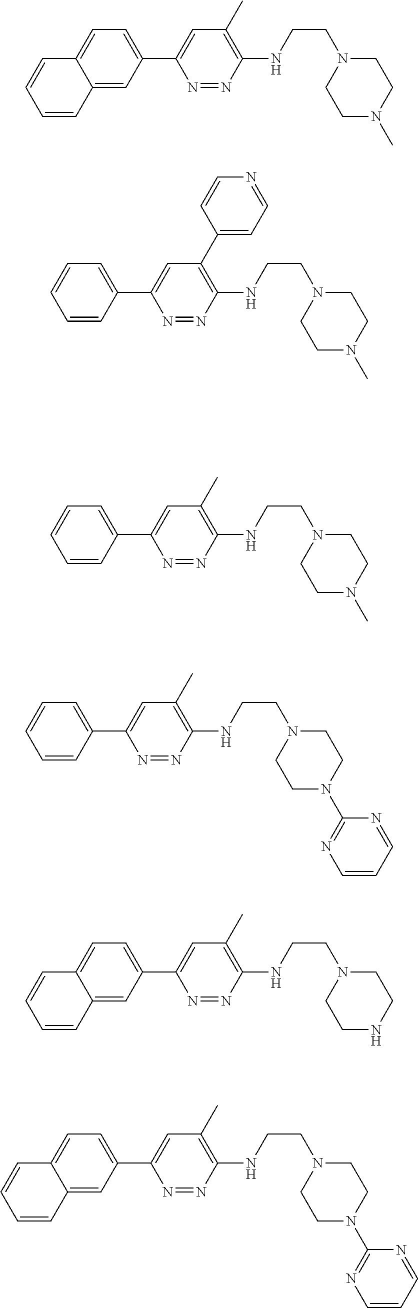 [SMILES: Cc1cc(-c2ccc3ccccc3c2)nnc1NCCN1CCN(C)CC1 ; CN1CCN(CCNc2nnc(-c3ccccc3)cc2-c2ccncc2)CC1 ; Cc1cc(-c2ccccc2)nnc1NCCN1CCN(C)CC1 ; Cc1cc(-c2ccccc2)nnc1NCCN1CCN(c2ncccn2)CC1 ; Cc1cc(-c2ccc3ccccc3c2)nnc1NCCN1CCNCC1 ; Cc1cc(-c2ccc3ccccc3c2)nnc1NCCN1CCN(c2ncccn2)CC1] 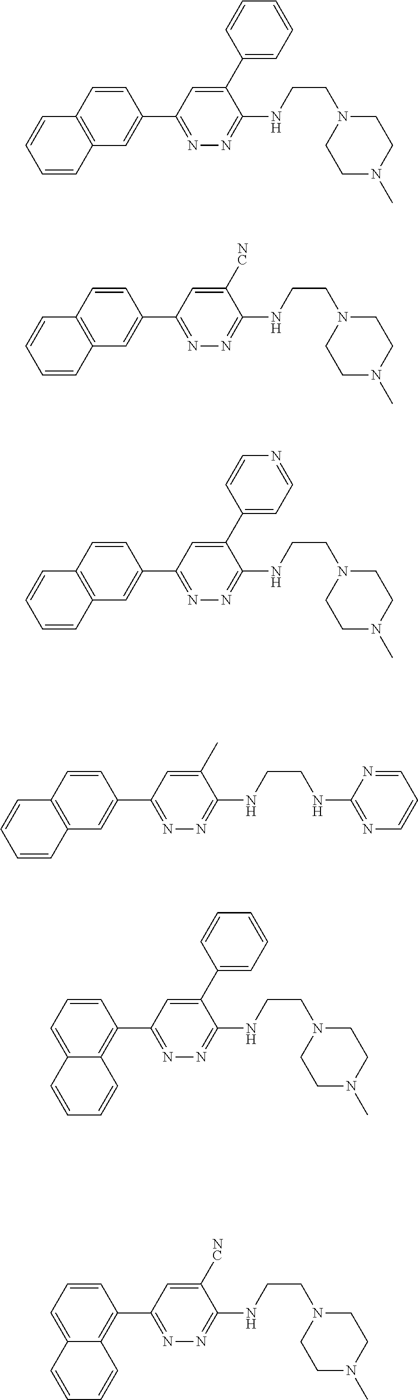 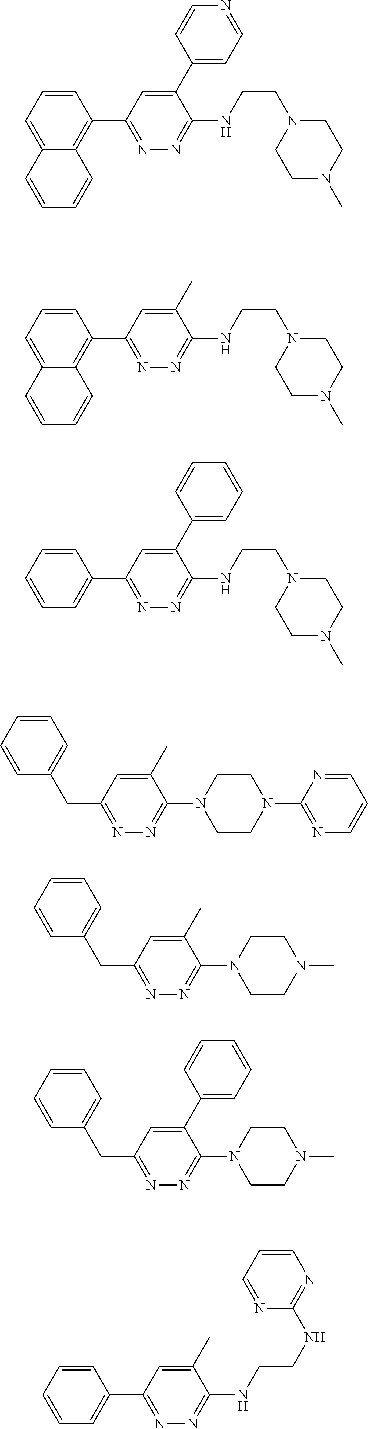 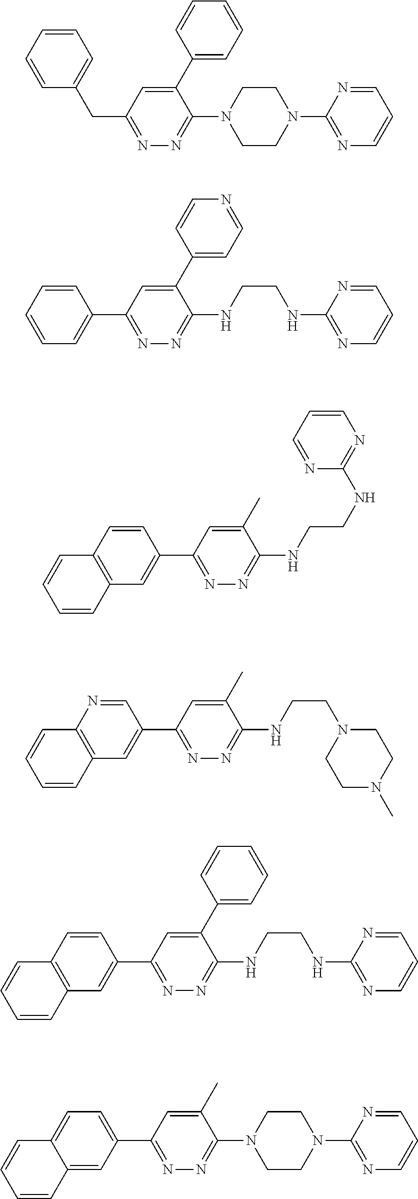 [SMILES: c1ccc(Cc2cc(-c3ccccc3)c(N3CCN(c4ncccn4)CC3)nn2)cc1 ; c1ccc(-c2cc(-c3ccncc3)c(NCCNc3ncccn3)nn2)cc1 ; Cc1cc(-c2ccc3ccccc3c2)nnc1NCCNc1ncccn1 ; Cc1cc(-c2cnc3ccccc3c2)nnc1NCCN1CCN(C)CC1 ; c1ccc(-c2cc(-c3ccc4ccccc4c3)nnc2NCCNc2ncccn2)cc1 ; Cc1cc(-c2ccc3ccccc3c2)nnc1N1CCN(c2ncccn2)CC1] 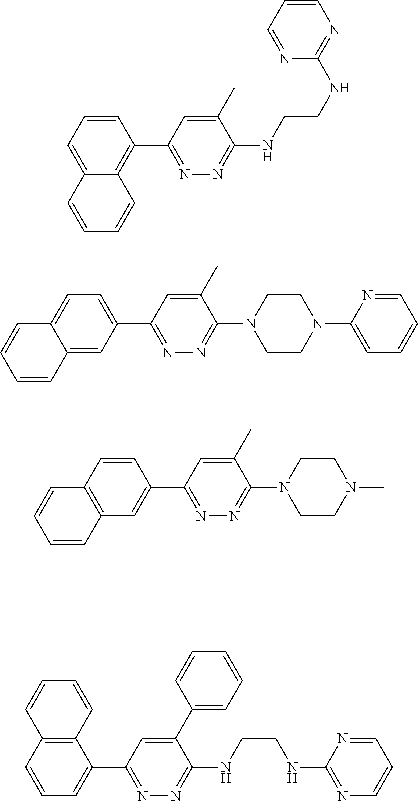 [SMILES: Cc1cc(-c2cccc3ccccc23)nnc1NCCNc1ncccn1 ; Cc1cc(-c2ccc3ccccc3c2)nnc1N1CCN(c2ccccn2)CC1 ; Cc1cc(-c2ccc3ccccc3c2)nnc1N1CCN(C)CC1 ; c1ccc(-c2cc(-c3cccc4ccccc34)nnc2NCCNc2ncccn2)cc1] 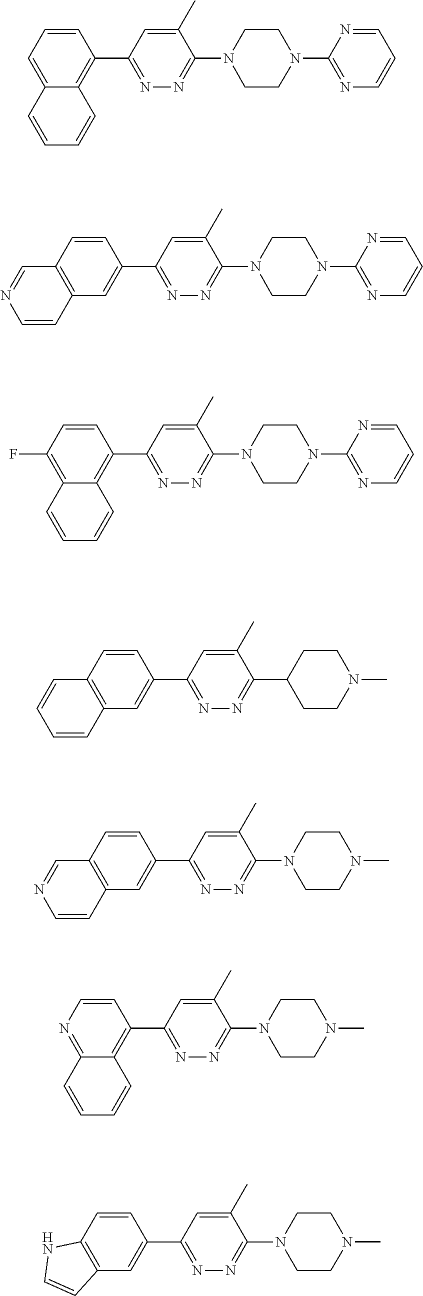 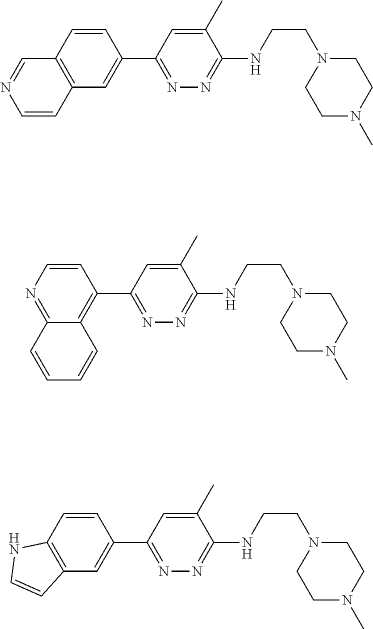 [SMILES: Cc1cc(-c2ccc3cnccc3c2)nnc1NCCN1CCN(C)CC1 ; Cc1cc(-c2ccnc3ccccc23)nnc1NCCN1CCN(C)CC1 ; Cc1cc(-c2ccc3[nH]ccc3c2)nnc1NCCN1CCN(C)CC1]
or a pharmaceutically acceptable salt thereof.

8. The compound of claim 1, having the structure: 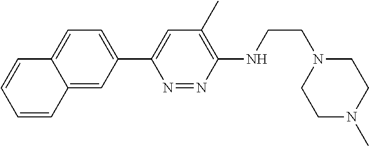 [SMILES: Cc1cc(-c2ccc3ccccc3c2)nnc1NCCN1CCN(C)CC1]
or a pharmaceutically acceptable salt thereof.

9. The compound of claim 1, having the structure: 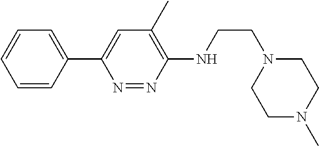 [SMILES: Cc1cc(-c2ccccc2)nnc1NCCN1CCN(C)CC1]
or a pharmaceutically acceptable salt thereof.

10. The compound of claim 1, wherein the compound is a compound of Formula (I), wherein R₂ is phenyl or naphthyl; or a pharmaceutically acceptable salt thereof.

11. The compound of claim 10, wherein R₃, R₄ together with the N to which they are attached form 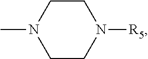
where R₅ is methyl;
or a pharmaceutically acceptable salt thereof.

12. A pharmaceutical composition comprising a compound of claim 1 and a pharmaceutically acceptable excipient.

13. The pharmaceutical composition of claim 12, wherein the compound has the structure: 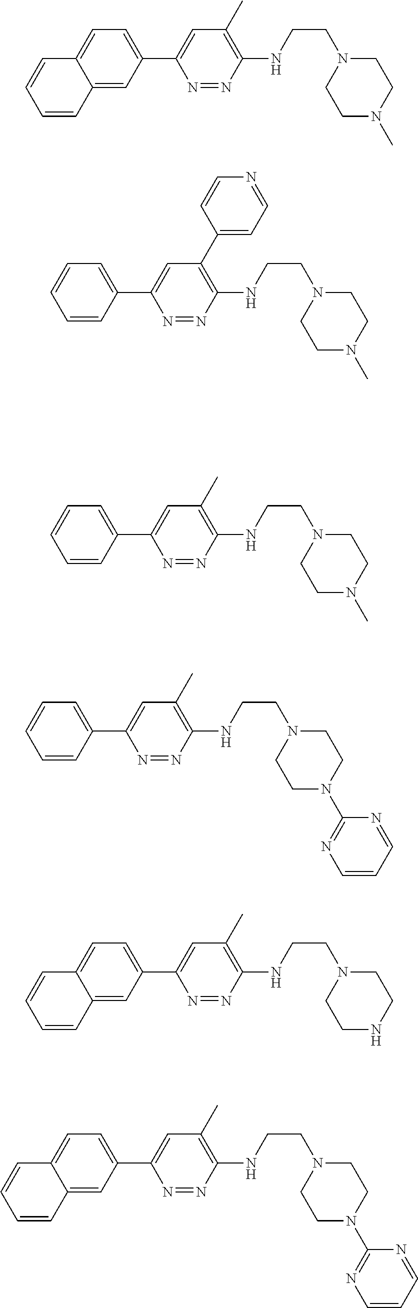 [SMILES: Cc1cc(-c2ccc3ccccc3c2)nnc1NCCN1CCN(C)CC1 ; CN1CCN(CCNc2nnc(-c3ccccc3)cc2-c2ccncc2)CC1 ; Cc1cc(-c2ccccc2)nnc1NCCN1CCN(C)CC1 ; Cc1cc(-c2ccccc2)nnc1NCCN1CCN(c2ncccn2)CC1 ; Cc1cc(-c2ccc3ccccc3c2)nnc1NCCN1CCNCC1 ; Cc1cc(-c2ccc3ccccc3c2)nnc1NCCN1CCN(c2ncccn2)CC1] 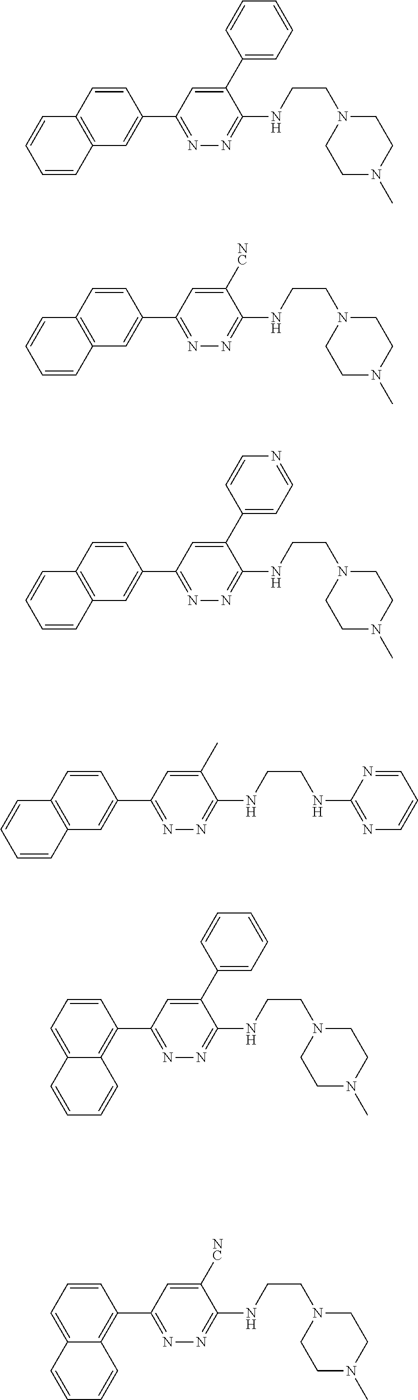 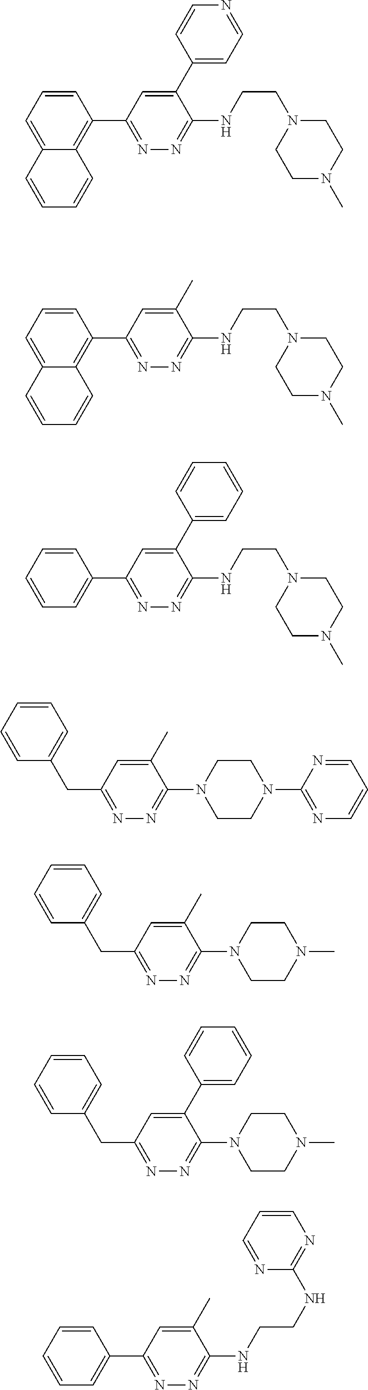 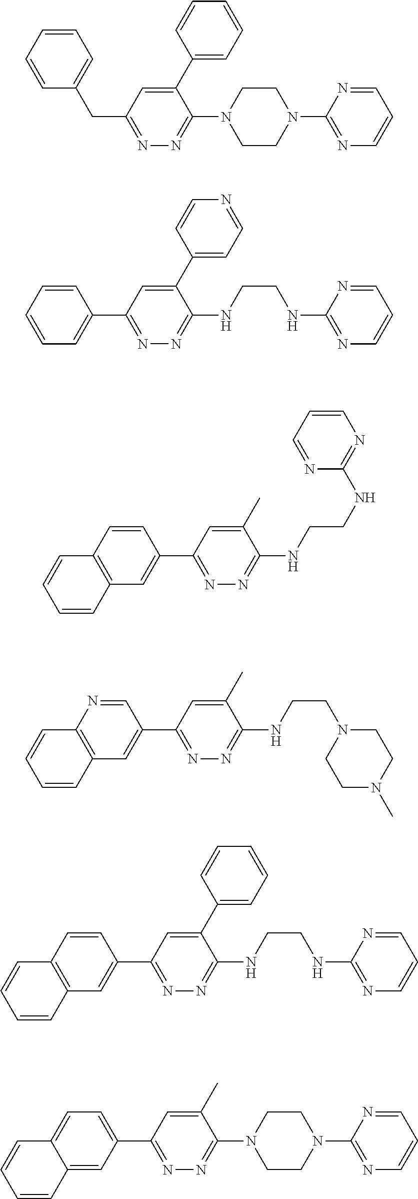 [SMILES: c1ccc(Cc2cc(-c3ccccc3)c(N3CCN(c4ncccn4)CC3)nn2)cc1 ; c1ccc(-c2cc(-c3ccncc3)c(NCCNc3ncccn3)nn2)cc1 ; Cc1cc(-c2ccc3ccccc3c2)nnc1NCCNc1ncccn1 ; Cc1cc(-c2cnc3ccccc3c2)nnc1NCCN1CCN(C)CC1 ; c1ccc(-c2cc(-c3ccc4ccccc4c3)nnc2NCCNc2ncccn2)cc1 ; Cc1cc(-c2ccc3ccccc3c2)nnc1N1CCN(c2ncccn2)CC1] 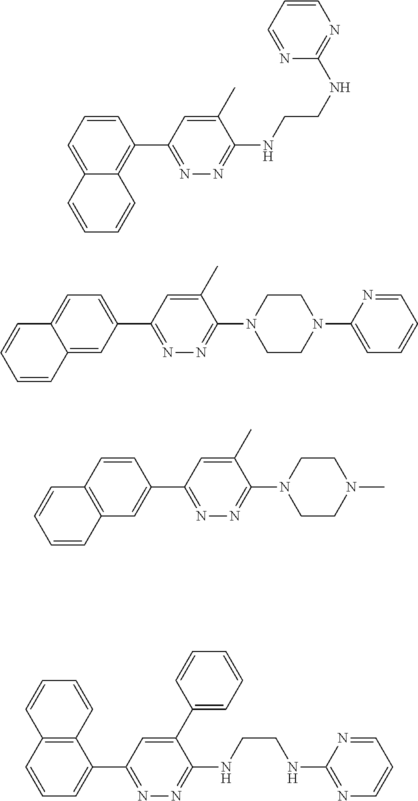 [SMILES: Cc1cc(-c2cccc3ccccc23)nnc1NCCNc1ncccn1 ; Cc1cc(-c2ccc3ccccc3c2)nnc1N1CCN(c2ccccn2)CC1 ; Cc1cc(-c2ccc3ccccc3c2)nnc1N1CCN(C)CC1 ; c1ccc(-c2cc(-c3cccc4ccccc34)nnc2NCCNc2ncccn2)cc1] 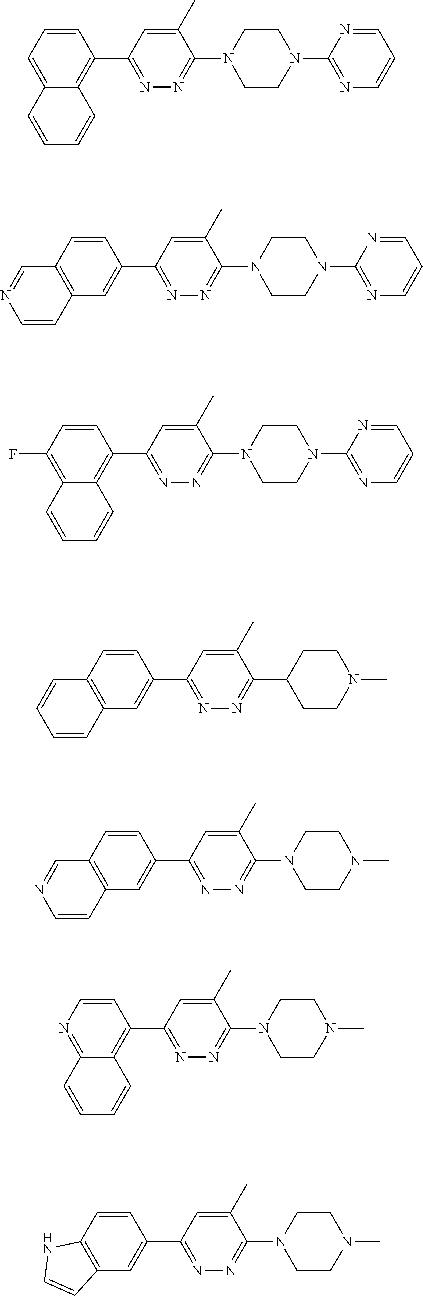 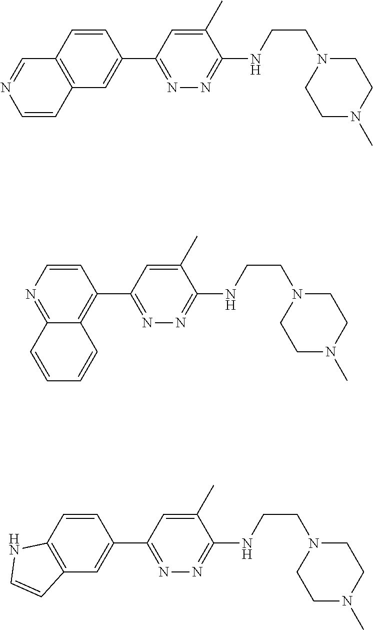 [SMILES: Cc1cc(-c2ccc3cnccc3c2)nnc1NCCN1CCN(C)CC1 ; Cc1cc(-c2ccnc3ccccc23)nnc1NCCN1CCN(C)CC1 ; Cc1cc(-c2ccc3[nH]ccc3c2)nnc1NCCN1CCN(C)CC1]
or a pharmaceutically acceptable salt thereof.

14. The pharmaceutical composition of claim 12, wherein the compound is a compound having the structure:

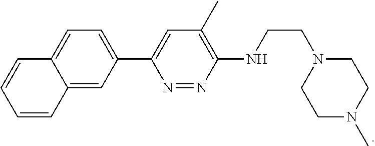 [SMILES: Cc1cc(-c2ccc3ccccc3c2)nnc1NCCN1CCN(C)CC1]

15. The pharmaceutical composition of claim 12, wherein the compound is a compound having the structure:

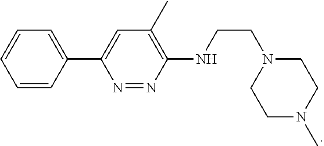 [SMILES: Cc1cc(-c2ccccc2)nnc1NCCN1CCN(C)CC1]

16. A method of treating a neuropsychiatric, cognitive or behavioral disorder, the method comprising administering to a subject in need thereof a therapeutically effective amount of a compound of claim 1.

17. The method of claim 16, wherein the neuropsychiatric, cognitive or behavioral disorder is associated with a neurodegenerative disease.

18. The method of claim 17, wherein the neurodegenerative disease is Alzheimer's disease, another tauopathy, or dementia.

19. The method of claim 16, wherein the neuropsychiatric, cognitive or behavioral disorder comprises aggressive behavior.

20. A method of treating a neurodegenerative disease in a subject in need thereof, comprising administering to the subject a therapeutically effective amount of a compound of claim 1.
